SEQUENCE LISTING (74 sequences)

SEQ ID NO: 1 - DNA, 2315 bp, Epichloe festucae
cttataactatattaggggagctaaagatatatattactaaatgtaataaattataataaaagcatataatctctaatataatagctttctctataaaagagaagcttttatccttctatttatataatagaagatagctttaattcaatccttttttctacctttatcatcctgcgaggggatatcttttccaagtcacgtgcctagcgcacgataatacctatgccctcgttgtgccacatgtgtggatagcctagatagctgaatacatcatcatcactttccacaccgtctcacatcttccactgccgattccacagaccccgagtcaccgacagagggaacgtctcgaggcggatcaggccaacgtcggcgatatccccaaagaaccacggcgccgtccatgcaaaccagcaaagcacccaagcccggcccaggaaacgggtcaggctggacgactcccgcatccccgtcttcttaccagcccactgcacaacatcctcggccacgatgccgttggcctgcatgagaaagaagatccaagtcccggcgcaggagcgtcggtgcggatcggccatccgcgcagcggcaacgtgcatcaaggccgatagcagaaaagaagtgtagacctggacgtatctcgagacaagggagcctttgagggcgcgcatgacttcccaggcaatccattgaccgatcgactgccagggctgggacagacgttagcactcctcaccggccggccgctgcaggcatatgtacgtaccttgcggaacgtctggtgccagacgcggccccagaagcgtcgcagcgtccatgcgtcccgccagcggccgactgcaggccggttgtggtgcggcctggtccagaagcacccgctggccatgcccatggcgcatacagcacggtatggcatgtcgatgttcaacaaggccccgattccaagtgaatagacggtcagaaaacgggcgccgccctttgtatcgctggctccgacgccctcgtgagcaaacaagtccgcagatagcagcgagcgtccgagatcctggacaaggtagagccagccgagattaagagcgctcgcccgcaggtagtcccgaatcgtcgtgttgctgggcatggcctcgggaaggtgagggatggcccagctaaagccaatccctcgagcagagaagcagtgcgcaaggcaccactcccatttcccccaactcgataggctcctgtatctgacgggcgtcttttccttgtcatccaactcgtaatagtcggcgtagatgtctctctcgatgacgagcatctggaatgccttgcaaaggcacgcgcaaccgactattttactcgacgcgtcaacactcttcagcacggtaggggcttggatatgtatgtactcatcccgaaggcataatcactccctacctcgtcctgtggtgcgcagcgcacgacactaagaaaggcgcataatattatgccgcaatagacaaggcgaatgggccgccgagtgtagtatccgaggtaaagagaaatagtcatcaagatggggaacattgggagcgcaagaacccgggttgatgccatgattatcccggcagcaagctccaaggatcgtcacggggtaccaactgttgccgctgagtaggcaaaccggaaatctgtgttccgccagagcacagtcacgaaaggtcgggcttcgggattaatgcaagaggatatgaagcagaaccggatcggccttgatggggcttatagcatgggtgcggtccagccgaggtccgtagcgagtctgcagagcagcaacaccaggggcacatcgaggatattggcggtacgtaataacgaagcaacatatccaactatgcccatctgtttctaggctgcagtcagtggtgcggcttgatgtatacggtccatgtacgccttcggccaatgtccgacattccctcggcacgcggcttgatgctcggatgcgagaacagacggaagacggaccaagagacgcggcagcagcagacctaggcactccattaataatagactaaaatatttgtcttgattgcatagaacgtcaagcgcggcattgcggggcctcgtgcagagagatctgcaacgaggcgtctttgatgcctgcagcgatcaatggcggtagcacacgaccaccttgttcaggcaaagacttgccgaatgggccgttttctgcctttcagcctccttctcccctgccagccctgtccatgccctgccgttgtcgtgactgggagctcgctctcccttttctccaagcttcgactgttcccagcaaacccccatagagacacac

SEQ ID NO: 2 - DNA, 1004 bp, Epichloe festucae
atgctcgtcatcgagagagacatctacgccgactattacgagttggatgacaaggaaaagacgcccgtcagatacaggagcctatcgagttgggggaaatgggagtggtgccttgcgcactgcttctctgctcgagggattggctttagctgggccatccctcaccttcccgaggccatgcccagcaacacgacgattcgggactacctgcgggcgagcgctcttaatctcggctggctctaccttgtccaggatctcggacgctcgctgctatctgcggacttgtttgctcacgagggcgtcggagccagcgatacaaagggcggcgcccgttttctgaccgtctattcacttggaatcggggccttgttgaacatcgacatgccataccgtgctgtatgcgccatgggcatggccagcgggtgcttctggaccaggccgcaccacaaccggcctgcagtcggccgctggcgggacgcatggacgctgcgacgcttctggggccgcgtctggcaccagacgttccgcaaggtacgtacatatgcctgcagcggccggccggtgaggagtgctaacgtctgtcccagccctggcagtcgatcggtcaatggattgcctgggaagtcatgcgcgccctcaaaggctcccttgtctcgagatacgtccaggtctacacttcttttctgctatcggccttgatgcacgttgccgctgcgcggatggccgatccgcaccgacgctcctgcgccgggacttggatcttctttctcatgcaggccaacggcatcgtggccgaggatgttgtgcagtgggctggtaagaagacggggatgcgggagtcgtccagcctgacccgtttcctgggccgggcttgggtgctttgctggtttgcatggacggcgccgtggttctttggggatatcgccgacgttggcctgatccgcctcgagacgttccctctgtcggtgactcggggtctgtggaatcggcagtggaagatgtga

SEQ ID NO: 3 - protein, 315 aa, Epichloe festucae
  DOMAIN (150)..(239): MBOAT 2 (Membrane bound O-acyl transferase) domain
MLVIERDIYADYYELDDKEKTPVRYRSLSSWGKWEWCLAHCFSARGIGFSWAIPHLPEAMPSNTTIRDYLRASALNLGWLYLVQDLGRSLLSADLFAHEGVGASDTKGGARFLTVYSLGIGALLNIDMPYRAVCAMGMASGCFWTRPHHNRPAVGRWRDAWTLRRFWGRVWHQTFRKPWQSIGQWIAWEVMRALKGSLVSRYVQVYTSFLLSALMHVAAARMADPHRRSCAGTWIFFLMQANGIVAEDVVQWAGKKTGMRESSSLTRFLGRAWVLCWFAWTAPWFFGDIADVGLIRLETFPLSVTRGLWNRQWKM

SEQ ID NO: 4 - DNA, 2791 bp, Epichloe festucae
aggaagtcacgcaggagtgggattagtaccttgaaaagcccctggaaatacctgcataggacagatgccattaactctgctacgtaacaaaaaataacaaaaataaagggaagcaattaaggaatcaacaccccatctaaccttaccgcttggtccaccgcctactcagcctccaaatgcaactcaggcttataatagacggtaacataggggttagtccccttgaatgaaaaagaaatgtcgtggtgcacagccgtgagctggttgtccgagttcatactaccttactgttagccaagggcaaagggaaagaaggccaggggcaacaagaacagacaagcacttgaggtagtttttcttatagccagggtaaataccatcccatccctggctcaggaaaaagcggtccattccatcagcaatctccaagtcgggtcggccatagtaatgggctgggaggtaaatcttaggagccgggaactcggcgccaggccggacctcaaagttgaagatgactgccgcacagccattggggttgatgggtagatccttatcgtctggccacccctcgggaacattgagcacggccttccagagtgtcctcaaggcctgcgtgcccttttcaatacaaggccccattagcaaaccacctagggtatagatgtcaaccatgacgttaaaggcagttcggtgggtaacaaaatagatcttgatacgtgagcttgtatagtcgtctttgagcaccgagtcgatggcgaaaaagatcggatgtgcccctgcagctgcggcacttttggacgttgagtattgctggagcatagcgagcactggcttatacggaataacgtcgtcccctaaccccaagattgcatcccagataacttcctccttatcaaggccagtggcgatagacttccagtgggaggcaaaggctagcttacactggatgtgattgtcgggaaagtcgtggctgagcatgcactgaactccctgccggatctggaactcgtccaccttggccttgagggtggccgccgtatctcgatccaggaagagcttagatacaaagtggttaaaccagcgcatgtcctgggacggcaggacctgttgctggcggaccatggcatctatcgcagccttctggttaatggggtcttctgctgtgccggagatattgctgataggtatatgagctgtccgaagcgtcatcttcccatcgtggtagttgagactgagctggataggggtgcaatcgtagttgagaaacaccttccagaacagatcaccgccatccccaggccttgggccacagctagggacaatatacgcgtgtatgaatgccaggactctgtactgctccggcgtcgagtactgcccccacttcatcatcttgccagccaggggggcaagtttggaccaccagtacctctcgttctcattggcgaaccctagaccctgagctagggcctgccatggctcaggcggcgtcacatctgctggtttcgatggcgtctcgcctacgcgagtggaacaggtgcccatcttggtcggtcgtgcaaatgggtttcgaatcagagactggccgtcagatgaggtctctaagccagtaaagcgagccacgtcgggttgagcaagaatcagggctacttggacgttgcgtcctggcacgattggactcttatagacaacggctcgttatgtcggtggcggctgcacgacaagtctgatatcgctctgcccatctacgcataaagatgtctgcaaacaaggcataattctagtcaaagtgggctgccttcgggcctgttcaagtgtcgtagatgtgtggcatacaagcaacttcccgagggccttgttactcgactgggaacagctgcattgcatattataggcaatatattggctcgcgatgctgcacatgctgcactatgcagcatctaatttccataatgcagtgtttgccgagagagtgttttgtgctgacaccgtttgctgcagcacccagggtctacatgtacgcatgctgctctccttgccatcatcatactaccctggcctgcaagagaggagcagggtgccatagtcgggtgccgcgtggagatttctttggcttttcgtatcccatcaatatggcaagtctgacctgatgctgctattctctaaccgtcttctgttggatatagcagcctcacatcttaggcatcactcatgataatacctccctattcgaggccatgggttatcatagattgtggtaccttggcacttagggcaaagaccctctttacccttatacctatgcccttgttataccacatatataaatagcctagatagctaaatacattattattactttttacactatcttataggttataagcctagcgcttcctatcacgcctagttacacttagctatttttttactattaaagagttatatataattacactagaaatcttaataatatccagaagcgtataattacgtgatttaaggggtagtagataatcccctcctttaatagataactaagttataaaggcataattaaattaattaagtaagcctttatttaaaaaaaaaacatctcttattatattaataggatttaaatacataacctttatagcgttaattaacgaattataattatagtaattataatactattagtgtaaataaccacatgttaagatttaggaataactttaaaaaagttagacccttctaggtaaatcttagtggatagcaggctaggacaagacattccttgcaaaactgcctt

SEQ ID NO: 5 - DNA, 1495 bp, Epichloe festucae
cgcaacgtccaagtagccctgattcttgctcaacccgacgtggctcgctttactggcttagagacctcatctgacggccagtctctgattcgaaacccatttgcacgaccgaccaagatgggcacctgttccactcgcgtaggcgagacgccatcgaaaccagcagatgtgacgccgcctgagccatggcaggccctagctcagggtctagggttcgccaatgagaacgagaggtactggtggtccaaacttgcccccctggctggcaagatgatgaagtgggggcagtactcgacgccggagcagtacagagtcctggcattcatacacgcgtatattgtccctagctgtggcccaaggcctggggatggcggtgatctgttctggaaggtgtttctcaactacgattgcacccctatccagctcagtctcaactaccacgatgggaagatgacgcttcggacagctcatatacctatcagcaatatctccggcacagcagaagaccccattaaccagaaggctgcgatagatgccatggtccgccagcaacaggtcctgccgtcccaggacatgcgctggtttaaccactttgtatctaagctcttcctggatcgagatacggcggccaccctcaaggccaaggtggacgagttccagatccggcagggagttcagtgcatgctcagccacgactttcccgacaatcacatccagtgtaagctagcctttgcctcccactggaagtctatcgccactggccttgataaggaggaagttatctgggatgcaatcttggggttaggggacgacgttattccgtataagccagtgctcgctatgctccagcaatactcaacgtccaaaagtgccgcagctgcaggggcacatccgatctttttcgccatcgactcggtgctcaaagacgactatacaagctcacgtatcaagatctattttgttacccaccgaactgcctttaacgtcatggttgacatctataccctaggtggtttgctaatggggccttgtattgaaaagggcacgcaggccttgaggacactctggaaggccgtgctcaatgttcccgaggggtggccagacgataaggatctacccatcaaccccaatggctgtgcggcagtcatcttcaactttgaggtccggcctggcgccgagttcccggctcctaagatttacctcccagcccattactatggccgacccgacttggagattgctgatggaatggaccgctttttcctgagccagggatgggatggtatttaccctggctataagaaaaactacctcaagtgcttgtctgttcttgttgcccctggccttctttccctttgcccttggctaacagtaaggtagtatgaactcggacaaccagctcacggctgtgcaccacgacatttctttttcattcaaggggactaacccctatgttaccgtctattataagcctgagttgcatttggaggctgag

SEQ ID NO: 6 - protein, 479 aa, Epichloe festucae
  DOMAIN (76)..(436): Aromatic prenyl transferase, DMATS type domain
  VARIANT (235)..(235): Aspartic acid to glutamic acid substitution in E. festucae strains AR127, AR128, and AR166
RNVQVALILAQPDVARFTGLETSSDGQSLIRNPFARPTKMGTCSTRVGETPSKPADVTPPEPWQALAQGLGFANENERYWWSKLAPLAGKMMKWGQYSTPEQYRVLAFIHAYIVPSCGPRPGDGGDLFWKVFLNYDCTPIQLSLNYHDGKMTLRTAHIPISNISGTAEDPINQKAAIDAMVRQQQVLPSQDMRWFNHFVSKLFLDRDTAATLKAKVDEFQIRQGVQCMLSHDFPDNHIQCKLAFASHWKSIATGLDKEEVIWDAILGLGDDVIPYKPVLAMLQQYSTSKSAAAAGAHPIFFAIDSVLKDDYTSSRIKIYFVTHRTAFNVMVDIYTLGGLLMGPCIEKGTQALRTLWKAVLNVPEGWPDDKDLPINPNGCAAVIFNFEVRPGAEFPAPKIYLPAHYYGRPDLEIADGMDRFFLSQGWDGIYPGYKKNYLKCFMNSDNQLTAVHHDISFSFKGTNPYVTVYYKPELHLEAE

SEQ ID NO: 7 - DNA, 3134 bp, Epichloe festucae
ggcgcgcacttaataggcttactaatattaactctaatgccttaataccagagattttagatattatagaggctaataatataggaatagttatataataatacactctactattataagatatattatatattaataattagtctacataagaattttaggcgtaaatattattataattataagagaaggttatagcaaaggtagcgctacaggtagagataatttagctagggtaaggggtggttagggaatggttagacacttagtatagagctactatataatttactataggtagctagtattagttattgcagctaccttagctacctttaggggtgctaattacgcgtacgcgtttgcctgataagcacgccctttaaataaatcgctgcctacgagtcactgaggagcatcccccgaattcttccactaggatcccacttctttccaacacggagcactcgctctttggcctcctctgaccagtaattagacggtctttccaacacatgcgcatcccccacaaacgtgcccaagacaaatggctccagcttacgcatataatccacgatccaggacgtctgctcatcgtcttgggcggaatcgtcgtagattcctagtagggatacgtaatggtcgctatgtatgctcatggccatgtcgggaagggtgcgggcagtggcggtcttcatcggctgccaatatgccaatgcgcgcggatgctttagtcctgtaaacatgtccttacaagccaccactaggtcggtgccaggcttgagaaagacgctatcggtgaagtaacgagcaccttgcggcaggactgtcttatctagaacgtagtcgctagcgaagtcagtcgccacaaagtcctgggcgtgaatcgctcctgggggtgggtgtcgatgaagggtgtgagcattcgccgggtatcctcatccgagtctccgaatgctgttgcgagcacgtgtagccccggctggttgagttgggggactgtaaatccgaacacgccaacctcgacgttctcgtccagcgtagttaagagcttgaaacctcgtgagcttggctggggacatggacacgagctactcacgcggtcgagccaagggaaaacctggtcatacatggtcgccggccagatgtacgtgctccggttgcaaaccggcatcggtcgagtctcgaggtagaagcgtgtgacaatggcagggaactctgtttagttagatttgccacggctttaatgcggaaggctttcagagagggactcacccggcccagctccgcgagcggcccaatacaaatcagcgttttcttctttgtcacagtgcttaatgcagccatcggcggtaacaatgtcgattccagagacagactcgcatgcgtacccatagccctagacgttgccgtcagtggatgaagagaaacggataggcaagcatggggcaggcagagcaagcacgcatacccggcagttgagtccgattccagcttggaggaggtagccacctaggccaatgccccctgagtggcctacgggaaagaatcgctgcttctgggcaagaaactcgagcaattctcccgttagggtactgggagaggcctggacttggtgtgtttcttcatcgtagcttagatagctaaagtccttgagatcaatgaggatggcatcatggcgcatggtccagcaggagagactgtggccacctgagcggacggccacaggggcggcggactccacggccagcctcacagccgctacgatgtggtcgagcgtcgtggcctggacgattgcgcgtgggaaatgctcgggccgcctgccgttgaagactctgcaccgtgcttcatcgtactggttgaggggggagtcggtccgccacagaatggggagcttctcggttgaggagtctcgcatgttgcgggagagacagtctcgcgtgttgccgggaagagggtctcccatgctgtcaagaagagaagcgattcaaagcggtagtgcttgatggttccggggatgatacacgcttacttcccttgctattatagccaacagatcccatttcctcgggcatttcttcaatccgggacagcacaggcggggcgcgtatcggccgctctcctcggcatcatatggcattgaatgcaaccatctcccgtaatgcatatgcagcctgcaatatggagcaataattaaacgcaagataccgcaccgtgcgctttgttcatgtacatgaaatattactctcgcggtatgcgtgtcagaacaagggtgagtctcaccgttggcggagccaacatggtcaggaatcaggatgaggctgcgatagagcccgaggaggggtcgcaatggccttgttgcatgtgtagccggccacgtctgcaggtgtagtatgcagtgtattcgccaaagttccaatacgttgatggctaggaggggggttactggtaataatgaggatggatgcgtcatcttgatgcgccgagcgccaaaccagctaccctggaccgaaccaaggggtcatagggtagcctgtgctactacagttggcgctgtcaggcagcgcactctagatagtccggccccaggccctgtggtgcaagaagtcaggaagaagggaaaaacaaggttgattcacaactagctaggcggtgcggcgggctcgtaagagctggctggccgcacacgggctaactacttatctaccggtcaggttaccctgtatagcgccaaggtggtatagggtagctgatgctggccctagggcacggagttgctgacttccaatctccagcctgtttctcctgcctcagtaaagccgacaactttgcgcagcgactctagtcccatcgactgtaagttttcaagaggcagaagaaacgactagctaaacttaccaaatcttgtgtggcaaatatggagatatagagcaacttccttgctgttggaaggggtctgaatcttataagtccctgcacataggaatgggccaccccccttatgggccactcaaaaaatcatggtccattttgacaaccatcaattcaatcgctatttattcaccactagttcgtactacgatgcagccatcatcaatttcggatactgcagactcctttccgtcagattctcc

SEQ ID NO: 8 - DNA, 1520 bp, Epichloe festucae
atgcgagactcctcaaccgagaagctccccattctgtggcggaccgactcccccctcaaccagtacgatgaagcacggtgcagagtcttcaacggcaggcggcccgagcatttcccacgcgcaatcgtccaggccacgacgctcgaccacatcgtagcggctgtgaggctggccgtggagtccgccgcccctgtggccgtccgctcaggtggccacagtctctcctgctggaccatgcgccatgatgccatcctcattgatctcaaggactttagctatctaagctacgatgaagaaacacaccaagtccaggcctctcccagtaccctaacgggagaattgctcgagtttcttgcccagaagcagcgattctttcccgtaggccactcagggggcattggcctaggtggctacctcctccaagctggaatcggactcaactgccgggtatgcgtgcttgctctgcctgccccatgcttgcctatccgtttctcttcatccactgacggcaacgtctagggctatgggtacgcatgcgagtctgtctctggaatcgacattgttaccgccgatggctgcattaagcactgtgacaaagaagaaaacgctgatttgtattgggccgctcgcggagctgggccgggtgagtccctctctgaaagccttccgcattaaagccgtggcaaatctaactaaacagagttccctgccattgtcacacgcttctacctcgagactcgaccgatgccggtttgcaaccggagcacgtacatctggccggcgaccatgtatgaccaggttttcccttggctcgaccgcgtgagtagctcgtgtccatgtccccagccaagctcacgaggtttcaagctcttaactacgctggacgagaacgtcgaggttggcgtgttcggatttacagtcccccaactcaaccagccggggctacacgtgctcgcaacagcattcggagactcggatgaggatacccggcgaatgctcacacccttcatcgacacccacccccaggagcgattcacgcccaggactttgtggcgactgacttcgctagcgactacgttctagataagacagtcctgccgcaaggtgctcgttacttcaccgatagcgtctttctcaagcctggcaccgacctagtggtggcttgtaaggacatgtttacaggactaaagcatccgcgcgcattggcatattggcagccgatgaagaccgccactgcccgcacccttcccgacatggccatgagcatacatagcgaccattacgtatccctactaggaatctacgacgattccgcccaagacgatgagcagacgtcctggatcgtggattatatgcgtaagctggagccatttgtcttgggcacgtttgtgggggatgcgcatgtgttggaaagaccgtctaattactggtcagaggaggccaaagagcgagtgctccgtgttggaaagaagtgggatcctagtggaagaattcgggggatgctcctcagtgactcgtag

SEQ ID NO: 9 - protein, 388 aa, Epichloe festucae
  DOMAIN (39)..(174): FAD binding domain
MRDSSTEKLPILWRTDSPLNQYDEARCRVFNGRRPEHFPRAIVQATTLDHIVAAVRLAVESAAPVAVRSGGHSLSCWTMRHDAILIDLKDFSYLSYDEETHQVQASPSTLTGELLEFLAQKQRFFPVGHSGGIGLGGYLLQAGIGLNCRGYGYACESVSGIDIVTADGCIKHCDKEENADLYWAARGAGPEFPAIVTRFYLETRPMPVCNRSTYIWPATMYDQVFPWLDRDFVATDFASDYVLDKTVLPQGARYFTDSVFLKPGTDLVVACKDMFTGLKHPRALAYWQPMKTATARTLPDMAMSIHSDHYVSLLGIYDDSAQDDEQTSWIVDYMRKLEPFVLGTFVGDAHVLERPSNYWSEEAKERVLRVGKKWDPSGRIRGMLLSDS

SEQ ID NO: 10 - DNA, 22 bp, Artificial
  synthetic oligonucleotide synthesised in laboratory
cttataactatattaggggagc

SEQ ID NO: 11 - DNA, 21 bp, Artificial
  synthetic oligonucleotide synthesised in laboratory
gtgtgtctctatgggggtttg

SEQ ID NO: 12 - DNA, 21 bp, Artificial
  synthetic oligonucleotide synthesised in laboratory
aggaagtcacgcaggagtggg

SEQ ID NO: 13 - DNA, 25 bp, Artificial
  synthetic oligonucleotide synthesised in laboratory
aaggcagttttgcaaggaatgtctt

SEQ ID NO: 14 - DNA, 22 bp, Artificial
  synthetic oligonucleotide synthesised in laboratory
ggcgcgcacttaataggcttac

SEQ ID NO: 15 - DNA, 21 bp, Artificial
  synthetic oligonucleotide synthesised in laboratory
ggagaatctgacggaaaggag

SEQ ID NO: 16 - DNA, 1496 bp, Epichloe festucae
atgattgcgaaaaatattgaactcaatggcttggatccggcaaccagggcattggacattctatactggaaaaatcactgcatcaaacagctagaatctctcctatgcgccacagattcatactgcactgcagacaaggccgctcaactacgcattttgtcagagttggtgctccccaatcttggccctcggccgtccaatgccactgggccatcctatcttacacgaagtggttccccaataatgttaagtctaaatacaacatcatcaaaaaactgcgtcagatattgctgggagattctaggggcgactggcgcaagtaatgatgatcctttggcagtccaagttgctaaggatgtagtggcttctctgtctgctacttttcgcctttcaacaaaatggagcgaaactctactgtccaattttgcagtaacaccagaccaagctcgacaagttattaacatgctacccgagtggattcaaggcttcgtacctgagggaatggagtgcgattttccaaagagaatcccgttcgccatgacatcattcgacctaaatggctccaatgtagctatgaagctctacgttaatccaagggtaaaggagattttaactggtactccctcatcagacttggtctgggagttcctccgaaatttaacaccagaaatgaaaccacgagcggtcgacttgcttgagaggtaagaatggctttgaactttcgcccaccttgtcagccccatacgctaagcgctaactccccacacattaacaggtttattaccgataattcaggcccgtctgctattgagcttgtaggtattgactgcgttgacgacgctcacctatcaaatgcaagggtcaagctttacgttcataccatgagcagctcatttaacaccgtaaagaattatgttactcttgggggtgcaatctgggatgaacaaacccaaaagggcttaggaatactacaaagtatttggcacctattgcttcaggagccagagggtatttctgacaatggattcgacaagcctgtgaacgactcttccatgttatgccaaaagctatattttagtttcgagctacgcccaggtacagacttccctcaggtgaagacctatgtgccaacttggaactatcttcgaaccgacggggaaactatccagaactatgaggcgatcttccgagcttgtgaccatccttggggtgaagataggacgtacggcaaaatttttcaagatgcattgtaagttatcccttcagattagcgctaaaaggagtttgagatactcctcaatgcaagctattaggttgtgaaattgccactactaattggagctttttatagcggacctgcaaccgagagtcggaaaaaacccattcactgcgacgcatcttttctgtttaccgaagaaactggtgtctaccagacgctgtatttcagtcctccgattgagggggaaacagaagtccagtcaaatctcgttgcttga

SEQ ID NO: 17 - protein, 439 aa, Epichloe festucae
  DOMAIN (19)..(386): Aromatic prenyl transferase, DMATS type domain
MIAKNIELNGLDPATRALDILYWKNHCIKQLESLLCATDSYCTADKAAQLRILSELVLPNLGPRPSNATGPSYLTRSGSPIMLSLNTTSSKNCVRYCWEILGATGASNDDPLAVQVAKDVVASLSATFRLSTKWSETLLSNFAVTPDQARQVINMLPEWIQGFVPEGMECDFPKRIPFAMTSFDLNGSNVAMKLYVNPRVKEILTGTPSSDLVWEFLRNLTPEMKPRAVDLLERFITDNSGPSAIELVGIDCVDDAHLSNARVKLYVHTMSSSFNTVKNYVTLGGAIWDEQTQKGLGILQSIWHLLLQEPEGISDNGFDKPVNDSSMLCQKLYFSFELRPGTDFPQVKTYVPTWNYLRTDGETIQNYEAIFRACDHPWGEDRTYGKIFQDAFGPATESRKKPIHCDASFLFTEETGVYQTLYFSPPIEGETEVQSNLVA

SEQ ID NO: 18 - DNA, 1496 bp, Epichloe festucae
atgattgcgaaaaatattgaactcaatggcttggatccggcaaccagggcattggacattctatactggaaaaatcactgcatcaaacagctagaatctctcctatgcgccacagattcatactgcactgcagacaaggccgctcaactacgcattttgtcagagttggtgctccccaatcttggccctcggccgtccaatgccactgggccatcctatcttacacgaagtggttccccaataatgttaagtctaaatacaacatcatcaaaaaactgcgtcagatattgctgggagattctaggggcgactggcgcaagtaatgatgatcctttggcagtccaagttgctaaggatgtagtggcttctctgtctgctacttttcgcctttcaacaaaatggagcgaaactctactgtccaattttgcagtaacaccagaccaagctcgacaagttattaacatgctacccgagtggattcaaggcttcgtacctgagggaatggagtgcgattttccaaagagaatcccgttcgccatgacatcattcgacctaaagggctccaatgtagctatgaagctctacgttaatccaagggtaaaggagattttaactggtactccctcatcagacttggtctgggagttcctccgaaatttaacaccagaaatgaaaccacgagcggtcgacttgcttgagaggtaagaatggctttgaactttcgcccaccttgtcagccccatacgctaagcgctaactccccacacattaacaggtttattaccgataattcaggcccgtctgctattgagcttgtaggtattgactgcgttgacgacgctcacctatcaaatgcaagggtcaagctttacgttcataccatgagcagctcatttaacaccgtaaagaattatgttactcttgggggtgcaatctgggatgaacaaacccaaaagggcttaggaatactacaaagtatttggcacctattgcttcaggagccagagggtatttctgacaatggattcgacaagcctgtgaacgactcttccatgttatgccaaaagctatattttagtttcgagctacgcccaggtacagacttccctcaggtgaagacctatgtgccaacttggaactatcttcgaaccgacggggaaactatccagaactatgaggcgatcttccgagcttgtgaccatccttggggtgaagataggacgtacggcaaaatttttcaagatgcattgtaagttatcccttcagattagcgctaaaaggagtttgagatactcctcaatgcaagctattaggttgtgaaattgccactactaattggagctttttatagcggacctgcaaccgagagtcggaaaaaacccattcactgcgacgcatcttttctgtttaccgaagaaactggtgtctaccagacgctgtatttcagtcctccgattgagggggaaacagaagtccagtcaaatctcgttgcttga

SEQ ID NO: 19 - protein, 439 aa, Epichloe festucae
  DOMAIN (19)..(386): Aromatic prenyl transferase, DMATS type domain
MIAKNIELNGLDPATRALDILYWKNHCIKQLESLLCATDSYCTADKAAQLRILSELVLPNLGPRPSNATGPSYLTRSGSPIMLSLNTTSSKNCVRYCWEILGATGASNDDPLAVQVAKDVVASLSATFRLSTKWSETLLSNFAVTPDQARQVINMLPEWIQGFVPEGMECDFPKRIPFAMTSFDLKGSNVAMKLYVNPRVKEILTGTPSSDLVWEFLRNLTPEMKPRAVDLLERFITDNSGPSAIELVGIDCVDDAHLSNARVKLYVHTMSSSFNTVKNYVTLGGAIWDEQTQKGLGILQSIWHLLLQEPEGISDNGFDKPVNDSSMLCQKLYFSFELRPGTDFPQVKTYVPTWNYLRTDGETIQNYEAIFRACDHPWGEDRTYGKIFQDAFGPATESRKKPIHCDASFLFTEETGVYQTLYFSPPIEGETEVQSNLVA

SEQ ID NO: 20 - DNA, 2063 bp, Epichloe festucae
atgcaatacggtaatttaacaactgtattacttctgcgtaatactttattgtccttgaattcttcgtcaatctgccatgttcactggctgcaagtgattgtggctctgcttgtcttgatcgtctgcatctttctatattggcgaacacccactggcatcaatgctcctttcgcaggatatcgttcaccatgggagccgccgctcttggttcagatgcgttacgtcttcaacgctgcctcaatgatacgcgaaggatatgctaaggtatgttttatcccgcgtagaggtcttctacccggatagaccgagaagataacaacttcggaacagtggaaagactccttgttccagatctcacgatacgacggtgacattcttattgtgcctccaagatatttggatgacctccacaacaagtcacaagaggagttaagtgctatttatggtttgattcgggtgaggaatgccaccaaccaaaaaacgcagagcctattagcgcatggtctcacatattcgaatttgctagaattttggtggtagctatagcggcatcaccctgcttggagaaaacgatgttggcattcgtgcgcttcaggtatgtacacccttccaaaagtctgttagggaccttccttattctactacagacaaaaatcaccccaaatcttgcgaaattatgcgatgacataagggatgagtttcagtattgtctagatacagacttcccagcctgcagaggtatgccatttccaaaatcccattatgcagtctctactttttctggcactaacgatatctaacatagattggacatcagtgtccgtgcatccattgtttctaaaagcagtcgaaaggataacacatcggatttttgttggattgccattatgtcggaatccccaatgggtccaagcgaccagcaagcatgcacattacggtacgtcaattgactaataataggcaatatacgcgctcatatgctttgcagcaacaatgatacagatagctatgagatctgtcccaaagttcattcagcctttactaaatttttgccttccgtggccatggaagaacgcagcctgtgttcgtgaagcaaagaatgcccttatattagaaatgcaacgccgacgaaatctcgagaaagttaacagttttgattatatcaaatccaatgacttgctgcaagcagttatggaaatgtcttctcctagtcatgaggatagccagcttgatgttgtcgcccagataatgctcacgatgaacacaatcgctggccacagtactgccgcatccggagcacatgcactgttcgatatggttagccactctaagtatattgaattgctgcgtgaggaggctcttcaagtctttcgacatgttgaactgcgtgttacaaaacaggctttgggggatttgcgaaaattggacagcttcctcagagagttagtattgtcctaaacatcacaatctcaccacattctcacgctagcttttcctccgtactaatgatggtcgttgctaagatcccaacgacataatccgctaagcttgtgtatgtttagctaagagtctcgaaaacctggaaatgtttgtcctgtgcccgagttctaacgtctcttactacagtaggcttttttcgggtcgtattagaccctgccggtatcacacttcaagatggcacacatgttccttacaacacactgctttgtgtcgcaccacatgcgatatccaatgacccggatgtgatagaagacccaacctcgttcaacggtctgcgatactacgaacagcgctgtcgtgacgccagtcaagagaaaaagcatcaatacgctactacggataaatctcacctgcattttggctacggaacctgggcctgtccaggccgcttcttggcctctgatatgttaaaagtgattctaacgatgcttctgcttcagtatgacatccgctcccccgagagagcaaaacggcctgtggcaggtcattttcatgagtttccgcttttcaatattaacacaccactgttaatgaaacgacgcaatgattcgctagttctatga

SEQ ID NO: 21 - protein, 503 aa, Epichloe festucae
  DOMAIN (353)..(487): cytochrome P450 domain
MQYGNLTTVLLLRNTLLSLNSSSICHVHWLQVIVALLVLIVCIFLYWRTPTGINAPFAGYRSPWEPPLLVQMRYVFNAASMIREGYAKWKDSLFQISRYDGDILIVPPRYLDDLHNKSQEELSAIYGLIRNFGGSYSGITLLGENDVGIRALQTKITPNLAKLCDDIRDEFQYCLDTDFPACRDWTSVSVHPLFLKAVERITHRIFVGLPLCRNPQWVQATSKHAHYATMIQIAMRSVPKFIQPLLNFCLPWPWKNAACVREAKNALILEMQRRRNLEKVNSFDYIKSNDLLQAVMEMSSPSHEDSQLDVVAQIMLTMNTIAGHSTAASGAHALFDMLRVSKTWKCLSCARVLTSLTTVGFFRVVLDPAGITLQDGTHVPYNTLLCVAPHAISNDPDVIEDPTSFNGLRYYEQRCRDASQEKKHQYATTDKSHLHFGYGTWACPGRFLASDMLKVILTMLLLQYDIRSPERAKRPVAGHFHEFPLFNINTPLLMKRRNDSLVL

SEQ ID NO: 22 - DNA, 2063 bp, Epichloe festucae
atgcaatacggtaatttaacaactgtattacttctgcgtaatactttattgtccttgaattcttcgtcaatctgccatgttcactggctgcaagtgattgtggctctgcttgtcttgatcgtctgcatctttctatattggcgaacacccactggcatcaatgctcctttcgcaggatatcgttcaccatgggagccgccgctcttggttcagatgcgttacgtcttcaacgctgcctcaatgatacgcgaaggatatgctaaggtatgttttatcccgcgtagaggtcttctacccggatagaccgagaagataacaacttcggaacagtggaaagactccttgttccagatctcacgatacgacggtgacattcttattgtgcctccaagatatttggatgaccttcacaacaagtcacaagagaagttaagtgctatttatggtttgattcgggtgaggaatgccaccaaccaaaaaacgcagagcctattagcgcatggtctcacatattcgaatttgctagaattttggtggtagctatagcggcattaccctgcttggagaaaacgatgttggcattcgtgcgcttcaggtatgtacacccttccaaaagtctgttagggaccttccttactctactacagacgaaaatcaccccaaatcttgcgaaattatgcgatgacataagggatgagtttcagtattgtctagatacagacttcccagcctgcagaggtatgccatttccaaaatcccattatgcagtctctactttttctggcactaacgatatctaacatagattggacatcagtgtccgtgcatccattgtttctaaaagcagtcgaaaggataatacatcggatttttgttggattgccattatgtcggaatccccaatgggtccaagcgaccagcaagcatgcacattacggtacgtcaattgactaataataggcaatatacgcgctcatatgctttgcagcaacaatgatacagatagctatgagatctgtcccaaagttcattcagcctttactaaatttttgccttccgtggccatggaagaacgcagcctgtgttcgtgaagcaaagaatgcccttataatagaaatgcaacgccgacgaaatctcgagaaagttaacagttttgattatatcaaatccaatgacttgctgcaagcagttatggaaatgtcttctcctagtcatgaggatagccagcttgatgttgtcgcccagataatgctcacgatgaacaatatcgctggccacagtactgccgcatccggagcacatgcactgttcgatatggttagccactctaagtatattgaattgctgcgtgaggaggctcttcaagtctttcgacatgttgaactgcgtgttacaaaacaggctttgggggatttgcgaaaattggacagcttcctcagagagttagtattgtcctaaacatcacaatctcaccacattctcacgctagcttttcctccgtactaatgatggtcgttgctaagatcccaacgacataatccgctaagcttgtgtatgtttagctaagagtctcgaaaacctggaaatgtttgtcctgtgcccgagttctaacgtctcttactacagtaggcttttttcgggtcgtattagaccctgccggtatcacacttcaagatggcacacatgttccttacaacacactgctttgtgtcgcaccacatgcgatatccaatgacccggatgtgatagaagacccaacctcgttcaacggtctgcgatactacgaacagcgctgtcgtgacgccagtcaagagaaaaagcatcaatacgctactacggataaatctcacctgcattttggctacggaacctgggcctgtccaggccgcttcttggcctctgatatgttaaaagtgattctaacgatgcttctgcttcagtatgacatccgctcccccgagagagcaaaacggcctgtggcaggtcattttcatgagtttccgcttttcaatattaacacaccactgttaatgaaacgacgcaatgattcgctagttctatga

SEQ ID NO: 23 - protein, 503 aa, Epichloe festucae
  DOMAIN (353)..(487): cytochrome P450 domain
MQYGNLTTVLLLRNTLLSLNSSSICHVHWLQVIVALLVLIVCIFLYWRTPTGINAPFAGYRSPWEPPLLVQMRYVFNAASMIREGYAKWKDSLFQISRYDGDILIVPPRYLDDLHNKSQEKLSAIYGLIRNFGGSYSGITLLGENDVGIRALQTKITPNLAKLCDDIRDEFQYCLDTDFPACRDWTSVSVHPLFLKAVERIIHRIFVGLPLCRNPQWVQATSKHAHYATMIQIAMRSVPKFIQPLLNFCLPWPWKNAACVREAKNALIIEMQRRRNLEKVNSFDYIKSNDLLQAVMEMSSPSHEDSQLDVVAQIMLTMNNIAGHSTAASGAHALFDMLRVSKTWKCLSCARVLTSLTTVGFFRVVLDPAGITLQDGTHVPYNTLLCVAPHAISNDPDVIEDPTSFNGLRYYEQRCRDASQEKKHQYATTDKSHLHFGYGTWACPGRFLASDMLKVILTMLLLQYDIRSPERAKRPVAGHFHEFPLFNINTPLLMKRRNDSLVL

SEQ ID NO: 24 - DNA, 23 bp, Artificial
  synthetic oligonucleotide synthesised in laboratory
ggaagagcttagatacaaagtgg

SEQ ID NO: 25 - DNA, 23 bp, Artificial
  synthetic oligonucleotide synthesised in laboratory
cttcctggatcgagatacggcgg

SEQ ID NO: 26 - DNA, 23 bp, Artificial
  synthetic oligonucleotide synthesised in laboratory
gcatggcctcgggaaggtgaggg

SEQ ID NO: 27 - DNA, 23 bp, Artificial
  synthetic oligonucleotide synthesised in laboratory
ggagccagcgatacaaagggcgg

SEQ ID NO: 28 - DNA, 39 bp, Artificial
  synthetic oligonucleotide synthesised in laboratory
ctttgtatctaagctcttccgacgagcttactcgtttcg

SEQ ID NO: 29 - DNA, 43 bp, Artificial
  synthetic oligonucleotide synthesised in laboratory
ggaagagcttagatacaaaggttttagagctagaaatagcaag

SEQ ID NO: 30 - DNA, 43 bp, Artificial
  synthetic oligonucleotide synthesised in laboratory
cttcctggatcgagatacgggttttagagctagaaatagcaag

SEQ ID NO: 31 - DNA, 39 bp, Artificial
  synthetic oligonucleotide synthesised in laboratory
ccgtatctcgatccaggaaggacgagcttactcgtttcg

SEQ ID NO: 32 - DNA, 23 bp, Artificial
  synthetic oligonucleotide synthesised in laboratory
gtcggagccagcgatacaaaggg

SEQ ID NO: 33 - DNA, 23 bp, Artificial
  synthetic oligonucleotide synthesised in laboratory
aacccctttgtatcgctggctcc

SEQ ID NO: 34 - DNA, 23 bp, Artificial
  synthetic oligonucleotide synthesised in laboratory
gtcgcatggcctcgggaaggtga

SEQ ID NO: 35 - DNA, 23 bp, Artificial
  synthetic oligonucleotide synthesised in laboratory
aactcaccttcccgaggccatgc

SEQ ID NO: 36 - DNA, 23 bp, Artificial
  synthetic oligonucleotide synthesised in laboratory
ggggatcataatagtactagcca

SEQ ID NO: 37 - DNA, 19 bp, Artificial
  synthetic oligonucleotide synthesised in laboratory
ttttctcttccatttacgc

SEQ ID NO: 38 - DNA, 20 bp, Artificial
  synthetic oligonucleotide synthesised in laboratory
accccaagattgcatcccag

SEQ ID NO: 39 - DNA, 20 bp, Artificial
  synthetic oligonucleotide synthesised in laboratory
ggcggtgatctgttctggaa

SEQ ID NO: 40 - DNA, 20 bp, Artificial
  synthetic oligonucleotide synthesised in laboratory
gggacagacgttagcactcc

SEQ ID NO: 41 - DNA, 20 bp, Artificial
  synthetic oligonucleotide synthesised in laboratory
ccctaccgtgctgaagagtg

SEQ ID NO: 42 - DNA, 29 bp, Artificial
  synthetic oligonucleotide synthesised in laboratory
gccggtaccgcgcttacacagtacacgag

SEQ ID NO: 43 - DNA, 38 bp, Artificial
  synthetic oligonucleotide synthesised in laboratory
aaggaaaaaagcggccgcctaaaatccgccgcctccac

SEQ ID NO: 44 - DNA, 47 bp, Artificial
  synthetic oligonucleotide synthesised in laboratory
gtcggaagagcaaaatgctcttcagttttagagctagaaatagcaag

SEQ ID NO: 45 - DNA, 43 bp, Artificial
  synthetic oligonucleotide synthesised in laboratory
aactgaagagcattttgctcttccgacgagcttactcgtttcg

SEQ ID NO: 46 - DNA, 38 bp, Artificial
  synthetic oligonucleotide synthesised in laboratory
caacctccaatccaatttgactccgccgaacgtactgg

SEQ ID NO: 47 - DNA, 48 bp, Artificial
  synthetic oligonucleotide synthesised in laboratory
actactctaccactatttgaaaagcaaaaaaggaaggtacaaaaaagc

SEQ ID NO: 48 - DNA, 38 bp, Artificial
  synthetic oligonucleotide synthesised in laboratory
tcggctcgtcggaagagcgaccgacagccttccaaatg

SEQ ID NO: 49 - DNA, 38 bp, Artificial
  synthetic oligonucleotide synthesised in laboratory
tcgctaaaactgaagagcgttggcagcatcacccgacg

SEQ ID NO: 50 - protein, 200 aa, Epichlo festucae var. lolii strain AR37
RNVQVALILAQPDVARFTGLETSSDGQSLIRNPFARPTKMGTCSTRVGETPSKPADVTPPEPWQALAQGLGFANENERYWWSKLAPLAGKMMKWGQYSTPEQYRVLAFIHAYIVPSCGPRPGDGGDLFWKVFLNYDCTPIQLSLNYHDGKMTLRTAHIPISNISGTAEDPINQKAAIDAMVRQQQVLPSQDMRWFNHFCI

SEQ ID NO: 51 - protein, 240 aa, Epichlo festucae var. lolii strain AR37
RNVQVALILAQPDVARFTGLETSSDGQSLIRNPFARPTKMGTCSTRVGETPSKPADVTPPEPWQALAQGLGFANENERYWWSKLAPLAGKMMKWGQYSTPEQYRVLAFIHAYIVPSCGPRPGDGGDLFWKVFLNYDCTPIQLSLNYHDGKMTLRTAHIPISNISGTAEDPINQKAAIDAMVRQQQVLPSQDMRWFNHFVSKLFLDRDNGGHPQGQGGRVPDPAGSSVHAQPRLSRQSHPV

SEQ ID NO: 52 - protein, 75 aa, Epichlo festucae var. lolii strain AR37
MLVIERDIYADYYELDDKEKTPVRYRSLSSWGKWEWCLAHCFSARGIGFSWAIPHPSRGHAQQHDDSGLPAGERS

SEQ ID NO: 53 - protein, 253 aa, Epichlo festucae var. lolii strain AR37
MLVIERDIYADYYELDDKEKTPVRYRSLSSWGKWEWCLAHCFSARGIGFSWAIPHLPEAMPSNTTIRDYLRASALNLGWLYLVQDLGRSLLSADLFAHEGVGASDTGRRPFSDRLFTWNRGLVEHRHAIPCCMRHGHGQRVLLDQAAPQPACSRPLAGRMDAATLLGPRLAPDVPQALAVDRSMDCLGSHARPQRLPCLEIRPGLHFFSAIGLDARCRCADGRSAPTLLRRDLDLLSHAGQRHRGRGCCAVGW

SEQ ID NO: 54 - DNA, 23 bp, Artificial
  synthetic oligonucleotide synthesised in laboratory
agagactgtggccacctgagcgg

SEQ ID NO: 55 - DNA, 23 bp, Artificial
  synthetic oligonucleotide synthesised in laboratory
tcaaccagtacgatgaagcacgg

SEQ ID NO: 56 - DNA, 23 bp, Artificial
  synthetic oligonucleotide synthesised in laboratory
tgtaagataggatggcccagtgg

SEQ ID NO: 57 - DNA, 23 bp, Artificial
  synthetic oligonucleotide synthesised in laboratory
attcaaggcttcgtacctgaggg

SEQ ID NO: 58 - DNA, 23 bp, Artificial
  synthetic oligonucleotide synthesised in laboratory
gtcagagactgtggccacctgag

SEQ ID NO: 59 - DNA, 23 bp, Artificial
  synthetic oligonucleotide synthesised in laboratory
aacctcaggtggccacagtctct

SEQ ID NO: 60 - DNA, 23 bp, Artificial
  synthetic oligonucleotide synthesised in laboratory
gtctcaaccagtacgatgaagca

SEQ ID NO: 61 - DNA, 23 bp, Artificial
  synthetic oligonucleotide synthesised in laboratory
aactgcttcatcgtactggttga

SEQ ID NO: 62 - DNA, 23 bp, Artificial
  synthetic oligonucleotide synthesised in laboratory
gtctgtaagataggatggcccag

SEQ ID NO: 63 - DNA, 23 bp, Artificial
  synthetic oligonucleotide synthesised in laboratory
aacctgggccatcctatcttaca

SEQ ID NO: 64 - DNA, 23 bp, Artificial
  synthetic oligonucleotide synthesised in laboratory
gtcattcaaggcttcgtacctga

SEQ ID NO: 65 - DNA, 23 bp, Artificial
  synthetic oligonucleotide synthesised in laboratory
aactcaggtacgaagccttgaat

SEQ ID NO: 66 - DNA, 20 bp, Artificial
  synthetic oligonucleotide synthesised in laboratory
cggcccaatacaaatcagcg

SEQ ID NO: 67 - DNA, 20 bp, Artificial
  synthetic oligonucleotide synthesised in laboratory
gtgctgtcccggattgaaga

SEQ ID NO: 68 - DNA, 20 bp, Artificial
  synthetic oligonucleotide synthesised in laboratory
ccctttcgctcgctctcttt

SEQ ID NO: 69 - DNA, 20 bp, Artificial
  synthetic oligonucleotide synthesised in laboratory
cagattgcacccccaagagt

SEQ ID NO: 70 - protein, 81 aa, Epichlo festucae var. lolii strain AR37
MRDSSTEKLPILWRTDSPLNQYDEARCRVFNGRRPEHFPRAIVQATTLDHIVAAVRLAVESAAPVAVRSRWPQSLLLDHAP

SEQ ID NO: 71 - protein, 81 aa, Epichlo festucae var. lolii strain AR37
MRDSSTEKLPILWRTDSPLNQYDESTVQSLQRQAARAFPTRNRPGHDARPHRSGCEAGRGVRRPCGRPLRWPQSLLLDHAP

SEQ ID NO: 72 - protein, 80 aa, Epichlo festucae var. lolii strain AR37
MIAKNIELNGLDPATRALDILYWKNHCIKQLESLLCATDSYCTADKAAQLRILSELVLPNLGPRPSNATGHPILHEVVPQ

SEQ ID NO: 73 - protein, 179 aa, Epichlo festucae var. lolii strain AR37
MIAKNIELNGLDPATRALDILYWKNHCIKQLESLLCATDSYCTADKAAQLRILSELVLPNLGPRPSNATGPSYLTRSGSPIMLSLNTTSSKNCVRYCWEILGATGASNDDPLAVQVAKDVVASLSATFRLSTKWSETLLSNFAVTPDQARQVINMLPEWIQGFVLREWSAIFQRESRSP

SEQ ID NO: 74 - protein, 165 aa, Epichlo festucae var. lolii strain AR37
MIAKNIELNGLDPATRALDILYWKNHCIKQLESLLCATDSYCTADKAAQLRILSELVLPNLGPRPSNATGPSYLTRSGSPIMLSLNTTSSKNCVRYCWEILGATGASNDDPLAVQVAKDVVASLSATFRLSTKWSETLLSNFAVTPDQARQVINMLPEWIQGFVP

(19) United States
(12) Patent Application Publication
(10) Pub. No.: US 2023/0145729 A1
(43) Pub. Date: May 11, 2023

(54) INDOLE DITERPENE BIOSYNTHESIS

(71) Applicant: GRASSLANZ TECHNOLOGY LIMITED, Lincoln (NZ)

(72) Inventors: Alison Jean POPAY, Lincoln (NZ); Debbie Anne HUDSON, Lincoln (NZ); Richard David JOHNSON, Lincoln (NZ); Sarah Christine FINCH, Lincoln (NZ); Wade Jeffery MACE, Lincoln (NZ)

(21) Appl. No.: 17/995,191

(22) PCT Filed: Mar. 31, 2021
(86) PCT No.: PCT/NZ2021/050055

§ 371 Date: Sep. 30, 2022

(30) Foreign Application Priority Data
Mar. 31, 2020 (NZ) 763153
Jun. 23, 2020 (NZ) 765613

(51) Int. Cl. C12N 15/80; C07K 14/37; C12N 1/14; C12N 15/52; C12P 17/18

(52) U.S. Cl. CPC C12N 15/80; C07K 14/37; C12N 1/145; C12N 15/52; C12P 17/188; C12R 2001/645

(57) ABSTRACT

The present invention generally relates to methods of producing one or more indole diterpene compounds in the epoxy janthitrem biosynthetic pathway and polynucleotides, polypeptides, expression constructs and host cells useful in the production of one or more indole diterpene compounds, and methods of using same to confer a benefit to an organism, such as increased insect pest resistance.

### TECHNICAL FIELD

The invention generally relates to the biosynthesis of certain indole diterpenes, for example in a heterologous host, together with polynucleotides, polypeptides, and related methods for synthesising said indole diterpenes, for example by expression in a heterologous host. The invention further relates to heterologous hosts capable of synthesising said indole diterpene compounds.

### BACKGROUND OF THE INVENTION

The following includes information that may be useful in understanding the present invention. It is not an admission that any of the information provided herein is prior art, or relevant, to the presently described or claimed invention, or that any publication or document that is specifically or implicitly referenced is prior art. Any discussion of the prior art throughout the specification should in no way be considered as an admission that such prior art is widely known or forms part of the common general knowledge in the field in any country.

Fungal secondary metabolites are of interest principally due to their wide-ranging bioactivities including, for example, insecticidal and pesticidal activities, and mammalian tremorgenicity, and the application of these activities in medicine, agriculture and horticulture. For example, the degree of plant productivity, persistence, and pest resistance observed in and required of many modern agricultures has been attributed to the presence of endophytic fungi, such as Epichloë, and the production by such endophytes of one or more secondary metabolites.

For example, in New Zealand, protection against Argentine stem weevil (Listronotus bonariensis) and African black beetle (Heteronychus arator), the key pests of the most widely grown pasture grass species perennial ryegrass (Lolium perenne L.), is observed in the presence of Epichloë endophytic fungi and is driven by the effect of the secondary metabolites peramine, and ergovaline, respectively. Other insecticidal secondary metabolites, such as the indole diterpenes including for example the janthitrems and epoxy-janthitrems, have also been associated with insect pest protection in Epichloë-infected pasture grasses.

Indole diterpenes are a large, structurally diverse group of compounds found in filamentous fungi, including fungi of the genera Penicillium, Aspergillus, Claviceps, and Epichloë (formerly Neotyphodium). Indole diterpenes have been classified into the following sub-groups: penitrems, janthitrems, sulpinines, nodulisporic acids, thiersinines, terpendoles, shearinines, aflatrem, paxilline and analogues, emindoles, and lolitrems, all of which have a common core structure comprising a cyclic diterpene skeleton derived from geranylgeranyl diphosphate (GGPP) and an indole moiety derived from either tryptophan or a tryptophan precursor.

However, in addition to beneficial and desirable effects, certain indole diterpenes have been associated with undesirable bioactivities. For example, many naturally occurring endophyte-perennial ryegrass associations also expresses lolitrem B, a secondary metabolite which is responsible for ryegrass staggers, a neurological condition of grazing animals which causes major production losses and farm management issues.

Consequently, there is a need for ways of providing one or more beneficial indole diterpene compounds, without concomitant or with reduced provision of one or more detrimental or undesirable indole diterpene compounds.

It is therefore an object of the invention to provide one or more host cells capable of producing one or more epoxy-janthitrem compounds and/or one or more plants comprising said one or more host cells, and/or to provide a method of conferring a beneficial effect on a plant, and/or to provide a polynucleotide capable of expressing a polypeptide and/or a polypeptide involved in the biosynthesis of one or more epoxy-janthitrem compounds or one or more janthitrem compounds, or to at least provide a useful alternative to existing methods, or to at least provide the public with a useful choice.

All references, including any patents or patent applications cited in this specification, are hereby incorporated by reference in their entirety. The discussion of the references states what their authors assert, and the applicants reserve the right to challenge the accuracy and pertinency of the cited documents.

### SUMMARY OF THE INVENTION

In a first aspect the invention relates to a host cell comprising a polypeptide selected from the group consisting of:
a. a polypeptide comprising, consisting essentially of, or consisting of an amino acid sequence corresponding to the amino acid sequence of IdtA from Epichloë festucae var. lolii strain AR37, or corresponding to a polypeptide encoded by the idtA gene from Epichloë festucae var. lolii strain AR37;
b. a polypeptide comprising, consisting essentially of, or consisting of an amino acid sequence corresponding to the amino acid sequence of IdtD from Epichloë festucae var. lolii strain AR37, Epichloë festucae var. lolii strain AR127, Epichloë festucae var. lolii strain AR128, or Epichloë festucae var. lolii strain AR166, or corresponding to a polypeptide encoded by the idtD gene from Epichloë festucae var. lolii strain AR37, Epichloë festucae var. lolii strain AR127, Epichloë festucae var. lolii strain AR128, or Epichloë festucae var. lolii strain AR166;
c. a polypeptide comprising, consisting essentially of, or consisting of an amino acid sequence corresponding to the amino acid sequence of IdtO from Epichloë festucae var. lolii strain AR37, or corresponding to a polypeptide encoded by the idtO gene from Epichloë festucae var. lolii strain AR37;
d. a polypeptide comprising, consisting essentially of, or consisting of an amino acid sequence corresponding to the amino acid sequence of IdtF from Epichloë festucae var. lolii strain AR37 or Epichloë festucae var. lolii strain AR6, or corresponding to a polypeptide encoded by the idtF gene from Epichloë festucae var. lolii strain AR37 or Epichloë festucae var. lolii strain AR6;
e. a polypeptide comprising, consisting essentially of, or consisting of an amino acid sequence corresponding to the amino acid sequence of IdtK from Epichloë festucae var. lolii strain AR37 or Epichloë festucae var. lolii strain AR6, or corresponding to a polypeptide encoded by the idtK gene from Epichloë festucae var. lolii strain AR37 or Epichloë festucae var. lolii strain AR6;
f. a polypeptide comprising, consisting essentially of, or consisting of an amino acid sequence set forth in any one of SEQ ID NO: 3, 6, 9, 17, 19, 21, 23, 50-53, or 70-74; or
g. a polypeptide comprising, consisting essentially of, or consisting of an amino acid sequence corresponding to at least 10 contiguous amino acids from an amino acid sequence set forth in any one of SEQ ID NO: 3, 6, 9, 17, 19, 21, 23, 50-53, or 70-74;
h. a polypeptide comprising, consisting essentially of, or consisting of an amino acid sequence corresponding to amino acid residues 150 to 239 of SEQ ID NO: 3; or
i. a polypeptide comprising, consisting essentially of, or consisting of an amino acid sequence corresponding to at least 10 contiguous amino acids from an amino acid sequence set forth in amino acid residues 150 to 239 of SEQ ID NO: 3;
j. a polypeptide comprising, consisting essentially of, or consisting of an amino acid sequence corresponding to amino acid residues 76 to 436 of SEQ ID NO: 6; or
k. a polypeptide comprising, consisting essentially of, or consisting of an amino acid sequence corresponding to at least 10 contiguous amino acids from an amino acid sequence set forth in amino acid residues 76 to 436 of SEQ ID NO: 6;
l. a polypeptide comprising, consisting essentially of, or consisting of an amino acid sequence corresponding to amino acid residues 39 to 174 of SEQ ID NO: 9; or
m. a polypeptide comprising, consisting essentially of, or consisting of an amino acid sequence corresponding to at least 10 contiguous amino acids from an amino acid sequence set forth in amino acid residues 39 to 174 of SEQ ID NO: 9;
n. a polypeptide comprising, consisting essentially of, or consisting of an amino acid sequence corresponding to amino acid residues 19 to 386 of SEQ ID NO: 17 or 19; or
o. a polypeptide comprising, consisting essentially of, or consisting of an amino acid sequence corresponding to at least 10 contiguous amino acids from an amino acid sequence set forth in amino acid residues 19 to 386 of SEQ ID NO: 17 or 19;
p. a polypeptide comprising, consisting essentially of, or consisting of an amino acid sequence corresponding to amino acid residues 353 to 487 of SEQ ID NO: 21 or 23; or
q. a polypeptide comprising, consisting essentially of, or consisting of an amino acid sequence corresponding to at least 10 contiguous amino acids from an amino acid sequence set forth in amino acid residues 353 to 487 of SEQ ID NO: 21 or 23;
r. a polypeptide comprising, consisting essentially of, or consisting of an amino acid sequence encoded by a polynucleotide sequence set forth in any one of SEQ ID NO: 1, 2, 4, 5, 7, 8, 10 to 16, 18, 20, or 22;
s. a polypeptide encoded by a polynucleotide sequence having at least about 90% nucleic acid sequence identity to a sequence set forth in any one of SEQ ID NO: 1, 2, 4, 5, 7, 8, 10 to 16, 18, 20, or 22;
t. a polypeptide involved in the production of one or more janthitrem compound, including a janthitrem compound of formulae VI, or an epoxy-janthitrem compound of any one of formulae I to III or V to VIII;
u. a polypeptide comprising an enzymatic activity having as its substrate terpendole I or as its substrate or its product a compound of any one of formulae Ito VIII, such as a compound of any one of formulae IIA to IIE;
v. a catalytically active fragment of any one of a) to u) above; and
w. a polypeptide comprising, consisting essentially of, or consisting of a sequence of amino acid residues that has at least 70%, at least 75%, at least 80%, at least 85%, or at least 90% or greater amino acid sequence identity to any one of a) to t) above; or
x. any combination of two or more of a) to w) above;

wherein the polypeptide is heterologous to the cell or is heterologously expressed by the cell.

In one embodiment, the polypeptide is a polypeptide comprising, consisting essentially of, or consisting of:
a. an amino acid sequence set forth in SEQ ID NO: 3; or
b. a polypeptide comprising, consisting essentially of, or consisting of a sequence of amino acid residues that has at least 90% or greater amino acid sequence identity to the amino acid sequence set forth in SEQ ID NO: 3; or
c. a polypeptide comprising, consisting essentially of, or consisting of an amino acid sequence corresponding to amino acid residues 150 to 239 of SEQ ID NO: 3; or
d. a polypeptide comprising, consisting essentially of, or consisting of a sequence of amino acid residues that has at least 90% or greater sequence identity to the amino acid sequence corresponding to amino acid residues 150 to 239 of SEQ ID NO: 3; or
e. a polypeptide comprising, consisting essentially of, or consisting of an amino acid sequence corresponding to at least 10 contiguous amino acids from an amino acid sequence set forth in SEQ ID NO: 3;
f. a polypeptide comprising, consisting essentially of, or consisting of an amino acid sequence corresponding to at least 10 contiguous amino acids from an amino acid sequence set forth in amino acid residues 150 to 239 of SEQ ID NO: 3;
g. a polypeptide encoded by a polynucleotide sequence having at least about 70%, at least 75%, at least 80%, at least 85%, or at least about 90% nucleic acid sequence identity to a sequence set forth in any one of SEQ ID NO: 1 or 2;
h. a catalytically active fragment of any one of a) to g); or
i. a polypeptide that catalyses the conversion of a substrate in the epoxy-janthitrem biosynthetic pathway from terpendole I onwards.

In one embodiment, the polypeptide is a polypeptide comprising, consisting essentially of, or consisting of:
a. an amino acid sequence set forth in SEQ ID NO: 6; or
b. a polypeptide comprising, consisting essentially of, or consisting of a sequence of amino acid residues that has at least 90% or greater amino acid sequence identity to the amino acid sequence set forth in SEQ ID NO: 6; or
c. a polypeptide comprising, consisting essentially of, or consisting of an amino acid sequence corresponding to amino acid residues 76 to 436 of SEQ ID NO: 6; or
d. a polypeptide comprising, consisting essentially of, or consisting of a sequence of amino acid residues that has at least 90% or greater sequence identity to the amino acid sequence corresponding to amino acid residues 76 to 436 of SEQ ID NO: 6; or
e. a polypeptide comprising, consisting essentially of, or consisting of an amino acid sequence corresponding to at least 10 contiguous amino acids from an amino acid sequence set forth in SEQ ID NO: 6;
f. a polypeptide comprising, consisting essentially of, or consisting of an amino acid sequence corresponding to at least 10 contiguous amino acids from an amino acid sequence set forth in amino acid residues 76 to 436 of SEQ ID NO: 6;
g. a polypeptide encoded by a polynucleotide sequence having at least about 70%, at least 75%, at least 80%, at least 85%, or at least about 90% nucleic acid sequence identity to a sequence set forth in any one of SEQ ID NO: 4 or 5;
h. a catalytically active fragment of any one of a) to g); or
i. a polypeptide that catalyses the conversion of a substrate in the epoxy-janthitrem biosynthetic pathway from terpendole I onwards.

In one embodiment, the polypeptide is a polypeptide comprising, consisting essentially of, or consisting of:
a. an amino acid sequence set forth in SEQ ID NO: 9; or
b. a polypeptide comprising, consisting essentially of, or consisting of a sequence of amino acid residues that has at least 90% or greater amino acid sequence identity to the amino acid sequence set forth in SEQ ID NO: 9; or
c. a polypeptide comprising, consisting essentially of, or consisting of an amino acid sequence corresponding to amino acid residues 39 to 174 of SEQ ID NO: 9; or
d. a polypeptide comprising, consisting essentially of, or consisting of a sequence of amino acid residues that has at least 90% or greater sequence identity to the amino acid sequence corresponding to amino acid residues 39 to 174 of SEQ ID NO: 9; or
e. a polypeptide comprising, consisting essentially of, or consisting of an amino acid sequence corresponding to at least 10 contiguous amino acids from an amino acid sequence set forth in SEQ ID NO: 9;
f. a polypeptide comprising, consisting essentially of, or consisting of an amino acid sequence corresponding to at least 10 contiguous amino acids from an amino acid sequence set forth in amino acid residues 39 to 174 of SEQ ID NO: 9;
g. a polypeptide encoded by a polynucleotide sequence having at least about 70%, at least 75%, at least 80%, at least 85%, or at least about 90% nucleic acid sequence identity to a sequence set forth in any one of SEQ ID NO: 7 or 8;
h. a catalytically active fragment of any one of a) to g); or
i. a polypeptide that catalyses the conversion of a substrate in the epoxy-janthitrem biosynthetic pathway from terpendole I onwards.

In one embodiment, the polypeptide is a polypeptide comprising, consisting essentially of, or consisting of:
a. an amino acid sequence set forth in SEQ ID NO: 17 or 19; or
b. a polypeptide comprising, consisting essentially of, or consisting of a sequence of amino acid residues that has at least 90% or greater amino acid sequence identity to the amino acid sequence set forth in SEQ ID NO: 17 or 19; or
c. a polypeptide comprising, consisting essentially of, or consisting of an amino acid sequence corresponding to amino acid residues 19 to 386 of SEQ ID NO: 17 or 19; or
d. a polypeptide comprising, consisting essentially of, or consisting of a sequence of amino acid residues that has at least 90% or greater sequence identity to the amino acid sequence corresponding to amino acid residues 19 to 386 of SEQ ID NO: 17 or 19; or
e. a polypeptide comprising, consisting essentially of, or consisting of an amino acid sequence corresponding to at least 10 contiguous amino acids from an amino acid sequence set forth in SEQ ID NO: 6;
f. a polypeptide comprising, consisting essentially of, or consisting of an amino acid sequence corresponding to at least 10 contiguous amino acids from an amino acid sequence set forth in amino acid residues 19 to 386 of SEQ ID NO: 17 or 19;
g. a polypeptide encoded by a polynucleotide sequence having at least about 70%, at least 75%, at least 80%, at least 85%, or at least about 90% nucleic acid sequence identity to a sequence set forth in any one of SEQ ID NO: 16 or 18;
h. a catalytically active fragment of any one of a) to g); or
i. a polypeptide that catalyses the conversion of a substrate in the epoxy-janthitrem biosynthetic pathway from terpendole I onwards.

In one embodiment, the polypeptide is a polypeptide comprising, consisting essentially of, or consisting of:
a. an amino acid sequence set forth in SEQ ID NO: 21 or 23; or
b. a polypeptide comprising, consisting essentially of, or consisting of a sequence of amino acid residues that has at least 90% or greater amino acid sequence identity to the amino acid sequence set forth in SEQ ID NO: 21 or 23; or
c. a polypeptide comprising, consisting essentially of, or consisting of an amino acid sequence corresponding to amino acid residues 353 to 487 of SEQ ID NO: 21 or 23; or
d. a polypeptide comprising, consisting essentially of, or consisting of a sequence of amino acid residues that has at least 90% or greater sequence identity to the amino acid sequence corresponding to amino acid residues 353 to 487 of SEQ ID NO: 21 or 23; or
e. a polypeptide comprising, consisting essentially of, or consisting of an amino acid sequence corresponding to at least 10 contiguous amino acids from an amino acid sequence set forth in SEQ ID NO: 6;
f. a polypeptide comprising, consisting essentially of, or consisting of an amino acid sequence corresponding to at least 10 contiguous amino acids from an amino acid sequence set forth in amino acid residues 353 to 487 of SEQ ID NO: 20 or 22;
g. a polypeptide encoded by a polynucleotide sequence having at least about 70%, at least 75%, at least 80%, at least 85%, or at least about 90% nucleic acid sequence identity to a sequence set forth in any one of SEQ ID NO: 20 or 22;
h. a catalytically active fragment of any one of a) to g); or
i. a polypeptide that catalyses the conversion of a substrate in the epoxy-janthitrem biosynthetic pathway from terpendole I onwards.

In another aspect, the present invention relates to an isolated, purified, recombinant, or synthesised polypeptide as defined herein.

In another aspect, the present invention relates to an isolated, purified, recombinant, or synthesised polynucleotide encoding a polypeptide as defined herein.

In a further aspect, the present invention relates to an isolated, purified, recombinant, or synthesised polynucleotide comprising at least about 70%, at least 75%, at least 80%, at least 85%, or at least about 90% nucleic acid sequence identity to the nucleic acid sequence set forth in any one of SEQ ID NO: 1, 2, 4, 5, 7, 8, 10 to 16, 18, 20, or 22.

In another aspect, the present invention relates to a polynucleotide comprising a nucleic acid sequence encoding a polypeptide and a transcription control sequence, wherein the polypeptide comprises an amino acid sequence at least about 70%, at least 75%, at least 80%, at least 85%, at least 90%, or at least about 95% identical to any one of SEQ ID NO: 3, 6, 9, 17, 19, 21, or 23, and/or wherein the polypeptide catalyses the conversion of a substrate in the epoxy-janthitrem biosynthetic pathway, and wherein the nucleic acid sequence encoding the polypeptide is heterologous to the transcription control sequence.

In another aspect, the present invention relates to a vector capable of expressing a polypeptide as described herein, or comprising a polynucleotide encoding a polypeptide as defined herein.

In another aspect, the present invention relates to a host cell comprising a polynucleotide as described herein or a vector as described herein, wherein the polynucleotide is heterologous to the host cell. In various embodiments, the host cell comprises two or more polynucleotides as described herein, or a vector comprising two or more polynucleotides as described herein, or two or more vectors as described herein, wherein one or more of said polynucleotides is heterologous to the host cell.

In various embodiments, the host cell comprises two or more heterologous polypeptides as described herein.

In one embodiment, the one or more heterologous polypeptides is encoded by a nucleic acid sequence heterologous to the host cell.

In one embodiment, one or more of the heterologous polypeptides catalyses the production of an epoxy-janthitrem compound of formula I or formula II.

In one example, each of the heterologous polypeptides is involved in the epoxy-janthitrem biosynthetic pathway. For example, each of the heterologous polypeptides catalyses the production of an epoxy-janthitrem compound of formula I or formula II.

In one embodiment, the host cell comprises an endophytic symbiont.

In one embodiment, the host cell is an Aspergillus cell, such as an Aspergillus albiforma cell, an Aspergillus flavus cell, or an Aspergillus oryzae cell.

In one embodiment, the host cell is an Periglandula cell.

In one embodiment, the host cell is an Epichloë cell.

In one embodiment, in the absence of the heterologous polypeptide, the Epichloë cell is unable to synthesise one or more epoxy-janthitrem compounds and/or is unable to synthesise a compound of formula I.

In one embodiment, the host cell is a cell from an Epichloë selected from the group consisting of Epichloë aotearoae, Epichloë bromicola, Epichloë coenophiala (Epichloë taxonomic group FaTG-3), Epichloë festucae, Epichloë funkii, Epichloë hybrida (Epichloë taxonomic group LpTG-2), Epichloë occultans, Epichloë siegelii, Epichloë taxonomic group LpTG-3, Epichloë taxonomic group FaTG-2, and Epichloë taxonomic group FaTG-4.

In one example, the host cell is a cell from the Epichloë taxonomic group E. festucae var. lolii, also known as Epichloë taxonomic group LpTG-1.

In one embodiment, the host cell is selected from the group consisting of: Epichloë festucae var. lolii AR1, Epichloë festucae var. lolii ARS, Epichloë festucae var. lolii AR6, Epichloë festucae var. lolii AR48, Epichloë festucae var. lolii AR3060, Epichloë festucae var. lolii E2368, Epichloë festucae var. lolii Fg1, and Epichloë festucae var. lolii FI1.

In one embodiment, the host cell comprises one or more functional genes selected from the group comprising idtG, idtM, idtB, idtC, idtP, idtQ, idtF, and idtK.

In one embodiment, the host cell comprises each of the genes from the group comprising idtG, idtM, idtB, idtC, idtP, and idtQ.

In one embodiment, the host cell comprises the gene idtF.

In one embodiment, the host cell comprises the gene idtK.

In one embodiment, the host cell has been modified or transformed to comprise a polynucleotide encoding the gene idtF.

In one embodiment, the host cell has been modified or transformed to comprise a polynucleotide encoding the gene idtK.

In another aspect, the present invention relates to a method for preparing a polypeptide that catalyses the conversion of a substrate in the epoxy-janthitrem biosynthetic pathway, the method comprising the step of culturing a host cell as described herein under conditions that provide for expression of the polypeptide.

In one embodiment, the method further comprises purifying the polypeptide.

In another aspect, the present invention relates to a method of making an epoxy-janthitrem compound, comprising:
a. contacting an indole diterpene precursor with a polypeptide as defined herein to produce an epoxy-janthitrem compound; and
b. optionally, isolating the epoxy-janthitrem compound produced in step (a);

wherein if the epoxy-janthitrem compound is produced in a host cell, the polypeptide is heterologous to the host cell or is heterologously expressed by the host cell.

In one embodiment, the epoxy-janthitrem compound is a compound of formula I or formula II.

In one embodiment, when produced in a host cell, the polypeptide as defined herein is heterologous to the cell and the indole diterpene precursor is present in and/or expressed by the same cell as the polypeptide, and the step of contacting the indole diterpene precursor occurs in the host cell.

In one embodiment, the method further comprises isolating an epoxy-janthitrem compound produced by the host cell.

In one embodiment, the indole diterpene precursor is selected from the group comprising isopentyl pyrophosphate, farnesyl pyrophosphate, and indole-3-glycerol phosphate.

In one embodiment, the indole diterpene precursor is selected from the group comprising terpendole C, terpendole J, terpendole I, terpendole B, terpendole G, terpendole F, terpendole E, α-paxitriol, α-PC-M6, paspaline B, intermediate 1, paspaline, and emindole SB, or any combination thereof.

In another aspect, the present invention relates to a method of providing or modifying production of one or more indole diterpene compounds in a host cell or organism.

In one embodiment, the host cell or organism comprises at least one genetic modification associated with altered regulation or production of one or more gene products encoded by a gene selected from the group consisting of idtA, idtD, and idtO.

In one embodiment, the host cell or organism comprises a functional cytochrome P450 monooxygenase activity, such as a functional cytochrome P450 monooxygenase encoded by the idtQ gene.

In one embodiment, the host cell or organism is a host cell or organism capable of terpendole I production. In one embodiment, the host cell or organism is a host cell or organism capable of producing one or more epoxy-janthitrem compounds.

In one embodiment, the method of modifying production of one or more indole diterpene compounds is a method of decreasing the production or amount of at least one indole diterpene compound. In one example, the method of modifying production of one or more indole diterpene compounds is a method of decreasing the production or amount of at least one indole diterpene compound and increasing the production or amount of at least one other indole diterpene compound.

In various embodiments, at least one of the indole diterpene compounds is a terpendole. In another embodiment, at least one of the indole diterpene compounds is an epoxy-janthitrem compound.

In one embodiment, the method of modifying production of one or more indole diterpene compounds is a method of decreasing the production or amount of at least one terpendole compound. In one example, the method of modifying production of one or more indole diterpene compounds is a method of decreasing the production or amount of at least one terpendole compound and increasing the production or amount of at least one epoxy-janthitrem compound.

In one embodiment, the method of modifying production of one or more indole diterpene compounds is a method of decreasing the production or amount of at least one epoxy-janthitrem compound. In one example, the method of modifying production of one or more indole diterpene compounds is a method of decreasing the production or amount of at least one epoxy-janthitrem compound and increasing the production or amount of at least one terpendole compound.

In one embodiment, the method of modifying production of one or more indole diterpene compounds is a method of increasing the production or amount of at least one indole diterpene compound.

In one embodiment, the method of modifying production of one or more indole diterpene compounds is a method of increasing the production or amount of at least one terpendole compound. In one example, the method of modifying production of one or more indole diterpene compounds is a method of increasing the production or amount of at least one terpendole compound and decreasing the production or amount of at least one epoxy-janthitrem compound.

In one embodiment, the method of modifying production of one or more indole diterpene compounds is a method of increasing the production or amount of at least one epoxy-janthitrem compound. In one example, the method of modifying production of one or more indole diterpene compounds is a method of increasing the production or amount of at least one epoxy-janthitrem compound and decreasing the production or amount of at least one terpendole compound. In another example, the method of modifying production of one or more indole diterpene compounds is a method of increasing the production or amount of at least one epoxy-janthitrem compound and decreasing the production or amount of at least one other epoxy-janthitrem compound.

Specifically contemplated examples of methods of modifying production of one or more indole diterpene compounds are presented herein in the Examples.

In another aspect, the present invention relates to a method of providing or modifying production of one or more indole diterpene compounds in a host cell or organism capable of terpendole I production comprising: introducing into said host cell or organism at least one genetic modification associated with altered regulation or production of one or more gene products encoded by a gene selected from the group consisting of idtA, idtD, idtF, and idtO, and wherein production of one or more epoxy-janthitrem compounds in the host cell or organism is provided or modified.

In another aspect, the present invention relates to a method of providing or modifying production of one or more indole diterpene compounds in a host cell or organism capable of terpendole I production comprising: introducing into said host cell or organism at least one genetic modification associated with altered regulation or production of one or more gene products encoded by a gene selected from the group consisting of idtA, idtD, and idtO, and wherein production of one or more epoxy-janthitrem compounds in the host cell or organism is provided or modified.

In various embodiments of the methods, cells, and organisms contemplated herein, selection and/or expression, including the modulation of expression, of one or more polypeptides as defined herein, optionally in conjunction with modulation of the activity of one or more genes or gene products involved in the indole diterpene biosynthetic pathway, and optionally together with appropriate selection of the host cell or organism including selection with respect to the presence of absence of one or more functional genes involved in the indole diterpene biosynthetic pathway, enables modification of the production of one or more indole diterpene compounds, including the production of one or more desired indole diterpene compounds without the concomitant production of one or more other indole diterpene compounds or with reduced production of one or more other indole diterpene compounds, and/or the production of one or more desired indole diterpene compounds without the concomitant production of one or more less desirable compounds or with reduced production of one or more less desirable compounds.

In one embodiment, the method of providing or modifying production of one or more indole diterpene compounds in a host cell or organism comprises introducing into said host cell or organism at least one genetic modification in an idtA gene that reduces or prevents the production or activity of an idtA gene product, wherein the production or amount of one or more epoxy-janthitrem compounds is reduced when compared to a host cell or organism in which such a genetic modification in an idtA gene is not present.

In one embodiment, the method of providing or modifying production of one or more indole diterpene compounds in a host cell or organism comprises introducing into said host cell or organism at least one genetic modification in an idtA gene that reduces or prevents the production or activity of an idtA gene product, wherein the production or amount of epoxy-janthitrem I is reduced when compared to a host cell or organism in which such a genetic modification in an idtA gene is not present.

In one embodiment, the method of providing or modifying production of one or more indole diterpene compounds in a host cell or organism comprises introducing into said host cell or organism at least one genetic modification in an idtA gene that reduces or prevents the production or activity of an idtA gene product, wherein the production or amount of epoxy-janthitrem IV is reduced when compared to a host cell or organism in which such a genetic modification in an idtA gene is not present.

In one example, the production or amount of both epoxy-janthitrem I and epoxy-janthitrem IV is reduced when compared to a host cell or organism in which such a genetic modification in an idtA gene is not present.

In a further example, the production or amount of both epoxy-janthitrem I and epoxy-janthitrem IV is reduced and the production or amount of one or both of epoxy-janthitriol and epoxy-janthitrem III is not substantially reduced when compared to a host cell or organism in which such a genetic modification in an idtA gene is not present.

In one embodiment, the method of providing or modifying production of one or more indole diterpene compounds in a host cell or organism comprises introducing into said host cell or organism at least one genetic modification in an idtA gene that reduces or prevents the production or activity of an idtA gene product, wherein the production or amount of one or more terpendole compounds is increased when compared to a host cell or organism in which such a genetic modification in an idtA gene is not present.

In one embodiment, the method of providing or modifying production of one or more indole diterpene compounds in a host cell or organism comprises introducing into said host cell or organism at least one genetic modification in an idtA gene that reduces or prevents the production or activity of an idtA gene product, wherein the production or amount of one or more epoxy-janthitrem compounds is reduced and the production or amount of one or more terpendole compounds is increased when compared to a host cell or organism in which such a genetic modification in an idtA gene is not present.

In one embodiment, the method of providing or modifying production of one or more indole diterpene compounds in a host cell or organism comprises introducing into said host cell or organism at least one genetic modification in an idtD gene that reduces or prevents the production or activity of an idtD gene product, wherein the production or amount of one or more epoxy-janthitrem compounds is reduced when compared to a host cell or organism in which such a genetic modification in an idtD gene is not present.

In one example, the production or amount of epoxy-janthitriol is reduced when compared to a host cell or organism in which such a genetic modification in an idtD gene is not present.

In one example, the production or amount of epoxy-janthitrem I is reduced when compared to a host cell or organism in which such a genetic modification in an idtD gene is not present.

In one example, the production or amount of epoxy-janthitrem II is reduced when compared to a host cell or organism in which such a genetic modification in an idtD gene is not present.

In one example, the production or amount of epoxy-janthitrem III is reduced when compared to a host cell or organism in which such a genetic modification in an idtD gene is not present.

In one example, the production or amount of epoxy-janthitrem IV is reduced when compared to a host cell or organism in which such a genetic modification in an idtD gene is not present.

In one example, the production or amount of each of epoxy-janthitrem I, epoxy-janthitrem II, epoxy-janthitrem III, and epoxy-janthitrem IV is reduced when compared to a host cell or organism in which such a genetic modification in an idtD gene is not present.

In one example, the production or amount of each of epoxy-janthitriol, epoxy-janthitrem I, epoxy-janthitrem II, epoxy-janthitrem III, and epoxy-janthitrem IV is reduced when compared to a host cell or organism in which such a genetic modification in an idtD gene is not present.

In one embodiment, the method of providing or modifying production of one or more indole diterpene compounds in a host cell or organism comprises introducing into said host cell or organism at least one genetic modification in an idtD gene that reduces or prevents the production or activity of an idtD gene product, wherein the production or amount of one or more terpendole compounds is increased when compared to a host cell or organism in which such a genetic modification in an idtD gene is not present.

In one embodiment, the method of providing or modifying production of one or more indole diterpene compounds in a host cell or organism comprises introducing into said host cell or organism at least one genetic modification in an idtO gene that reduces or prevents the production or activity of an idtO gene product, wherein the production or amount of one or more epoxy-janthitrem compounds is reduced when compared to a host cell or organism in which such a genetic modification in an idtO gene is not present.

In one example, the production or amount of epoxy-janthitriol is reduced when compared to a host cell or organism in which such a genetic modification in an idtO gene is not present.

In one example, the production or amount of epoxy-janthitrem I is reduced when compared to a host cell or organism in which such a genetic modification in an idtO gene is not present.

In one example, the production or amount of epoxy-janthitrem II is reduced when compared to a host cell or organism in which such a genetic modification in an idtO gene is not present.

In one example, the production or amount of epoxy-janthitrem III is reduced when compared to a host cell or organism in which such a genetic modification in an idtO gene is not present.

In one example, the production or amount of epoxy-janthitrem IV is reduced when compared to a host cell or organism in which such a genetic modification in an idtO gene is not present.

In one example, the production or amount of each of epoxy-janthitrem I, epoxy-janthitrem II, epoxy-janthitrem III, and epoxy-janthitrem IV is reduced when compared to a host cell or organism in which such a genetic modification in an idtO gene is not present.

In one example, the production or amount of each of epoxy-janthitriol, epoxy-janthitrem I, epoxy-janthitrem II, epoxy-janthitrem III, and epoxy-janthitrem IV is reduced when compared to a host cell or organism in which such a genetic modification in an idtO gene is not present.

In one embodiment, the method of providing or modifying production of one or more indole diterpene compounds in a host cell or organism comprises introducing into said host cell or organism at least one genetic modification in an idtO gene that reduces or prevents the production or activity of an idtO gene product, wherein the production or amount of one or more terpendole compounds is increased when compared to a host cell or organism in which such a genetic modification in an idtO gene is not present.

In one embodiment, the method of providing or modifying production of one or more indole diterpene compounds in a host cell or organism comprises introducing into said host cell or organism at least one genetic modification in an idtF gene that reduces or prevents the production or activity of an idtF gene product, wherein the production or amount of one or more epoxy-janthitrem compounds is reduced when compared to a host cell or organism in which such a genetic modification in an idtF gene is not present.

In one example, the production or amount of epoxy-janthitrem II is reduced when compared to a host cell or organism in which such a genetic modification in an idtF gene is not present.

In one example, the production or amount of epoxy-janthitrem III is reduced when compared to a host cell or organism in which such a genetic modification in an idtF gene is not present.

In one example, the production or amount of epoxy-janthitrem IV is reduced when compared to a host cell or organism in which such a genetic modification in an idtF gene is not present.

In one example, the production or amount of each of epoxy-janthitrem II, epoxy-janthitrem III, and epoxy-janthitrem IV is reduced when compared to a host cell or organism in which such a genetic modification in an idtF gene is not present.

In one example, the production or amount of epoxy-janthitriol or of epoxy-janthitrem I or of both epoxy-janthitriol and epoxy-janthitrem I is not substantially reduced when compared to a host cell or organism in which such a genetic modification in an idtF gene is not present.

In a further example, the production or amount of one or more of epoxy-janthitrem II, epoxy-janthitrem III, and epoxy-janthitrem IV, is reduced and the production or amount of one or both of epoxy-janthitriol and epoxy-janthitrem I is not substantially reduced when compared to a host cell or organism in which such a genetic modification in an idtF gene is not present.

In one embodiment, the method of providing or modifying production of one or more indole diterpene compounds in a host cell or organism comprises introducing into said host cell or organism at least one genetic modification in an idtF gene that reduces or prevents the production or activity of an idtF gene product, wherein the production or amount of one or more terpendole compounds is increased when compared to a host cell or organism in which such a genetic modification in an idtF gene is not present.

In one embodiment, the method of providing or modifying production of one or more indole diterpene compounds in a host cell or organism comprises introducing into said host cell or organism at least one genetic modification in an idtA gene that reduces or prevents the production or activity of an idtA gene product and at least one genetic modification in an idtF gene that reduces or prevents the production or activity of an idtF gene product. In one example, the production or amount of one or more epoxy-janthitrem compounds is reduced when compared to a host cell or organism in which the at least one genetic modification in an idtA gene is not present. In another example, the production or amount of one or more epoxy-janthitrem compounds is reduced when compared to a host cell or organism in which the at least one genetic modification in an idtF gene is not present. In a further example, the production or amount of one or more epoxy-janthitrem compounds is reduced when compared to a host cell or organism in which the at least one genetic modification in both an idtA gene and an idtF gene is not present.

In one embodiment, the method of providing or modifying production of one or more indole diterpene compounds in a host cell or organism comprises introducing into said host cell or organism at least one genetic modification in an idtA gene that reduces or prevents the production or activity of an idtA gene product and at least one genetic modification in an idtF gene that reduces or prevents the production or activity of an idtF gene product, wherein the prodfuction or amount of epoxy-janthitrem I is reduced when compared to a host cell or organism in which the at least one genetic modification in an idtA gene is not present.

In one embodiment, the method of providing or modifying production of one or more indole diterpene compounds in a host cell or organism comprises introducing into said host cell or organism at least one genetic modification in an idtA gene that reduces or prevents the production or activity of an idtA gene product and at least one genetic modification in an idtF gene that reduces or prevents the production or activity of an idtF gene product, wherein the production or amount of epoxy-janthitrem IV is reduced when compared to a host cell or organism in which the at least one genetic modification in an idtA gene and/or in an idtF gene is not present.

In one example, the production or amount of epoxy-janthitrem II is reduced when compared to a host cell or organism in which the at least one genetic modification in an idtA gene and/or in an idtF gene is not present.

In one example, the production or amount of epoxy-janthitrem III is reduced when compared to a host cell or organism in which the at least one genetic modification in an idtA gene and/or in an idtF gene is not present.

In one example, the production or amount of epoxy-janthitrem IV is reduced when compared to a host cell or organism in which the at least one genetic modification in an idtA gene and/or in an idtF gene is not present.

In one example, the production or amount of each of epoxy-janthitrem I, epoxy-janthitrem II, epoxy-janthitrem III, and epoxy-janthitrem IV is reduced when compared to a host cell or organism in which the at least one genetic modification in an idtA gene and/or in an idtF gene is not present.

In a further example, the production or amount of each of epoxy-janthitrem II, epoxy-janthitrem III, and epoxy-janthitrem IV is reduced and the production or amount of epoxy-janthitriol is not substantially reduced when compared to a host cell or organism in which the at least one genetic modification in an idtA gene and/or in an idtF gene is not present.

In one embodiment, the method of providing or modifying production of one or more indole diterpene compounds in a host cell or organism comprises introducing into said host cell or organism at least one genetic modification in an idtA gene that reduces or prevents the production or activity of an idtA gene product and at least one genetic modification in an idtF gene that reduces or prevents the production or activity of an idtF gene product, wherein the production or amount of one or more terpendole compounds is increased when compared a host cell or organism in which the at least one genetic modification in an idtA gene and/or in an idtF gene is not present.

In one embodiment, the method of providing or modifying production of one or more indole diterpene compounds in a host cell or organism comprises introducing into said host cell or organism at least one genetic modification in an idtA gene that reduces or prevents the production or activity of an idtA gene product and at least one genetic modification in an idtF gene that reduces or prevents the production or activity of an idtF gene product, wherein the production or amount of one or more epoxy-janthitrem compounds is reduced and the production or amount of one or more terpendole compounds is increased when compared a host cell or organism in which the at least one genetic modification in an idtA gene and/or in an idtF gene is not present.

In one example, the terpendole compound is terpendole I.

In various embodiments, the genetic modification is introduced by gene editing, such as by CRISPR/Cas editing. In one example, the genetic modification is introduced by gene editing as exemplified herein, for example using any one or more oligonucleotides, polynucleotides, constructs or vectors as described herein in the Examples or as presented herein in any one of SEQ ID NOs: 24 to 49 or 54 to 69.

In one embodiment, the method of providing or modifying production of one or more indole diterpene compounds in a host cell or organism comprises introducing into said host cell or organism at least one genetic modification in an idtD gene that reduces or prevents the production or activity of an idtD gene product, wherein the production or amount of one or more epoxy-janthitrem compounds is reduced and the production or amount of one or more terpendole compounds is increased when compared a host cell or organism in which such a genetic modification in an idtD gene is not present.

In another aspect, the present invention relates to a method of providing or modifying production of one or more indole diterpene compounds in a host cell or organism capable of terpendole I production comprising: introducing into said host cell or organism at least one genetic modification associated with altered regulation or production of one or more gene products encoded by a gene selected from the group consisting of idtA, idtD, idtF, and idtO, wherein the host cell or organism comprises one or more heterologous polypeptides as herein described, and wherein production of one or more epoxy-janthitrem compounds in the host cell or organism is provided or modified.

In various embodiments, the one or more indole diterpene compounds is one or more terpendole compounds.

In various embodiments, the one or more indole diterpene compounds is one or more epoxy-janthitrem compounds. In one example, the one or more indole diterpene compounds is one or more epoxy-janthitrem compounds of formula I or formula II.

In various embodiments, the one or more heterologous polypeptides is heterologously expressed by the host cell. In various embodiments, one or more of the one or more polypeptides comprises an amino acid sequence at least about 70%, at least 75%, at least 80%, at least 85%, at least 90%, or at least about 95% identical to any one of SEQ ID NO: 3, 6, 9, 17, 19, 21, 23, 50-53, or 70-74, and/or the polypeptide catalyses the conversion of a substrate in the epoxy-janthitrem biosynthetic pathway.

In one embodiment, the host cell comprises a vector capable of expressing a polypeptide as described herein, or comprising a polynucleotide encoding a polypeptide as defined herein.

In one embodiment, the one or more heterologous polynucleotides or the one or more polynucleotides encoding one or more heterologous polypeptides are introduced into the host cell by a method as herein described or exemplified, for example by a gene editing method.

In another aspect, the present invention relates to a host cell or organism capable of terpendole I production into which at least one genetic modification associated with altered regulation or production of one or more gene products encoded by a gene selected from the group consisting of idtA, idtD, idtF, and idtO has been introduced, and wherein the host cell comprises one or more heterologous polynucleotides as described herein, one or more vectors as described herein, or one or more heterologous polypeptides as described herein.

In various embodiments, the host cell comprises two or more heterologous polypeptides as described herein.

In one embodiment, one or more of the heterologous polypeptides catalyses the production of an epoxy-janthitrem compound of formula I or formula II.

In one example, each of the heterologous polypeptides is involved in the epoxy-janthitrem biosynthetic pathway. For example, each of the heterologous polypeptides catalyses the production of an epoxy-janthitrem compound of formula I or formula II.

In another aspect, the present invention relates to a method of providing or modifying production of one or more epoxy-janthitrem compounds in a host cell or organism comprising a functional cytochrome P450 monooxygenase activity, such as a functional cytochrome P450 monooxygenase encoded by the idtQ gene, the method comprising: expressing a polypeptide as defined herein in the host cell or organism under conditions effective to produce one or more epoxy-janthitrem compounds, wherein said polypeptide is heterologous to the host cell or organism, and wherein the polypeptide as defined herein replaces an inactive or deleted activity, introduces a new activity, or enhances an existing activity in the host cell or organism, and wherein production of one or more epoxy-janthitrem compounds in the host cell or organism is provided or modified.

In another aspect, the present invention relates to a method of providing or modifying production of one or more epoxy-janthitrem compounds in a host cell or organism capable of terpendole I production comprising: expressing one or more polypeptides as defined herein in the organism under conditions effective to produce one or more epoxy-janthitrem compounds, wherein said one or more polypeptides is heterologous to the host cell or organism, and wherein the one or more polypeptides as defined herein replaces an inactive or deleted activity, introduces a new activity, or enhances an existing activity in the host cell or organism, and wherein production of one or more epoxy-janthitrem compounds in the host cell or organism is provided or modified.

In another aspect, the present invention relates to a method of providing or increasing production of one or more epoxy-janthitrem compounds in a host cell or organism comprising a functional cytochrome P450 monooxygenase activity, such as a functional cytochrome P450 monooxygenase encoded by the idtQ gene, the method comprising: expressing a polypeptide as defined herein in the host cell or organism under conditions effective to produce one or more epoxy-janthitrem compounds, wherein said polypeptide is heterologous to the host cell or organism, and wherein the polypeptide as defined herein replaces an inactive or deleted activity, introduces a new activity, or enhances an existing activity in the host cell or organism, and wherein production of one or more epoxy-janthitrem compounds in the host cell or organism is provided or increased.

In another aspect, the present invention relates to a genetically modified host cell capable of producing one or more indole diterpene compounds, wherein the host cell comprises at least one genetic modification associated with altered regulation or production of one or more gene products encoded by a gene selected from the group consisting of idtA, idtD, idtO, idtG, idtM, idtB, idtC, idtP, idtQ, idtF, and idtK.

In another aspect, the present invention relates to a genetically modified host cell capable of producing one or more indole diterpene compounds, wherein the host cell comprises at least one genetic modification associated with altered regulation or production of one or more gene products encoded by a gene selected from the group consisting of idtA, idtD, and idtO.

In one embodiment, the gene product catalyses the production of an epoxy-janthitrem compound of formula I or formula II.

In one embodiment, the host cell comprises an endophytic symbiont.

In one embodiment, the host cell is an Epichloë cell.

In one embodiment, in the absence of the genetic modification, the Epichloë cell or a symbiont comprising same is able to synthesise one or more epoxy-janthitrem compounds and/or is able to synthesise a compound of formula I.

In one embodiment, the host cell into which the one or more genetic modifications is introduced is selected from the group consisting of: Epichloë festucae var. lolii AR37, Epichloë festucae var. lolii AR40, Epichloë festucae var. lolii AR127, Epichloë festucae var. lolii AR128, adnd Epichloë festucae var. lolii AR166.

In one embodiment, the host cell comprises one or more genes selected from the group consisting of idtA, idtD, and idtO.

In one embodiment, the host cell into which the one or more genetic modifications is or has been introduced is selected from the group consisting of: Epichloë festucae var. lolii AR1, Epichloë festucae var. lolii ARS, Epichloë festucae var. lolii AR6, Epichloë festucae var. lolii AR48, Epichloë festucae var. lolii AR3060, Epichloë festucae var. lolii E2368, Epichloë festucae var. lolii Fg1, and Epichloë festucae var. lolii FI1.

In one embodiment, the host cell comprises one or more functional genes selected from the group comprising idtG, idtM, idtB, idtC, idtP, idtQ, idtF, and idtK.

In one embodiment, the host cell comprises each of the genes from the group comprising idtG, idtM, idtB, idtC, idtP, and idtQ.

In one embodiment, the host cell comprises the gene idtF.

In one embodiment, the host cell comprises the gene idtK.

In one embodiment, the host cell has been modified or transformed to comprise a polynucleotide encoding the gene idtF.

In one embodiment, the at least one genetic modification is associated with altered regulation or production of one or more gene products encoded by the idtA gene. In one example, at least one genetic modification reduces or prevents production of a functional IdtA polypeptide, for example, reduces or prevents production of a catalytically functional IdtA polypeptide.

In one embodiment, the at least one genetic modification is a modification that alters the amino acid sequence of the IdtA polypeptide, such as a nucleotide insertion, deletion or substitution in the idtA gene. In one embodiment, the at least one genetic modification results in a truncated idtA gene product, such as a truncated IdtA polypeptide. In one example, the modification alters the amino acid sequence of the IdtA polypeptide as depicted in FIG. 19 or FIG. 20, and/or results in an idtA gene product encoding or having a predicted amino acid sequence as presented in SEQ ID NO: 52 or SEQ ID NO: 53.

In one embodiment, the at least one genetic modification is associated with altered regulation or production of one or more gene products encoded by the idtD gene. In one example, at least one genetic modification reduces or prevents production of a functional IdtD polypeptide, for example, reduces or prevents production of a catalytically functional IdtD polypeptide.

In one embodiment, the at least one genetic modification is a modification that alters the amino acid sequence of the IdtD polypeptide, such as a nucleotide insertion, deletion or substitution in the idtD gene. In one embodiment, the at least one genetic modification results in a truncated idtD gene product, such as a truncated IdtD polypeptide. In one example, the modification alters the amino acid sequence of the IdtD polypeptide as depicted in FIG. 16 or FIG. 17, and/or results in an idtD gene product encoding or having a predicted amino acid sequence as presented in SEQ ID NO: 50 or SEQ ID NO: 51.

In one embodiment, the at least one genetic modification is associated with altered regulation or production of one or more gene products encoded by the idtO gene. In one example, at least one genetic modification reduces or prevents production of a functional IdtO polypeptide, for example, reduces or prevents production of a catalytically functional IdtO polypeptide.

In one embodiment, the at least one genetic modification is a modification that alters the amino acid sequence of the IdtO polypeptide, such as a nucleotide insertion, deletion or substitution in the idtO gene. In one embodiment, the at least one genetic modification results in a truncated idtO gene product, such as a truncated IdtO polypeptide. In one example, the modification alters the amino acid sequence of the IdtO polypeptide as depicted in FIG. 22 or FIG. 23, and/or results in an idtO gene product encoding or having a predicted amino acid sequence as presented in SEQ ID NO: 70 or SEQ ID NO: 71.

In one embodiment, the at least one genetic modification is associated with altered regulation or production of one or more gene products encoded by the idtF gene. In one example, at least one genetic modification reduces or prevents production of a functional IdtF polypeptide, for example, reduces or prevents production of a catalytically functional IdtF polypeptide.

In one embodiment, the at least one genetic modification is a modification that alters the amino acid sequence of the IdtF polypeptide, such as a nucleotide insertion, deletion or substitution in the idtOFgene. In one embodiment, the at least one genetic modification results in a truncated idtF gene product, such as a truncated IdtF polypeptide. In one example, the modification alters the amino acid sequence of the IdtF polypeptide as depicted in any one of FIG. 25, 26 or 27, and/or results in an idtF gene product encoding or having a predicted amino acid sequence as presented in SEQ ID NO: 72, 73, or 74.

In another aspect, the present invention relates to a method of providing or increasing production of one or more epoxy-janthitrem compounds in a host cell or organism capable of terpendole I production comprising: expressing one or more polypeptides as defined herein in the organism under conditions effective to produce one or more epoxy-janthitrem compounds, wherein said one or more polypeptides is heterologous to the host cell or organism, and wherein the one or more polypeptides as defined herein replaces an inactive or deleted activity, introduces a new activity, or enhances an existing activity in the host cell or organism, and wherein production of one or more epoxy-janthitrem compounds in the host cell or organism is provided or increased.

In various embodiments of the methods, cells, and organisms contemplated herein, selection and/or expression, including the modulation of expression, of one or more polypeptides as defined herein, optionally in conjunction with modulation of the activity of one or more genes or gene products involved in the indole diterpene biosynthetic pathway, and optionally together with appropriate selection of the host cell or organism including selection with respect to the presence of absence of one or more functional genes involved in the indole diterpene biosynthetic pathway, enables the production of one or more epoxy-janthitrem compounds, including the production of one or more desired epoxy-janthitrem compounds without the concomitant production of one or more other epoxy-janthitrem compounds or with reduced production of one or more other epoxy-janthitrem compounds, and/or the production of one or more desired epoxy-janthitrem compounds without the concomitant production of one or more less desirable compounds, or with reduced production of one or more less desirable compounds.

In one embodiment, the organism comprises one or more functional genes selected from the group comprising idtG, idtM, idtB, idtC, idtP, idtQ, idtF, and idtK.

In one embodiment, the organism comprises each of the genes from the group comprising idtG, idtM, idtB, idtC, idtP, and idtQ.

In one embodiment, the organism comprises the gene idtF.

In one embodiment, the organism comprises the gene idtK.

In one embodiment, the epoxy-janthitrem compound is a compound of formula I or formula II.

In one embodiment, the epoxy-janthitrem compound is selected from the group consisting of epoxy-janthitriol, epoxy-janthitrem I, epoxy-janthitrem II, epoxy-janthitrem III, and epoxy-janthitrem IV.

In one embodiment, the epoxy-janthitrem compound is selected from the group consisting of epoxy-janthitrem I, epoxy-janthitrem II, epoxy-janthitrem III, and epoxy-janthitrem IV.

In one embodiment, a single epoxy-janthitrem compound is produced. In one example, only epoxy-janthitriol is produced. In another example, only epoxy-janthitrem I is produced. In another example, only epoxy-janthitrem II is produced. In still another example, only epoxy-janthitrem III is produced. In a further example, only epoxy-janthitrem IV is produced.

In various embodiments, a mixture of epoxy-janthitrem compounds is produced. In one example, epoxy-janthitriol, epoxy-janthitrem II and epoxy-janthitrem III are produced. In one example, epoxy-janthitrem II and epoxy-janthitrem III are produced. In one example, epoxy-janthitriol, epoxy-janthitrem I, epoxy-janthitrem III, and epoxy-janthitrem IV are produced. In one example, epoxy-janthitrem I, epoxy-janthitrem III, and epoxy-janthitrem IV are produced.

In a further example, epoxy-janthitriol, epoxy-janthitrem II, epoxy-janthitrem III, and epoxy-janthitrem IV are produced. In another example, epoxy-janthitrem II, epoxy-janthitrem III, and epoxy-janthitrem IV are produced.

In one embodiment, epoxy-janthitrem I is not substantially produced.

In one embodiment, epoxy-janthitrem II is not substantially produced.

In one embodiment, epoxy-janthitrem III is not substantially produced.

In one embodiment, epoxy-janthitrem IV is not substantially produced.

In one embodiment, epoxy-janthitriol is not substantially produced.

In one embodiment, epoxy-janthitrem I and epoxy-janthitrem II are not substantially produced. In one embodiment, each of epoxy-janthitrem I, epoxy-janthitrem II, and epoxy-janthitriol are not substantially produced.

In one embodiment, epoxy-janthitrem I and epoxy-janthitrem IV are not substantially produced.

In one embodiment, epoxy-janthitrem III and epoxy-janthitrem IV are not substantially produced. In one embodiment, each of epoxy-janthitrem III, epoxy-janthitrem IV, and epoxy-janthitriol are not substantially produced.

In one embodiment, one or more of epoxy-janthitrem I, epoxy-janthitrem II, and epoxy-janthitrem IV are not substantially produced. For example, each of epoxy-janthitrem I, epoxy-janthitrem II, and epoxy-janthitrem IV are not substantially produced.

In one embodiment, one or more of epoxy-janthitrem II, epoxy-janthitrem III, and epoxy-janthitrem IV are not substantially produced. For example, each of epoxy-janthitrem II, epoxy-janthitrem III, and epoxy-janthitrem IV are not substantially produced.

In another aspect, the present invention relates to a genetically modified host cell comprising a polypeptide comprising an amino acid sequence having at least 70%, at least 75%, at least 80%, at least 85%, or at least about 90% identity with an amino acid sequence set forth in any one of SEQ ID NO: 3, 6, 9, 17, 19, 21, 23, 50-53, or 70-74, wherein the polypeptide is heterologous to the host cell.

In another aspect, the present invention relates to a genetically modified host cell comprising a polypeptide comprising an amino acid sequence having at least 70%, at least 75%, at least 80%, at least 85%, or at least about 90% identity with an amino acid sequence set forth in any one of SEQ ID NO: 3, 6, 9, 17, 19, 21, or 23, wherein the polypeptide is heterologous to the host cell, and wherein the polypeptide catalyzes one of the following reactions:

a. the conversion of terpendole Ito epoxy-janthitriol;

b. the conversion of terpendole J to epoxy-janthitrem III;

c. the conversion of terpendole C to epoxy-janthitrem II;

d. the conversion of epoxy-janthitriol to epoxy-janthitrem I;

e. the conversion of epoxy-janthitrem III to epoxy-janthitrem IV; or

f. any combination of two or more of a) to e) above.

In one embodiment, the host cell is capable of synthesizing a compound of any one of formulae IV to VIII or uptaking a compound of any one of formulae IV to VIII from its surroundings.

In one embodiment, the host cell further comprises one or more enzymes of a pathway for synthesizing a compound of any one of formulae IV to VIII from a carbon source.

In one embodiment, the pathway for synthesizing a compound of any one of formulae IV to VIII from a carbon source is native to the host cell.

In one embodiment, the pathway for synthesizing a compound of any one of formulae IV to VIII from a carbon source is heterologous to the host cell.

In one embodiment, the polypeptide comprises, consists essentially of, or consists of an amino acid sequence having at least 95% identity with an amino acid sequence set forth in any one of SEQ ID NO: 3, 6, 9, 17, 19, 21, or 23.

In one embodiment, the polypeptide comprises, consists essentially of, or consists of an amino acid sequence having at least 99% identity with the amino acid sequence set forth in any one of SEQ ID NO: 3, 6, 9, 17, 19, 21, or 23.

In one embodiment, the polypeptide comprises, consists essentially of, or consists of an amino acid sequence having at least 95% identity with an amino acid sequence set forth in any one of SEQ ID NO: 3, 6, 9, 17, 19, 21, 23, 50-53, or 70-74.

In one embodiment, the polypeptide comprises, consists essentially of, or consists of an amino acid sequence having at least 99% identity with the amino acid sequence set forth in any one of SEQ ID NO: 3, 6, 9, 17, 19, 21, 23, 50-53, or 70-74.

In one embodiment, the host cell is an Epichloë cell.

In another aspect, the present invention relates to a method of producing a compound of formula I or formula II in a genetically modified host cell, comprising:

a. providing the genetically modified host cell as described herein; and

b. culturing the genetically modified host cell in a medium under a suitable condition; or

c. maintaining the genetically modified host cell as described herein under a suitable condition;

wherein the culturing or the maintaining results in the genetically modified host cell producing a compound of formula I or formula II.

In one embodiment the method further comprises separating a compound of formula I or formula II from the host cell and/or the medium, wherein the separating step is subsequent, concurrent or partially concurrent with the culturing or maintaining step.

In one embodiment, the maintaining is in the presence of one or more cells other than the genetically modified host cell.

In one embodiment, the genetically modified host cell is maintained together with one or more plant or animal cells.

In another aspect, the present invention relates to an Epichloë cell, wherein the Epichloë cell:

a. has been modified or transformed with one or more polynucleotides encoding a polypeptide as defined herein; or

b. is capable of heterologously expressing one or more polypeptides as defined herein; or

c. comprises a polynucleotide encoding a polypeptide as defined herein; or

d. comprises a polynucleotide comprising at least about 90% nucleic acid sequence identity to the nucleic acid sequence set forth in any one of SEQ ID NO: 1, 2, 4, 5, 7, 8, 10 to 16, 18, 20, or 22; or

e. is genetically modified to confer upon the Epichloë cell the capacity to synthesize one or more epoxy-janthitrem compounds, wherein the Epichloë cell did not have such capacity in the absence of the genetic modification; or

f. any combination of two of more of a) to e) above.

In another aspect, the present invention relates to an Epichloë cell, wherein the Epichloë cell:

a. has been modified or transformed with one or more polynucleotides encoding a polypeptide as defined herein; or

b. is capable of heterologously expressing one or more polypeptides as defined herein; or

c. comprises a polynucleotide encoding a polypeptide as defined herein; or

d. comprises a polynucleotide comprising at least about 90% nucleic acid sequence identity to the nucleic acid sequence set forth in any one of SEQ ID NO: 1, 2, 4, 5, 7, 8, 10 to 16, 18, 20, or 22; or

e. is genetically modified to confer upon the Epichloë cell the capacity to synthesize one or more epoxy-janthitrem compounds, wherein the Epichloë cell did not have such capacity in the absence of the genetic modification; or

f. comprises at least one genetic modification associated with altered regulation or production of one or more gene products encoded by a gene selected from the group consisting of idtA, idtD, idt0, idtG, idtM, idtB, idtC, idtP, idtQ, idtF, and idtK; or

g. comprises at least one genetic modification associated with altered regulation or production of one or more gene products encoded by a gene selected from the group consisting of idtA, idtD, and idtO; or

h. comprises a genetic modification introduced by gene editing using any one or more of the oligonucleotides, polynucleotides, constructs or vectors as described herein in the

Examples or as presented herein in any one of SEQ ID NOs: 24 to 49; or

i. any combination of two of more of a) to h) above.

In one embodiment, the one or more epoxy-janthitrem compounds is a compound of formula I or formula II.

In another aspect, the present invention relates to a population of cells comprising one or more plant cells and one or more Epichloë cells as as described herein.

In one embodiment, the one or more plant cells comprise, consist essentially of, or consist of one or more cells from the group consisting of:

a. a Pooideae grass;

b. a perennial ryegrass;

c. an annual ryegrass;

d. a hybrid ryegrass;

e. the genus Lolium;

f. the species Lolium perenne, Lolium multiflorum, and Lolium x hybridum;

g. the genus Festuca;

h. the species Festuca amethystina, Festuca arundinacea, Festuca cinerea, Festuca elegans, Festuca glauca, Festuca idahoensis, Festuca ovine, Festuca pallens, Festuca pratensis, Festuca rubra, Festuca rubra subsp. commutate, Festuca saximontana, and Festuca trachyphylla;

i. the genus Secale;

j. the species Secale cereale.

In various examples, the Lolium is Lolium perenne cv. Samson or Lolium perenne cv. Nui.

In various examples, the Festuca is Festuca arundinaceae cv. Hummer.

In various examples, the Secale is Secale cereale cv. Rahu.

In one embodiment, said population comprise a plant or part thereof.

In another aspect, the present invention relates to a method for conferring on an organism the ability to produce one or more epoxy-janthitrem compounds, the method comprising providing the organism with a host cell modified or transformed with or to comprise one or more polynucleotides encoding a polypeptide as defined herein, or one or more genes involved in the epoxy-janthitrem biosynthetic pathway.

Any of the embodiments described herein can relate to any of the aspects presented herein.

In one embodiment, the host cell does not produce detectable levels of toxins from the lolitrem group or ergovaline group. In one embodiment, the host cell does not produce the toxic alkaloid lolitrem B, or does not produce lolitrem B at a level in excess of 2 ppm. In another embodiment, the host cell does not produce the toxic alkaloid ergovaline, or does not produce the toxic alkaloid ergovaline at a level in excess of 0.5 ppm. For example, both the lolitrem B level and the ergovaline level are below detection levels of less than 0.1 ppm of dry matter.

In another aspect, the invention relates to a composition comprising one or more epoxy-janthitrem compounds produced in accordance with the description herein, for example, one or more epoxy-janthitrem compounds produced by or using a host cell, expression construct, polynucleotide, or polypeptide as herein described, optionally together with one or more carriers, including one or more physiologically acceptable carriers, or one or more agriculturally acceptable carriers. In various embodiments, the compostion comprises one or more epoxy-janthitrem compounds, but is substantially free of any one or more of the group consisting of epoxy-janthitrem I, epoxy-janthitrem II, epoxy-janthitrem III, epoxy-janthitrem IV, and epoxy-janthitriol. For example, the composition is produced by or using a host cell as herein described, wherein the host cell is one in which a single epoxy-janthitrem compound is produced, or one in which only two, only three, or only four of the epoxy-janthitrem compounds comprising the group consisting of epoxy-janthitrem I, epoxy-janthitrem II, epoxy-janthitrem III, epoxy-janthitrem IV, and epoxy-janthitriol, is produced. For example, the composition is produced by or using a host cell as herein described, wherein the host cell is one in which one or more, two or more, three or more, or four of the epoxy-janthitrem compounds comprising the group consisting of epoxy-janthitrem I, epoxy-janthitrem II, epoxy-janthitrem III, epoxy-janthitrem IV, and epoxy-janthitriol, is not produced.

In a further aspect, the invention relates to a method of conferring to a plant a benefit, the method comprising contacting the plant with one or more epoxy-janthitrem compounds produced in accordance with the description herein, for example, one or more epoxy-janthitrem compounds produced by or using a host cell, expression construct, polynucleotide, or polypeptide as herein described, wherein the host cell provides one or more epoxy-janthitrem compounds, or wherein a symbiont comprising a host cell provices one or more epoxy-janthitrem compounds, or the one or more epoxy-janthitrem compounds are present at a level sufficient to confer a benefit to the plant.

In one embodiment, the benefit is protection from stress, such as biotic stress, for example that caused by a pest such as an insect pest, or abiotic stress, such as that caused by water deficit, elevated salt levels, heat, nutrient deficiency, or the like, or both abiotic and biotic stress.

Accordingly, in one embodiment the invention relates to a method of protecting a plant, such as a grass, from stress, comprising contacting the plant with one or more epoxy-janthitrem compounds produced in accordance with the description herein, for example, one or more epoxy-janthitrem compounds produced by or using a host cell, expression construct, polynucleotide, or polypeptide as herein described wherein the host cell provides one or more epoxy-janthitrem compounds, or wherein a symbiont comprising a host cell provices one or more epoxy-janthitrem compounds, or the one or more epoxy-janthitrem compounds are present at a level sufficient to confer protection to the plant.

In one example, the biotic stress is caused by a pest selected from the group consisting of: a root aphid (Aploneura lentisci); pasture mealybug (Balanococcus poae); African black beetle (Heteronychus arator); and porina (Wiseana cervinata and W. copularis); and any combination of two or more thereof.

The invention accordingly further relates to methods for the control of pests, particularly plant pests, including insects or nematodes.

For example, the invention also relates to methods of controlling a pest population. The methods generally involve contacting the pests or the pest population with one or more epoxy-janthitrem compounds produced in accordance with the description herein, for example, one or more epoxy-janthitrem compounds produced by or using a host cell, expression construct, polynucleotide, or polypeptide as herein described. Such methods may be used to kill or reduce the numbers of target pests in a given area, or may be prophylactically applied to a locus, such as an environmental area, to prevent infestation by a susceptible pest.

In one embodiment, the method is a method for controlling one or more insect pests, the method comprising the step of applying to a plant or its surroundings or a locus at which insect pests are present one or more epoxy-janthitrem compounds produced in accordance with the description herein, for example, one or more epoxy-janthitrem compounds produced by or using a host cell, expression construct, polynucleotide, or polypeptide as herein described. In another embodiment, the method comprising applying to a plant or its surroundings or a locus at which insect pests are present a composition as described herein.

In a further aspect the present invention relates to a method of treating or protecting a plant or its surroundings, and/or plant derived materials, from pest infestation wherein the method comprises contacting the plant or its environment with one or more epoxy-janthitrem compounds produced in accordance with the description herein, for example, one or more epoxy-janthitrem compounds produced by or using a host cell, expression construct, polynucleotide, or polypeptide as herein described.

In a further aspect, the invention relates to a method of treating or protecting a plant or its surroundings, and/or plant derived materials, from pest infestation wherein the method comprises contacting the plant or its environment with a host cell, expression construct, polynucleotide, or polypeptide as herein described.

According to a further aspect the present invention relates to a method of controlling and/or preventing a pest infestation characterised by the step of contacting the plant or its environment with a host cell, expression construct, polynucleotide, or polypeptide as herein described.

In various embodiments, the host cell is an endophytic fungi, such as an endophytic fungi that has been modified or transformed with one or more polynucleotides as described herein, such as one or more genes involved in the epoxy-janthitrem biosynthetic pathway, or one or more expression constructs comprising same, or wherein the fungi is capable of heterologously expressing one or more polypeptides as described herein.

The invention further relates to the use of a composition of the invention for the control of one or more pests, including one or more insect or nematode pests.

The use of a composition produced by a method of the invention in the manufacture of a formulation for the control of one or more pests is similarly contemplated.

In a further aspect, the invention relates to a plant comprising one or more host cells, wherein the one or more host cells has been modified or transformed with one or more polynucleotides as described herein, such as one or more genes involved in the epoxy-janthitrem biosynthetic pathway, or one or more expression constructs comprising same, or wherein the one or more host cells is capable of heterologously expressing one or more polypeptides as described herein.

In agricultural and horticultural applications, the invention is applicable to any plant or its surroundings. Particularly contemplated plants are monocotyledonous plants of the order Poales, including grasses of the family Poaceae.

In one embodiment, the plant is a Pooideae grass.

In one embodiment, the plant is a perennial, annual or hybrid ryegrass.

In one embodiment, the plant is from the genus Lolium. In one embodiment, the plant is from the genus Festuca.

In one example, the plant from the genus Lolium is selected from the group consisting of the species: Lolium perenne; Lolium multiflorum; and Lolium x hybridum.

In one embodiment, the plant is a perennial, annual or hybrid fescue.

In one embodiment, the plant is from the genus Festuca.

In one embodiment, the plant from the genus Festuca is selected from the group consisting of the species: Festuca amethystina, Festuca arundinacea, Festuca cinerea, Festuca elegans, Festuca glauca, Festuca idahoensis, Festuca ovina, Festuca pallens, Festuca pratensis, Festuca rubra, Festuca rubra subsp. commutata, Festuca saximontana, and Festuca trachyphylla.

In various embodiments, the plant is of a species selected from the following: Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g., Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g., Brassica napus, Brassica rapa ssp. [canola, oilseed rape, turnip rape, kale]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g., Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g., Glycine max, Soja hispida or Soja max), Gossypium hirsutum, Flelianthus spp. (e.g., Flelianthus annuus), Hemerocaiiis fulva, Fiibiscus spp., Hordeum spp. (e.g., Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g., Lycopersicon esculentum, Lycopersicon lycopersicum, Lycopersicon pyriforme), Macrotyloma spp., Malus spp., Maipighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g., Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., SaNx sp., Sambucus spp., Secale cereale, Sesamum spp., Sinapis sp., Solanum spp. (e.g., Solanum tuberosum, Solan um betaceum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g., Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, and Ziziphus spp., among others.

In various embodiments, the grass is selected from the turf, forage or pasture grasses, such as the fescues (e.g., Festuca spp.,), the ryegrasses (e.g., Lolium spp.,), the bahiagrasses, the bentgrasses, the bermudagrasses, the bluegrasses, the buffalograsses, the centipedegrasses, St. Augustine grasses, and the zoysiagrasses.

In various embodiments, the grass is selected from the cereals or grain crops, such as but not limited to barley, maize (corn), millet, oats, rice, rye, sorghum, and wheat.

Further illustrative plants are monocotyledonous or dicotyledonous plants such as alfalfa, canola, cotton, flax, kapok, peanut, potato, soybean, sugarbeet, sugarcane, sunflower, tobacco, tomato, berry, fruit, legume, vegetable, for example, capsicum, a cucurbit such as cucumber, onion, ornamental plants, shrubs, cactuses, succulents, and trees.

In further illustrative embodiments, the plant may be any plant, including plants selected from the order Solanales, including plants from the following families: Convolvulaceae, Hydroleaceae, Montiniaceae, Solanaceae, and Sphenocleaceae, and plants from the order Asparagales, including plants from the following families: Amaryllidaceae, Asparagaceae, Asteliaceae, Blandfordiaceae, Boryaceae, Doryanthaceae, Hypoxidaceae, Iridaceae, Ixioliriaceae, Lanariaceae, Orchidaceae, Tecophilaeaceae, Xanthorrhoeaceae, and Xeronemataceae.

In another aspect the invention relates to a plant or part thereof comprising one or more epoxy-janthitrem compounds produced in accordance with the description herein, for example, one or more epoxy-janthitrem compounds produced by or using a host cell, expression construct, polynucleotide, or polypeptide as herein described.

In another aspect the invention relates to a plant or part thereof comprising one or more host cells, expression constructs, polynucleotides, or polypeptides as herein described.

In one embodiment the plant or part thereof is reproductively viable, for example, a seed, bulb or cutting or other plant part capable of propagation.

Accordingly, in various embodiments the invention relates to a combination of a plant or one or more plant cells, and one or more host cells, including for example a combination comprising a plant or one or more plant cells and one or more endophytic host cells such as one or more Epichloë cells, wherein the combination provides or comprises one or more epoxy-janthitrem compounds produced in accordance with the description herein, for example, one or more epoxy-janthitrem compounds produced by or using a host cell, expression construct, polynucleotide, or polypeptide as herein described.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7). These are only examples of what is specifically intended and all possible combinations of numerical values between the lowest value and the highest value enumerated are to be considered to be expressly stated in this application in a similar manner.

Those skilled in the art will appreciate the meaning of various terms of degree used herein. For example, as used herein in the context of referring to an amount (e.g., “about 9%”), the term “about” represents an amount close to and including the stated amount that still performs a desired function or achieves a desired result, e.g. “about 9%” can include 9% and amounts close to 9% that still perform a desired function or achieve a desired result. For example, the term “about” can refer to an amount that is within less than 10% of, within less than 5% of, within less than 1% of, within less than 0.1% of, or within less than 0.01% of the stated amount. It is also intended that where the term “about” is used, for example with reference to a figure, concentration, amount, integer or value, the exact figure, concentration, amount, integer or value is also specifically contemplated.

Other objects, aspects, features and advantages of the present invention will become apparent from the following description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

The invention is exemplified in the following non limiting embodiments and with reference to the accompanying figures.

### BRIEF DESCRIPTION OF THE FIGURES

FIG. 1 depicts the epoxy-janthitrem biosynthetic pathway as elucidated as described in Example 1 herein, with FIGS. 1A and 1B depicting in more detail different parts of the pathway along with the structural detail of the various compounds.

FIG. 2 depicts the derived amino acid sequence of the polypeptide encoded by the idtA gene from from Epichloë festucae var. lolii AR37 [SEQ ID NO: 3].

FIG. 3 depicts the derived amino acid sequence of the polypeptide encoded by the idtD gene from from Epichloë festucae var. lolii AR37 [SEQ ID NO: 6].

FIG. 4 depicts the derived amino acid sequence of the polypeptide encoded by the idtO gene from from Epichloë festucae var. lolii AR37 [SEQ ID NO: 9].

FIG. 5 depicts the derived amino acid sequence of the polypeptide encoded by the idtF gene from from Epichloë festucae var. lolii AR6 [SEQ ID NO: 17].

FIG. 6 depicts the derived amino acid sequence of the polypeptide encoded by the idtF gene from from Epichloë festucae var. lolii AR37 [SEQ ID NO: 19].

FIG. 7 depicts the derived amino acid sequence of the polypeptide encoded by the idtK gene from from Epichloë festucae var. lolii AR6 [SEQ ID NO: 21].

FIG. 8 depicts the derived amino acid sequence of the polypeptide encoded by the idtK gene from from Epichloë festucae var. lolii AR37 [SEQ ID NO: 23].

FIG. 9 depicts the amino acid sequence identity of polypeptides involved in the epoxy-janthitrem biosynthetic pathway from Epichloë festucae var. lolii AR1, Epichloë festucae var. lolii AR5, Epichloë festucae var. lolii AR6, Epichloë festucae var. lolii AR48, Epichloë festucae var. lolii AR3060, Epichloë festucae var. lolii E2368, Epichloë festucae var. lolii Fg1, and Epichloë festucae var. lolii F11, and Epichloë festucae var. lolii AR37, as described herein in Example 3.

FIG. 10 is a graph showing the results of a bioassay assessing the activity of purified epoxy-janthitrem I against Porina (Wiseana cervinata) larvae, as described herein in Example 4. The graph depicts weight change and square root transformed mean daily feeding scores for porina larvae fed diets containing five different concentrations of epoxyjanthitrem I over 7 days. Error bars are +SE.

FIG. 11 is a graph showing the results of a bioassay assessing the tremorgenicity of purified epoxy-janthitrem I and control compounds lolitrem B and paxilline in a mouse model, as described herein in Example 5. The graph depicts tremor score versus time for groups of mice (n=4) dosed intraperitoneally with paxilline (6 mg/kg), lolitrem B (2 mg/kg) or epoxyjanthitrem I (14 mg/kg). Tremor scores for the control group were zero at all time points. Error bars represent the standard error of the means.

FIG. 12 is a graph showing the results of a bioassay assessing the activity of purified epoxy-janthitrems against Porina (Wiseana copularis) larvae, as described herein in Example 6. The graph depicts average diet consumption per day (mg/larva) for porina larvae fed diets containing three different concentrations of epoxy-janthitrem I (EJ I), epoxy-janthitrem II (EJ II), epoxy-janthitrem III (EJ III), epoxy-janthitrem IV (EJ IV), epoxy-janthitriol (EJ Triol), and a combination of all epoxy-janthitrems at relative concentrations usually found in a plant (combo) over 21 days.

FIG. 13 is a graph showing the results of a bioassay assessing the tremorgenicity of purified epoxy-janthitrems in a mouse model, as described herein in Example 7. The graph depicts tremor score versus time for groups of mice (n=4) dosed intraperitoneally with epoxy-janthitrem I (EJ I, 13.5 mg/kg), epoxy-janthitrem II (EJ II, 14.2 mg/kg), epoxy-janthitrem III (EJ III, 17.2 mg/kg), epoxy-janthitrem IV (EJ IV, 16.5 mg/kg), epoxy-janthitriol (EJ triol, 16.2 mg/kg). Tremor scores for the control group were zero at all time points. Error bars represent the standard error of the means.

FIG. 14 is three sets of photos of PCR products verifying the presence of idtO (top panel), the idtD (middle panel), and the idtA (bottom panel) genes inserted into four Epichloë strains previously unable to produce epoxy-janithrem compounds, and the presence of endogenous idtO and idtD in positive control strain AR37 (left to right: AR584, AR3028, AR3056, AR1, and AR37), as described herein in Example 8.

FIG. 15 depicts the predicted truncated IdtD polypeptides resulting from the AR37 idtD g63 and g148 edits as described herein in Example 9. The sites of the g63 and g148 edits are shown with a solid arrow, and an outlined arrow, respectively, and substituted amino acids (compared to wild type) resulting from the edits are boxed.

FIG. 16 depicts the derived amino acid sequence of the polypeptide encoded by the edited idtD gene, AR37 idtD g63 edit [SEQ ID NO: 50], as describe in Example 9.

FIG. 17 depicts the derived amino acid sequence of the polypeptide encoded by the edited idtD gene, AR37 idtD g148 edit [SEQ ID NO: 51], as describe in Example 9.

FIG. 18 depicts the predicted truncated IdtA polypeptides resulting from the AR37 idtA g64 and g101 edits as described herein in Example 9. The sites of the g64 and g101 edits are shown with a solid arrow, and an outlined arrow, respectively, and substituted amino acids (compared to wild type) resulting from the edits are boxed.

FIG. 19 depicts the derived amino acid sequence of the polypeptide encoded by the edited idtA gene, AR37 idtD g64 edit [SEQ ID NO: 52], as describe in Example 9.

FIG. 20 depicts the derived amino acid sequence of the polypeptide encoded by the edited idtA gene, AR37 idtA g101 edit [SEQ ID NO: 53], as describe in Example 9.

FIG. 21 depicts the predicted truncated IdtO polypeptides resulting from the AR37 idtO g119 and g144 edits as described herein in Example 10, wherein: 1. AR37 IdtO (wt) polypeptide with the FAD binding domain indicated; 2. AR37 idtO g119 truncated polypeptide; 3. AR37 idtO g144 truncated polypeptide. The sites of the g119 and g144 edits are shown with a solid arrow, and an outlined arrow, respectively, and substituted amino acids (compared to wild type) resulting from the edits are boxed.

FIG. 22 depicts the derived amino acid sequence of the polypeptide encoded by the edited idtO gene, AR37 idtO g119 edit [SEQ ID NO: 70], as describe in Example 10.

FIG. 23 depicts the derived amino acid sequence of the polypeptide encoded by the edited idtO gene, AR37 idtO g144 edit [SEQ ID NO: 71], as describe in Example 10.

FIG. 24 depicts the predicted truncated IdtF polypeptides resulting from the AR37 idtF g119 single edit, two different idtA g64/idtF g119 double gene edits (strains AR37 idtA g64/idtF g119 #4 and AR37 idtA g64/idtF g119 #9), and an idtA g64/idtF g86 double gene edit (strain AR37 idtA g101/idtF g86 #8) as described herein in Example 10, wherein: 1. AR37 IdtF (wt) polypeptide with the Prenyl transferase domain indicated; 2. AR37 idtF g119 truncated polypeptide; 3. AR37 idtA g64/idtF g119 #4 truncated polypeptide; 4. AR37 idtA g64/idtF g119 #9 truncated polypeptide; 5. AR37 idtA g101/idtF g86 #8 truncated polypeptide. The sites of the g86 and g119 edits are shown with a solid arrow, and an outlined arrow, respectively, and substituted amino acids (compared to wild type) resulting from the edits are boxed.

FIG. 25 depicts the derived amino acid sequence of the polypeptide encoded by the edited idtF gene in the AR37 idtA g101/idtF g86 #8 double mutant [SEQ ID NO: 72], as describe in Example 10.

FIG. 26 depicts the derived amino acid sequence of the polypeptide encoded by the edited idtF gene in the AR37 idtF g119 #4 single mutant strain and in the AR37 idtA g64/idtF g119 #9 double mutant strain [SEQ ID NO: 73], as describe in Example 10.

FIG. 27 depicts the derived amino acid sequence of the polypeptide encoded by a second edited idtF gene in the AR37 idtA g64/idtF g119 #4 double mutant strain [SEQ ID NO: 74], as describe in Example 10.

### DETAILED DESCRIPTION

The present invention relates to the provision of certain useful epoxy-janthitrem compounds, such as to organisms or systems in which they have not previously been available, or in which they are present together with one or more less desirable activities. In particular, the present invention relates to methods of conferring on one or more host cells the ability to produce one or more epoxy-janthitrem compounds, and to methods of preparing and using such host cells, for example in the provision of a benefit to an organism, such as a plant. Polynucleotides encoding polypeptides involved in the biosynthesis of epoxy-janthitrem compounds, and such polypeptides, together with related entities, are also encompassed herein. The present invention provides for the first time the heterologous expression of genes in the epoxy-janthitrem gene cluster that encode enzymes catalysing the conversion and synthesis of epoxy-janthitrem precursors and compounds in the epoxy-janthitrem biosynthetic pathway, and thereby the production of one or more epoxy-janthitrem compounds in a heterologous host cell not previously able to produce such compounds.

It will be appreciated by those skilled in the art on reading this description that one or more beneficial activites can be conferred on a host cell or an organism that previously did not embody those benefits, but may have had one or more other desirable attributes. For example, in the context of endophytic fungi/plant symbionts, certain fungal strains may be particularly adept at colonising a plant, or of being competently transmitted to new generations of plant, for example via high transmission rates in seed, or of being environmentally stable, such as being cold- or heat-tolerant, or drought tolerant. Frequently, however, such strains will lack one or more other desirable attributes, such as the ability to confer a benefit on the plant, for example the ability to produce one or more epoxy-janthitrem compounds and the benefits attendant thereto. In certain aspects, the present invention relates to methods of addressing such deficiencies, for example to provide host cells, including those which have other favourable characteristics with one or more new characteristics, such as the ability to produce one or more epoxy-janthitrem compounds, preferably while retaining their pre-existing beneficial attributes.

Definitions

As used herein, the term “and/or” can mean “and” or “or”.

The term “agriculturally acceptable carrier” covers all liquid and solid carriers known in the art such as water and oils, as well as adjuvants, dispersants, binders, wettants, surfactants, humectants, protectants, UV protectants and/or stabilisers, tackifiers, and the like that are ordinarily known for use in the preparation of agricultural compositions, including insecticide compositions.

The terms “comprise”, “comprises”, and “comprising” as used in this specification and claims are not to be interpreted in an exclusive or exhaustive sense, and mean “consisting at least in part of”. When interpreting each statement in this specification that includes the term “comprise”, “comprises”, or “comprising”, features other than that or those prefaced by the term may also be present. Related terms such as “including”, “include” and “includes” are to be interpreted in the same manner.

The term “consisting essentially of” when used in this specification refers to the features stated and allows for the presence of other features that do not materially alter the basic characteristics of the features specified.

The term “consisting of” as used herein means the specified materials or steps of the claimed invention, excluding any element, step, or ingredient not specified in the claim.

The term “contacting” used herein in reference to a pest refers to the provision of a compound, composition, cell, or similar of the invention to a pest in a manner useful to effect pest control. Most commonly contacting will involve the pest feeding on material comprising a composition, cell, or compound of the invention but is not limited thereto. Accordingly, “contacting” includes feeding.

The term “control” or “controlling” as used herein in reference to a pest or pest population generally comprehends preventing an increase in, reducing, or eradicating a population or one or more members of a population, or preventing, reducing or eradicating infection or infestation by one or more pests or pathogens, such as infection by one or more phytopathogens or pests, or inhibiting the rate and extent of such infection, such as reducing a pest population at a locus, for example in or on a plant or its surround ings, wherein such prevention or reduction in the infection(s) or population(s) is statistically significant with respect to untreated infection(s) or population(s). Curative treatment is also contemplated. In certain particularly contemplated embodiments, such control is achieved by increased mortality amongst the pest or pathogen population.

It will be appreciated that control may be via antagonism, which may take a number of forms. In one form, the compounds, compositions, cells, or similar contemplated herein may simply act as a repellent. In another form, the compounds, compositions, cells, or similar contemplated herein may render the environment, such as a plant or its surroundings to which the compounds, compositions, cells, or similar contemplated herein are applied, unsuitable or unfavourable for the pest or pathogen. In one example, the compounds, compositions, cells, or similar contemplated herein deter feeding. In a further, preferred form, the compounds, compositions, cells, or similar contemplated herein may incapacitate, render infertile, impede the growth of, impede the spread or distribution of, and/or kill the pest or pathogen. Accordingly, the antagonistic mechanisms include but are not limited to antibiosis, immobilisation, infertility, and toxicity. Therefore, entities which act as antagonists of one or more pests, such that such entities are useful in the control of a pest, can be said to have pesticidal activity. For example, compounds, compositions, cells, or similar that act as antagonists of one or more insects can be said to have insecticidal efficacy. Furthermore, an agent or composition that is or comprises an antagonist of a pest can be said to be a pesticidal agent or a pesticidal composition, for example, an agent that is an antagonist of an insect can be said to be a pesticidal agent. Likewise, a composition that is or comprises an antagonist of an insect can be said to be an insecticidal composition.

Accordingly, as used herein a “pesticidal composition” is a composition which comprises or includes at least one agent that has pesticidal efficacy.

In various embodiments, said pesticidal efficacy is the ability to repel, incapacitate, render infertile, impede the growth of, or kill one or more pests, including one or more insects or nematodes, for example within 14 days of contact with the pest, such as within 7 days.

Accordingly, as used herein an “insecticidal composition” is a composition which comprises or includes at least one agent that has insecticidal efficacy.

The term “endogenous” as used herein with reference to a biological entity, such as a cell, tissue or organism, contemplates the characteristic of existing or occurring naturally in or with, or being produced naturally by or within, that entity. For example, endogenous expression refers to expression, for example of a gene or gene product, occurring naturally in a cell, tissue, or organism, while an endogenous polynucleotide refers to a polynucleotide naturally present, for example a polynucleotide that is naturally present in a cell, tissue or organism. It will be appreciated by those skilled in the art that “endogenous” may refer to any constituent of a biological entity, such as a biological system, a cell, a tissue or an organism, including but not limited to a polynucleotide, a polypeptide including a non-ribosomal polypeptide, a lipid, a fatty acid, a polyketide, a metabolite, and the like.

The term “exogenous” as used herein with reference to a biological entity, such as a cell, tissue or organism, contemplates the characteristic of not existing or occurring naturally in or with, or not being produced naturally by or within, that entity. For example, an exogenous polynucleotide refers to a polynucleotide that is not naturally present, for example a polynucleotide that is not naturally present in a biological entity, cell, tissue or organism, while an exogenous metabolite refers to a metabolite that is not naturally present, for example is not naturally produced by a cell, tissue or organism . It will be appreciated by those skilled in the art that “exogenous” may refer to any constituent of a biological entity, such as a biological system, a cell, a tissue or an organism, including but not limited to a polynucleotide, a polypeptide including a non-ribosomal polypeptide, a lipid, a fatty acid, a polyketide, a metabolite, and the like, that has been introduced into a biological entity, for example by expression of an exogenous polynucleotide or polypeptide within or by that entity.

The term “expression” as used herein refers to a process by which a gene produces a product, such as a biochemical, for example, an RNA or polypeptide. The process includes any manifestation of the functional presence of the gene within the cell including, without limitation, gene knockdown as well as both transient expression and stable expression. It includes, without limitation, transcription of the gene into messenger RNA (mRNA), transfer RNA (tRNA), small hairpin RNA (shRNA), small interfering RNA (siRNA), or any other RNA product, and the translation of such mRNA into polypeptide(s). If the final desired product is biochemical, expression includes the creation of that biochemical and any precursors.

The term “expression construct” refers to a genetic construct that includes elements that permit transcribing the polynucleotide molecule of interest, and, optionally, translating the transcript into a polypeptide. An expression construct typically comprises in a 5′ to 3′ direction:

(1) a promoter, functional in the host cell into which the construct will be introduced,

(2) the polynucleotide to be expressed, and

(3) a terminator functional in the host cell into which the construct will be introduced.

Expression constructs of the invention are inserted into a replicable vector for cloning or for expression, or are incorporated into the host genome.

The term “gene” as used herein refers to a basic unit of heredity, typically located at a definite position (locus) within the genome of an organism, and comprising a sequence of nucleotides the order of which determines the order of monomers in a polypeptide or polynucleotide gene product. The term “native gene” refers to a gene as found in nature with its own naturally-occurring regulatory sequences.

The term “gene cluster” as used herein refers to a group of genes located closely together on the same chromosome. Frequently, the genes in a gene cluster are co-regulated, and/or the gene products encoded by genes in a gene cluster participate in a particular cellular function, such as a particular aspect of cellular primary or secondary metabolism, cellular growth or regulation, apoptosis, or the like. In one representative example, a gene cluster comprises a group of genes the products of which each participate in a biochemical reaction that comprises part of a enzymatic pathway, for example, a pathway resulting in the biosynthesis of a primary metabolite or of a secondary metabolite, a pathway involving the catalysis of a particular metabolite, or the like.

The term “genetic construct” refers to a polynucleotide molecule, usually double-stranded DNA, which has been conjugated to another polynucleotide molecule. In one non-limiting example a genetic construct is made by inserting a first polynucleotide molecule into a second polynucleotide molecule, for example by restriction/ligation as known in the art. In some embodiments, a genetic construct comprises a single polynucleotide module, at least two polynucleotide modules, or a series of multiple polynucleotide modules assembled into a single contiguous polynucleotide molecule. Methods well known in the art, including for example PCR, are well adapted to the preparation of genetic constructs in accordance with the description provided herein.

The term “heterologous” as used herein with reference to a biological entity, such as a cell, tissue or organism, contemplates the characteristic of not existing or occurring naturally, or naturally in or with, or not being produced naturally by or within, that entity, in addition to the characteristic of existing or occurring naturally in or with, or being produced naturally by or within, that entity, but at a different locus, or under different regulation or control, or under different conditions, to that occurring naturally . For example, in one embodiment a heterologous polynucleotide is a polynucleotide that is not endogenous to the entity, for example, is not endogenous to the cell into which it has been introduced, but has been obtained from another entity or has been synthesised. In another embodiment, a heterologous polynucleotide is one which is endogenous, but is expressed from a different locus or altered in its expression, for example is under the control of a different promoter from that with which it is associated naturally. Generally, although not necessarily, heterologous polynucleotides and heterologous polypeptides are not normally produced by the cell or in the same way in the cell in which they are expressed. Thus, heterologous nucleic acid includes a nucleic acid molecule not present in the exact orientation or position as the counterpart nucleic acid molecule is found in the naturally occurring genome from which it is derived. It also can refer to a nucleic acid molecule from another organism or species (i.e., exogenous).

Similarly, when used with reference to polynucleotide regulatory elements, heterologous means a polynucleotide regulatory element that is not a native and naturally-occurring polynucleotide regulatory element, or a polynucleotide regulatory element that is not normally associated with the polynucleotide sequence with which it is operably linked. In certain examples, a heterologous regulatory element is operably linked to a polynucleotide of interest such that the polynucleotide of interest can be expressed when desired, for example from a vector, genetic construct, or within a host as contemplated herein. Such heterologous regulatory elements include promoters normally associated with other genes, ORFs or coding regions, and/or promoters isolated from any other bacterial, viral, eukaryotic, or mammalian cell.

Accordingly, the term “heterologous host” means an entity such as a host cell or organism that comprises one or more non-naturally-occurring characteristics. For example, a heterologous host cell will in certain embodiments comprise one or more heterologous nucleic acids and/or one or more heterologous polypeptides that are not normally associated with the host cell.

Heterologous expression refers to the expression in a host cell of a heterologous polypeptide, such as a polypeptide encoded by heterologous nucleic acid that has been introduced, such as by transformation, electroporation, transduction, modification, or any other means, into the host cell, or expression in a host cell of or from a heterologous polynucleotide.

The terms “heterologously expressing” and “heterologous expression” mean the expression of a heterologous gene product, of a heterologous polynucleotide, or of a heterologous polypeptide, in a host cell.

It will be appreciated that heterologous expression encompasses, but is not limited to exogenous expression, and will in certain embodiments comprise expression of an endogenous constituent in a non-naturrally occurring manner, such as from a non-naturally locus, or under different conditions, regulation, or controls than that occurring naturally.

The term “insecticide” as used herein refers to agents which act to kill or control the growth of insects, including insects at any developmental stage. The related term “insecticidal” will be understood accordingly.

The terms “modify”, “modified” and grammatical variants thereof as used herein in reference to the genetic material present in or the genome of a host cell or organism contemplates the use and/or result of any method of altering the naturally-occurring genetic material present in or the genome of said cell or organism. Methods to modify endogenous genetic material, such as genomic DNA, are well known in the art, and include targeted and non-targeted insertion, integration, and editing methods. For example, targeted genome editing methods using engineered nucleases (such as clustered, regularly interspaced, short palindromic repeat (CRISPR) technology), are amenable to use in the methods described here, for example for generating RNA-guided nucleases, such as Cas9, with targeted sequence specificities. Genome editing using such targeted techniques has been used to rapidly, easily and efficiently modify endogenous genes in a wide variety of cell types, and in organisms that have previously been intractable to genetic modification, or challenging to manipulate genetically.

The term “pest” as used herein refers to organisms that are of inconvenience to, or deleterious to, another organism, such as a plant or animal, whether directly or indirectly. In one embodiment the term refers to organisms that cause damage to animals, including humans, or plants. The damage may relate to plant or animal health, growth, yield, reproduction or viability, and may be cosmetic damage. In certain particularly contemplated embodiments, the damage is of commercial significance. As will be apparent from the context, the term “pest” as used herein will typically refer to one or more organisms that cause damage to plants, for example, cultivated plants, including horticulturally or agriculturally important plants.

The term “plant” as used herein encompasses not only whole plants, but extends to plant parts, cuttings as well as plant products including roots, shoots, leaves, bark, pods, flowers, seeds, stems, callus tissue, nuts and fruit, bulbs, tubers, corms, grains, cuttings, root stock, or scions, and includes any plant material whether pre-planting, during growth, and at or post harvest. Plants that may benefit from the application of the present invention cover a broad range of agricultural and horticultural crops. Particularly contemplated plants that may benefit from the application of certain aspects of compounds, compositions, cells, polynucleotides and polypeptides described herein are grasses.

The term “plant derived materials” refers to products that may be produced from a plant or part thereof. It will be appreciated that a person skilled in the art will know of various examples of plant derived products, such as hay, silage or other types of feed or products.

The term “polynucleotide(s),” as used herein, means a single or double-stranded deoxyribonucleotide or ribonucleotide polymer of any length, and include as non-limiting examples, coding and non-coding sequences of a gene, sense and antisense sequences, exons, introns, genomic DNA, cDNA, pre-mRNA, mRNA, rRNA, siRNA, miRNA, tRNA, ribozymes, recombinant polynucleotides, isolated and purified naturally occurring DNA or RNA sequences, synthetic RNA and DNA sequences, nucleic acid probes, primers, fragments, genetic constructs, vectors and modified polynucleotides. Reference to nucleic acids, nucleic acid molecules, nucleotide sequences and polynucleotide sequences is to be similarly understood. It will be appreciated that a wide variety of synthetic and/or non-naturally occurring nucleotide analogues are available, such that polynucleotides comprising one or more of said synthetic or non-naturally occurring nucleotide analogues can be prepared. The use of such polynucleotides in the methods and compositions described herein is likewise contemplated.

The term “surroundings” when used in reference to a plant subject to the methods and compositions of the present invention includes water, leaf litter, and/or growth med is adjacent to or a round the plant or the roots, tubers or the like thereof, adjacent plants, cuttings of said plant, supports, water to be administered to the plant, and coatings includ ing seed coatings. It further includes storage, packaging or processing materials such as protective coatings, boxes and wrappers, and planting, maintenance or harvesting equipment.

The term “vector” as used herein refers to a polynucleotide molecule, usually but not limited to a double stranded DNA, which is amenable to use in molecular biological techniques, for example to modify, manipulate, replicate, amplify, or transport a polynucleotide molecule. In certain embodiments, a vector is used to transport a polynucleotide molecule, such as but not limited to a genetic construct, for example an expression construct, into a host cell or organism. In certain examples the vector is capable of replication and/or maintenance in more than one host system.

Various aspects of the invention are described in further detail in the following subsections. Unless otherwise defined, all technical and scientific terms used herein have the same meaning as is commonly understood by one of ordinary skill in the art to which this invention belongs. In case of conflict, the present specification, including definitions, will prevail. Methods and materials similar or equivalent to those described herein can be used in the practice of the invention, and examples of suitable methods and materials are described below. The materials, methods, and examples described herein are illustrative only and are not intended to be limiting.

Epoxy-Janthitrem Synthesis

Certain Epichloë are capable of synthesising a class of indole diterpene compounds referred to herein as epoxy-janthitrem compounds or epoxy-janthitrems.

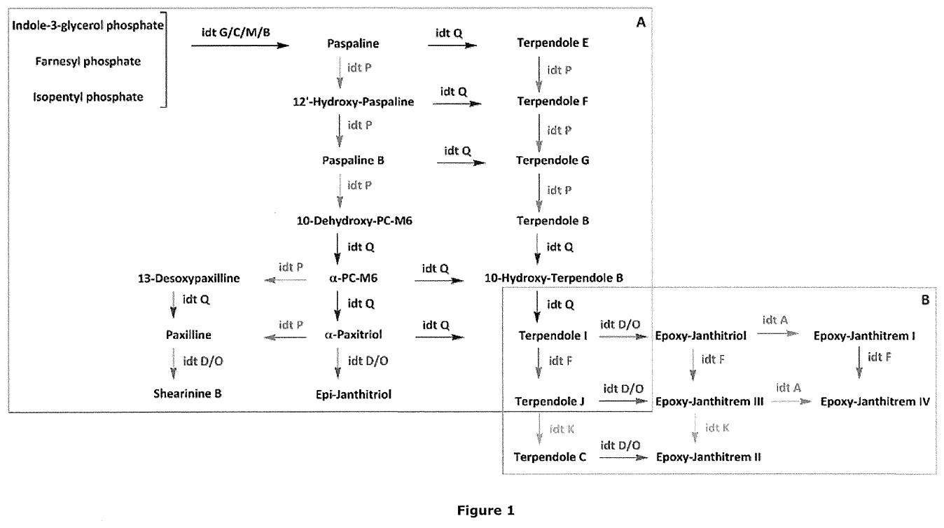

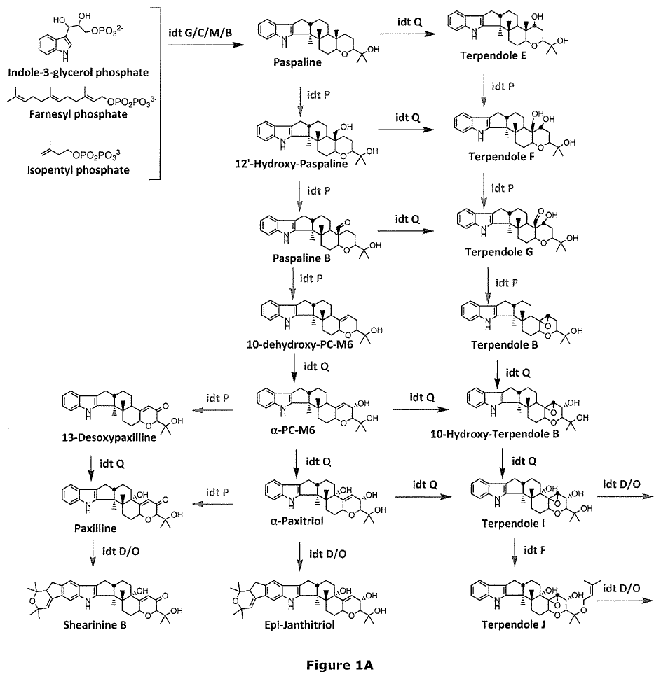

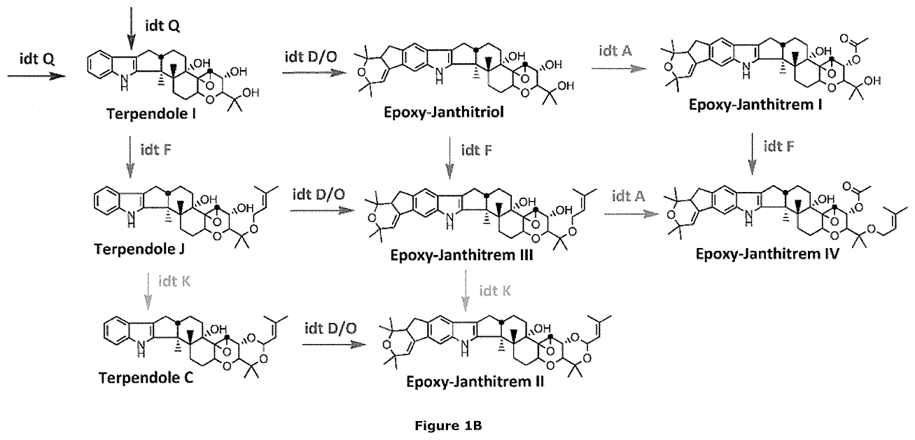

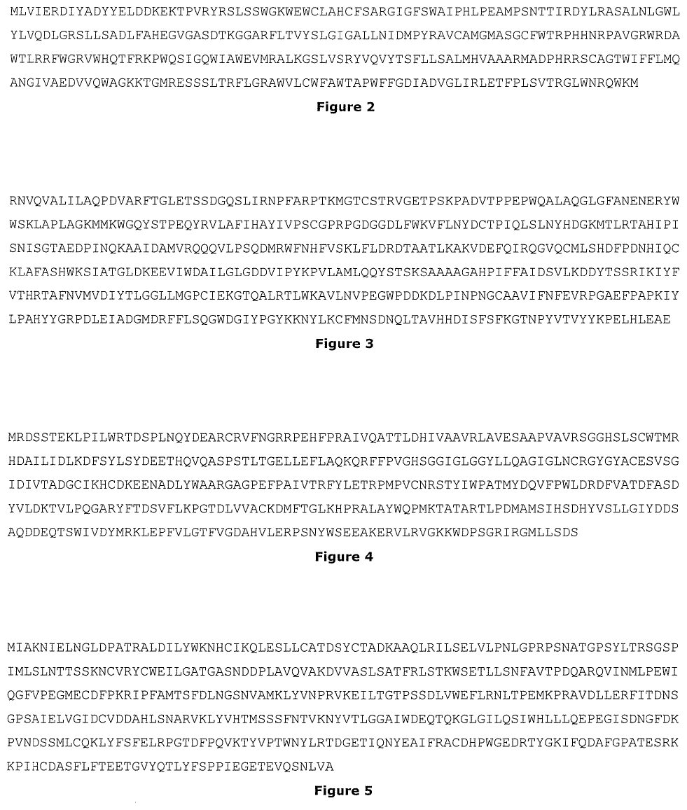

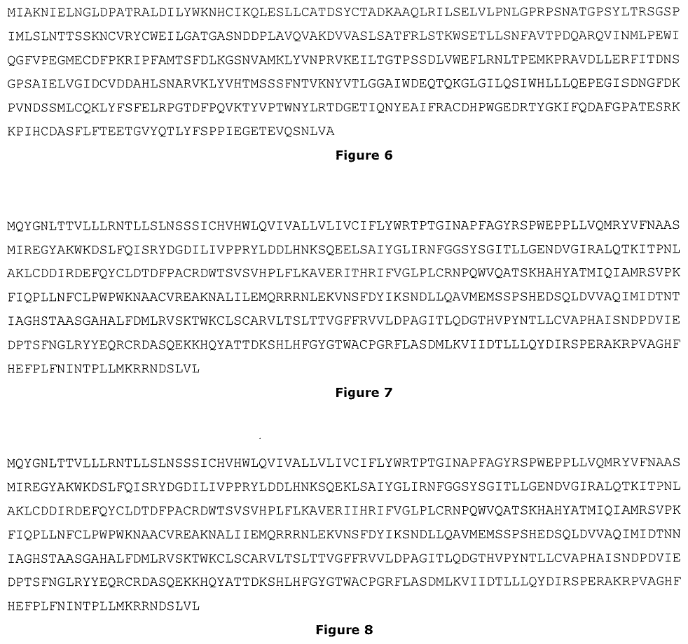

As discussed herein in the examples and as shown in FIG. 1, the biosynthetic pathway involved in the production of epoxy-janthitrem compounds begins with the indole diterpene precursors farnesyl pyrophosphate, isopentyl pyrophosphate, and indole-3-glycerol phosphate. Through the activity of enzymes involved in the indole diterpene pathway and encoded by multiple genes in the indole diterpene gene cluster, the compound terpendole I is produced.

The genes idtD and idtO encode a prenyl transferase homolog, and a FAD dependent oxygenase homolog, respectively, which together catalyse the first step in the epoxy-janthitrem branch of the IDT pathway. Accordingly, while it will be recognised that each of the compounds preceding terpendole I in the IDT pathway, for example each of terpendole C, terpendole J, terpendole B, terpendole G, terpendole F, terpendole E, α-paxitriol, α-PC-M6, paspaline B, 12′-hydroxy-paspaline, paspaline, and emindole SB (see FIG. 1) are considered precursors to an epoxy-janthitrem compound, terpendole I can be considered the immediate precursor to the epoxy-janthitrem biosynthetic pathway. Accordingly, in certain embodiments, the genes of particular interest herein, for example including, but not limited to idtA, idtD, and idtO, are those encoding a polypeptide that catalyses the conversion of a substrate in the epoxy-janthitrem biosynthetic pathway from terpendole I onwards. It will be understood from the elaboration of the epoxy-janthitrem biosynthetic pathway provided herein that further genes of particular interest that encode a polypeptide that catalyses the conversion of a substrate in the epoxy-janthitrem biosynthetic pathway include idtF and idtK.

IdtD is predicted to add two prenyl groups to C21 and C22 of the terpendole precursor, while IdtO is predicted to circularise these two prenyl groups, together producing epoxy-janthitriol.

The gene idtA encodes an acyltransferase homolog, which is predicted to convert epoxy-janthitriol to epoxy-janthitrem I and epoxy-janthitrem III to epoxy-janthitrem IV by the addition of an acyl group.

In certain embodiments, the epoxy-janthitrem compound is a compound of formula I

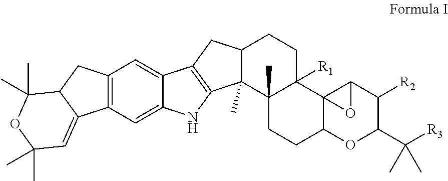

wherein R₁, R₂, and R₃ are each independently absent or selected from H, CH₂, CH₃, OH, O, COOH, OCOCH₃, C₁-C₆ straight chain or branched alkyl, OCH₂CHC(CH₃)₂, (O)₂CHCHC(CH₃)₂.

In various embodiments, R₂ is is (O)₂CHCHC(CH₃)₂, and/or R₃ is (O)₂CHCHC(CH₃)₂. For example, both R₂ and R₃ are (O)₂CHCHC(CH₃)₂.

In various embodiments, R₁, R₂, and R₃ are each independently selected from H, CH₂, CH₃, OH, and O.

In one embodiment, R₁, R₂, and R₃ are OH (Compound IA).

In one embodiment, R₁ and R₃ are OH, and R₂ is OCOCH₃ (Compound IB).

In one embodiment, R₁ is OH, R₂ and R₃ are (O)₂CHCHCH(CH₃)₂ (Compound IC).

In one embodiment, R₁ and R₂ are OH, and R₃ is OCH₂CHC(CH₃)₂ (Compound ID).

In one embodiment, R₁ is OH, R₂ is OCOCH₃, and R₃ is OCH₂CHC(CH₃)₂ (Compound IE).

Representative epoxy-janthitrem compounds include the following:

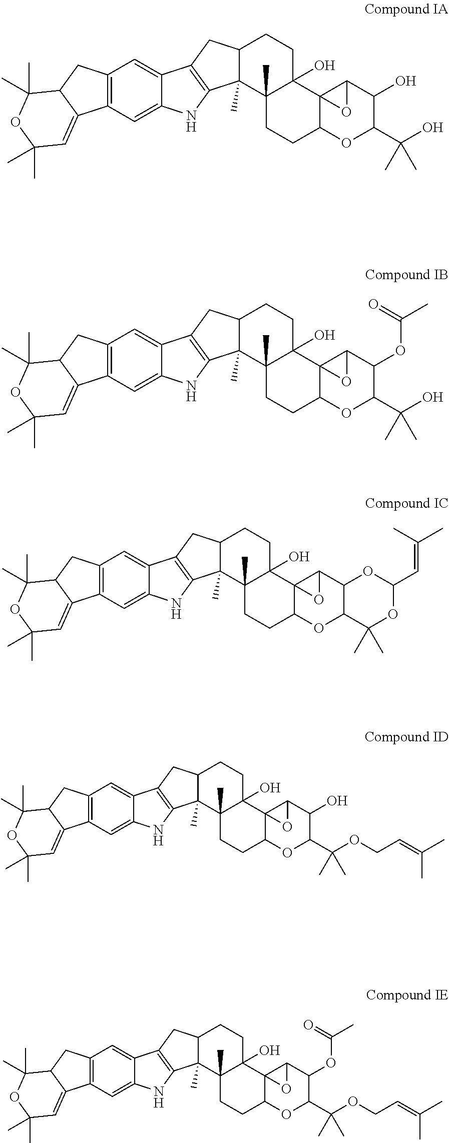

In certain embodiments, the epoxy-janthitrem compound is a compound of formula II

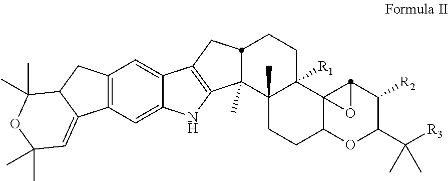

wherein R₁, R₂, and R₃ are each independently absent or selected from H, CH₂, CH₃, OH, O, COOH, OCOCH₃, C₁-C₆ straight chain or branched alkyl, OCH₂CHC(CH₃)₂, (O)₂CHCHC(CH₃)₂.

In various embodiments, R₂ is is (O)₂CHCHC(CH₃)₂, and/or R₃ is (O)₂CHCHC(CH₃)₂. For example, both R₂ and R₃ are (O)₂CHCHC(CH₃)₂.

In various embodiments, R₁, R₂, and R₃ are each independently selected from H, CH₂, CH₃, OH, and O.

In one embodiment, R₁, R₂, and R₃ are OH (Compound IIA, epoxy-janthitriol).

In one embodiment, R₁ and R₃ are OH, and R₂ is OCOCH₃ (Compound IIB, epoxy-janthitrem I). In one embodiment, R₁ is OH, R₂ and R₃ are (O)₂CHCHCH(CH₃)₂ (Compound IIC, epoxy-janthitrem II).

In one embodiment, R₁ and R₂ are OH, and R₃ is OCH₂CHC(CH₃)₂ (Compound IID, epoxy-janthitrem III).

In one embodiment, R₁ is OH, R₂ is OCOCH₃, and R₃ is OCH₂CHC(CH₃)₂ (Compound IIE, epoxy-janthitrem IV).

Specifically contemplated epoxy-janthitrem compounds include the following:

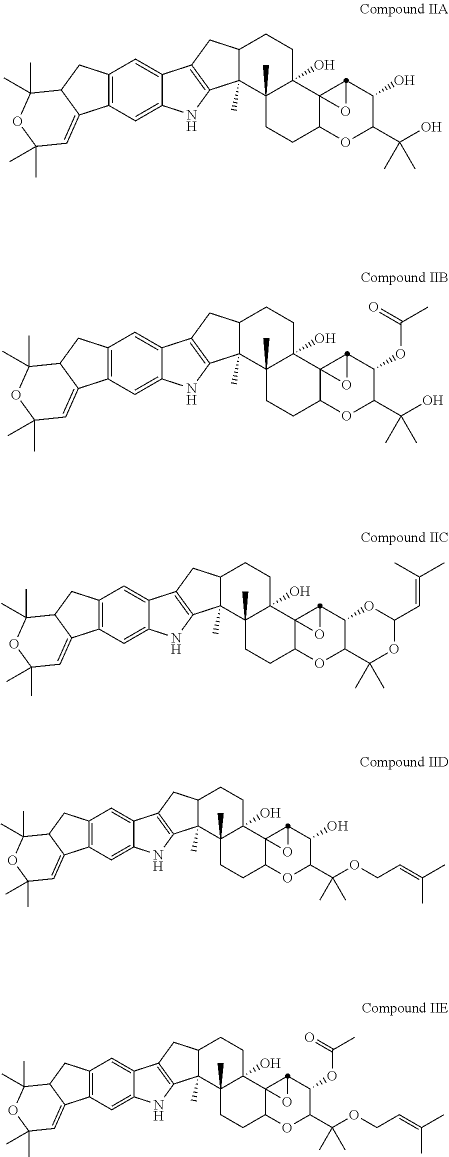

In certain embodiments, the epoxy-janthitrem compound is a compound of formula III

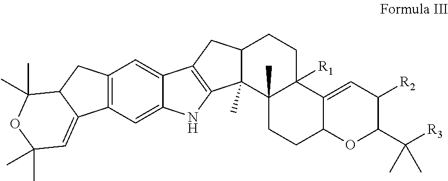

wherein R₁, R₂, and R₃ are each independently absent or selected from H, CH₂, CH₃, OH, O, COOH, OCOCH₃, C₁-C₆ straight chain or branched alkyl, OCH₂CHC(CH₃)₂, (O)₂CHCHC(CH₃)₂.

In various embodiments, R₂ is is (O)₂CHCHC(CH₃)₂, and/or R₃ is (O)₂CHCHC(CH₃)₂. For example, both R₂ and R₃ are (O)₂CHCHC(CH₃)₂.

In various embodiments, R₁, R₂, and R₃ are each independently selected from H, CH2, CH₃, OH, and O.

In one embodiment, R₁, R₂, and R₃ are OH (Compound IIIA).

In one embodiment, R₁ and R₃ are OH, and R₂ is 0 (Compound IIIB).

Representative epoxy-janthitrem compounds include the following:

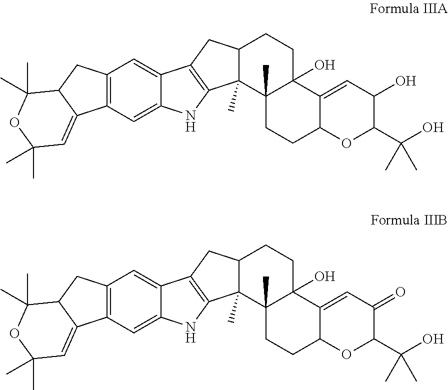

In certain embodiments, the janthitrem or epoxy-janthitrem compound is a compound of formula IV

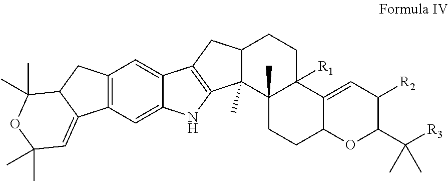

wherein R₁, R₂, and R₃ are each independently absent or selected from H, CH2, CH₃, OH, O, COOH, OCOCH₃, C₁-C₆ straight chain or branched alkyl, OCH₂CHC(CH₃)₂, (O)₂CHCHC(CH₃)₂.

In various embodiments, R₂ is is (O)₂CHCHC(CH₃)₂, and/or R₃ is (O)₂CHCHC(CH₃)₂. For example, both R₂ and R₃ are (O)₂CHCHC(CH₃)₂.

In various embodiments, R₁, R₂, and R₃ are each independently selected from H, CH2, CH₃, OH, and O.

In one embodiment, R₁, R₂, and R₃ are OH (Compound IVA, Epi-Janthitriol).

In one embodiment, R₁ and R₃ are OH, and R₂ is O (Compound IVB, Shearinine B).

Specifically contemplated epoxy-janthitrem compounds include the following:

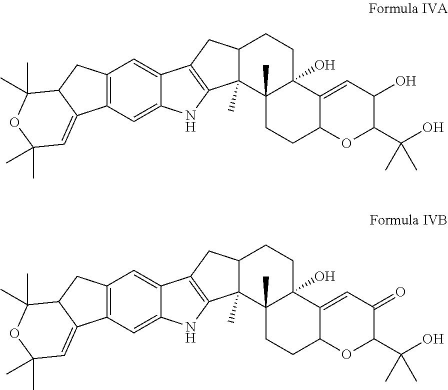

In certain embodiments, the epoxy-janthitrem precursor compound is a compound of formula V

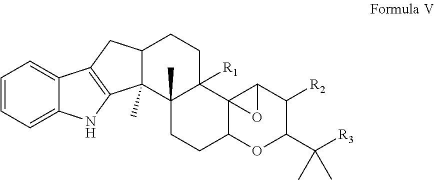

wherein R₁, R₂, and R₃ are each independently selected from H, CH₃, OH, O, COOH, C₁-C₆ straight chain or branched alkyl, OCH₂CHC(CH₃)₂, (O)₂CHCHC(CH₃)₂.

In various embodiments of the compound of formula V, R₁, R₂, and R₃ are each independently selected from H, OH, OCH₂CHC(CH₃)₂, and O(O)CHCHC(CH₃)₂.

In various embodiments of the compound of formula V, R₂ is is (O)₂CHCHC(CH₃)₂, and/or R₃ is (O)₂CHCHC(CH₃)₂. For example, both R₂ and R₃ are (O)₂CHCHC(CH₃)₂.

In one embodiment, R₁, R₂, and R₃ are each OH (Compound VA).

In one embodiment, R₁, and R₂ are each OH, and R₃ is OCH₂CHC(CH₃)₂ (Compound VB).

In one embodiment, R₁ is OH, and R₂ and R₃ are (O)₂CH₂CHC(CH₃)₂ (Compound VC).

Representative epoxy-janthitrem precursor compounds include the following:

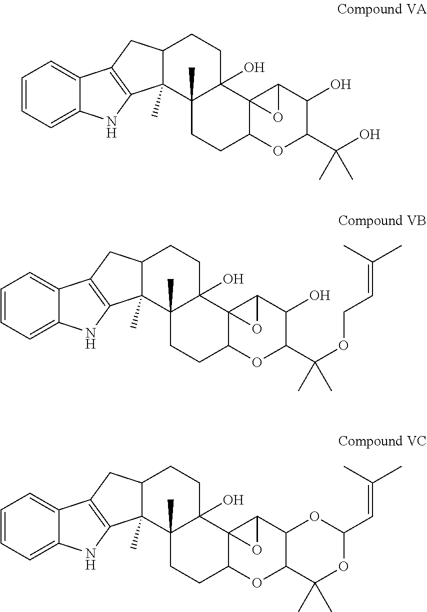

In certain embodiments, the epoxy-janthitrem precursor compound is a compound of formula VI

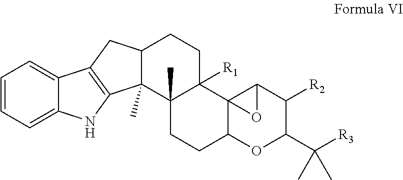

wherein R₁, R₂, and R₃ are each independently selected from H, CH₃, OH, O, COOH, C₁-C₆ straight chain or branched alkyl, OCH₂CHC(CH₃)₂, (O)₂CHCHC(CH₃)₂.

In various embodiments of the compound of formula VI, R₁, R₂, and R₃ are each independently selected from H, OH, OCH₂CHC(CH₃)₂, and O(O)CHCHC(CH₃)₂.

In various embodiments of the compound of formula VI, R₂ is is (O)₂CHCHC(CH₃)₂, and/or R₃ is (O)₂CHCHC(CH₃)₂. For example, both R₂ and R₃ are (O)₂CHCHC(CH₃)₂.

In one embodiment, R₁, R₂, and R₃ are each OH (Compound VIA, terpendole I).

In one embodiment, R₁, and R₂ are each OH, and R₃ is OCH₂CHC(CH₃)₂ (Compound VIB, terpendole J).

In one embodiment, R₁ is OH, and R₂ and R₃ are (O)₂CH₂CHC(CH₃)₂ (Compound VIC, terpendole C).

Specifically contemplated epoxy-janthitrem precursor compounds include the following:

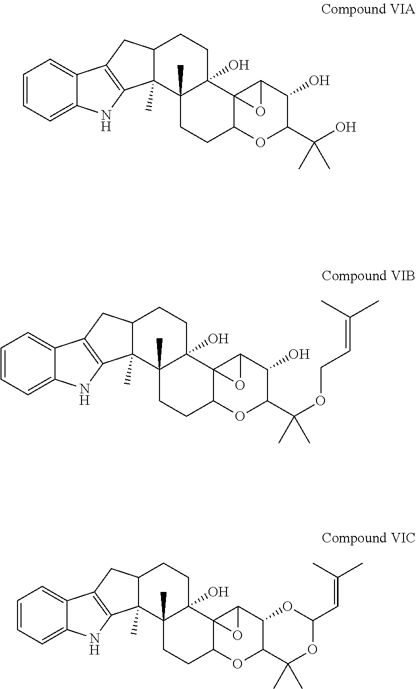

In certain embodiments, the epoxy-janthitrem precursor compound is a compound of formula VII

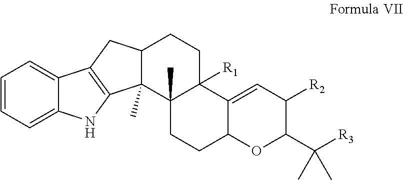

wherein R₁, R₂, and R₃ are each independently absent or selected from H, CH₃, OH, O, COOH.

In various embodiments of the compound of formula VII, R₁, R₂, and R₃ are each independently selected from H, OH, O.

In one embodiment, R₁ is H, and R₂, and R₃ are each OH (Compound VIIA).

In one embodiment, R₁, R₂, and R₃ are each OH (Compound VIIB).

In one embodiment, R₁ is H, and R₂ is O.

In one embodiment, R₁ is H, and R₂ is O, and R₃ is OH (Compound VIIC, 13-Desoxypaxilline).

In one embodiment, R₁, and R₃ are each OH, and R₂ is O (Compound VIID). Representative epoxy-janthitrem precursor compounds include the following:

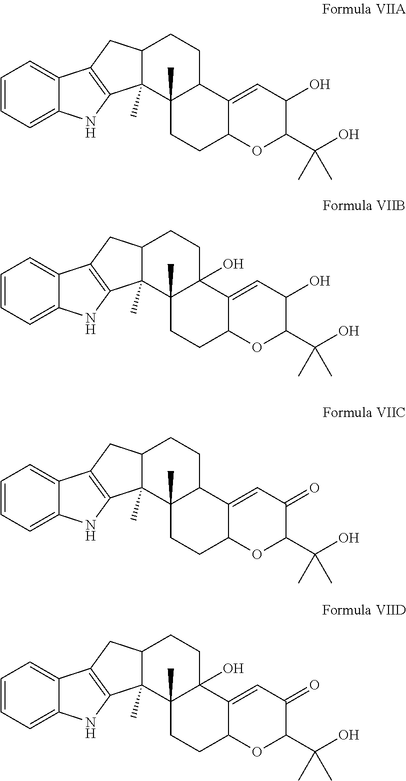

In certain embodiments, the epoxy-janthitrem precursor compound is a compound of formula VIII

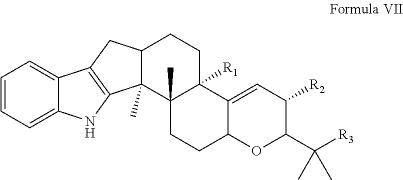

wherein R₁, R₂, and R₃ are each independently absent or selected from H, CH₃, OH, O, COOH.

In various embodiments of the compound of formula VIII, R₁, R₂, and R₃ are each independently selected from H, OH, O.

In one embodiment, R₁ is H, and R₂, and R₃ are each OH (Compound VIIIA, a-PC-M6).

In one embodiment, R₁, R₂, and R₃ are each OH (Compound VIIIB, α-Paxitriol).

In one embodiment, R₁ is H, and R₂ is O.

In one embodiment, R₁, and R₃ are each OH, and R₂ is O (Compound VIIIC, Paxilline). Specifically contemplated epoxy-janthitrem precursor compounds include the following:

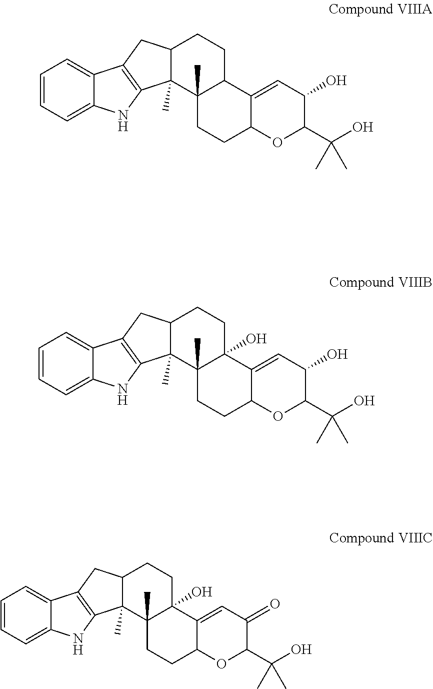

As described herein in the examples, the capability to produce one or more epoxy-janthitrem compounds including those described above has for the first time been conferred on a host cell not previously able to produce epoxy-janthitrem compounds. Accordingly, the provision to a heterologous host of one or more bioactivities associated with these compounds has been achieved. This capacity generally involves the expression in the host cell of one or more polypeptides involved in epoxy-janthitrem biosynthesis.

Polypeptides

As will be appreciated from this disclosure, polypeptides useful in the biosynthesis of one or more epoxy-janthitrems are provided herein. These include full length polypeptides, such as the polypeptides comprising the amino acid sequences depicted in Sequence ID NO: 3, 6, 9, 17, 19, 21, and 23, and functional domains present in those polypeptides, such as those identified herein and in the accompanying sequence identity listing, and variants of such polypeptides and functional domains.

In one embodiment, the polypeptide comprising the amino acid sequences depicted in SEQ ID NO: 3 or a functional variant or functional domain thereof has acyltransferase activity.

In one embodiment, the polypeptide comprising the amino acid sequences depicted in SEQ ID NO: 6 or a functional variant or functional domain thereof has prenyl transferase activity.

In one embodiment, the polypeptide comprising the amino acid sequences depicted in SEQ ID NO: 9 or a functional variant or functional domain thereof has oxygenase activity, for example FAD-dependent oxygenase activity.

In one embodiment, the polypeptide comprising the amino acid sequences depicted in SEQ ID NO: 17 or 19 or a functional variant or functional domain thereof has prenyl transferase activity.

In one embodiment, the polypeptide comprising the amino acid sequences depicted in SEQ ID NO: 21 or 23 or a functional variant or functional domain thereof has oxygenase activity, for example cytochrome P450 oxygenase activity.

In various embodiments, a polypeptide as contemplated herein catalyzes a biochemical reaction in the epoxy-janthitrem biosynthetic pathway leading from terpendole I onwards, for example, from terpendole Ito any one or more of epoxy-janthitrem I, epoxy-janthitrem II, epoxy-janthitrem III, epoxy-janthitrem IV, or epoxy-janthitriol. In various examples, the polypeptide is an acyltransferase, a prenyl transferase, or an oxygenase such as a FAD-dependent oxygenase or a cytochrome P450 oxygenase. In one example, the acyltransferase is IdtA, such as a polypeptide comprising the amino acid sequence presented in SEQ ID NO: 3, or is encoded by the idtA gene, such as that presented in SEQ ID NO:1 or SEQ ID NO: 2. In one example, the prenyl transferase is IdtD, such as a polypeptide comprising the amino acid sequence presented in SEQ ID NO: 6, or is encoded by the idtD gene, such as that presented in SEQ ID NO: 4 or SEQ ID NO: 5. In one example, the prenyl transferase is IdtF, such as a polypeptide comprising the amino acid sequence presented in SEQ ID NO: 17 or 19, or is encoded by the idtF gene, such as that presented in SEQ ID NO: 16 or 18. In one example, the FAD-dependent oxygenase is IdtO, such as a polypeptide comprising the amino acid sequence presented in SEQ ID NO: 9, or is encoded by the idtO gene, such as that presented in SEQ ID NO: 7 or SEQ ID NO: 8. In one example, the cytochrome P450 oxygenase is IdtK, such as a polypeptide comprising the amino acid sequence presented in SEQ ID NO: 21 or SEQ ID NO: 23, or is encoded by the idtK gene, such as that presented in SEQ ID NO: 20 or SEQ ID NO: 22.

In various embodiments, one or more of the polypeptides described above comprises a fusion polypeptide. For example, a fusion polypeptide as contemplated herein will in certain embodiments comprise one or more functional domains derived from, comprising or consisting of one of the sequences presented herein, such as an acyltransferase domain such as that presented in SEQ ID NO: 3, fused to another amino acid sequence to provide a fusion polypeptide.

Those skilled in the art will recognise, on reading this description, that these proteins can be considered representative examples of the polypeptides involved in the biosynthesis of one or more epoxy-janthitrem compounds suitable for use as contemplated herein. As such, various uses of and for these polypeptides, particularly in pest control methods comprising the provision of epoxy-janthitrem production to one or more host cells or organisms, are provided.

Proteins suitable for use herein include naturally-occurring proteins and peptides, and derivatives thereof including proteins and peptides having one or more amino acid variations from a naturally-occurring protein or peptide.

The term “amino acid” refers to natural amino acids, non-natural amino acids, and amino acid analogues. Unless otherwise indicated, the term “amino acid” includes both D and L stereoisomers if the respective structure allows such stereoisomeric forms.

Natural amino acids include alanine (Ala or A), arginine (Arg or R), asparagine (Asn or N), aspartic acid (Asp or D), cysteine (Cys or C), glutamine (Gin or Q), glutamic acid (Glu or E), glycine (Gly or G), histidine (His or H), isoleucine (He or I), leucine (Leu or L), Lysine (Lys or K), methionine (Met or M), phenylalanine (Phe or F), proline (Pro or P), serine (Ser or S), threonine (Thr or T), tryptophan (Tip or W), tyrosine (Tyr or Y) and valine (Val or V).

Non-natural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6- aminocaproic acid, 2-aminoheptanoic acid, 2-aminoisobutyric acid, 3-aminoisbutyric acid, 2- aminopimelic acid, tertiary-butylglycine (“tBuG”), 2,4-diaminoisobutyric acid, desmosine, 2,2′-diaminopimelic acid, 2,3-diaminopropionic acid, N-ethyl glycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), Nalkylglycine (“NAG”) including N-methylglycine, N- methylisoleucine, N-alkylpentylglycine (“NAPG”) including N-methylpentylglycine. N- methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analogue” refers to a natural or non-natural amino acid where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analogue of aspartic acid; N-ethylglycine is an amino acid analogue of glycine; or alanine carboxamide is an amino acid analogue of alanine. Other amino acid analogues include methionine sulfoxide, methionine sulfone, S-(carboxymethyl)-cysteine, S-(carboxymethyl) cysteine sulfoxide and S-(carboxymethyl)-cysteine sulfone.

A “fragment” of a polypeptide is a subsequence of the polypeptide, typically one that performs a function that is required for activity, such as enzymatic or binding activity, and/or provides a three-dimensional structure of the polypeptide or a part thereof, such as an epitope. It will be appreciated that a fragment of a polypeptide may possess or elicit a different function or functions from that possessed or exhibited by the full-length polypeptide from which it is derived.

As used herein, the term “peptide” refers a short polymer of amino acids linked together by peptide bonds. While it will be recognised that the names associated with various classes of amino acid polymers (e.g., peptides, proteins, polypeptides, etc.) are somewhat arbitrary, peptides are generally of about 50 amino acids or less in length. A peptide can comprise natural amino acids, non-natural amino acids, amino acid analogues, and/or modified amino acids. A peptide can be a subsequence of naturally occurring protein or a non-natural, including a synthetic, sequence.

As used herein, the term “synthetic peptide” encompasses a peptide having a distinct amino acid sequence from those found in natural peptides and/or proteins. A “synthetic peptide,” as used herein, can be produced or synthesized by any suitable method (e.g., recombinant expression, chemical synthesis, enzymatic synthesis, etc.), and can include any chemical modification to a parent peptide, and may include, but is not limited to such methods as truncations, deletions, cyclization or non-peptidic synthetic or semi-synthetic derivatives that retain the same biological function(s) as the starting peptide. Methods of protein synthesis, such as solid-state synthesis, are well known in the art.

The terms “peptide mimetic” or “peptidomimetic” refer to a peptide-like molecule that emulates a sequence derived from a protein or peptide. A peptide mimetic or peptidomimetic can contain amino acids and/or non-amino acid components. Examples of peptidomimetics include chemically modified peptides, peptoids (side groups are appended to the nitrogen atom of the peptide backbone, rather than to the α-carbons), β-peptides (amino group bonded to the β carbon rather than the α-carbon), etc. Chemical modification includes one or more modifications at amino acid side groups, α-carbon atoms, terminal amine group, or terminal carboxy group. A chemical modification can be adding chemical moieties, creating new bonds, or removing chemical moieties. Modifications at amino acid side groups include, without limitation, acylation of lysine ε-amino groups, N-alkylation of arginine, histidine, or lysine, alkylation of glutamic or aspartic carboxylic acid groups, lactam formation via cyclization of lysine ε-amino groups with glutamic or aspartic acid side group carboxyl groups, hydrocarbon “stapling” (e.g., to stabilize alpha-helix conformations), and deamidation of glutamine or asparagine. Modifications of the terminal amine group include, without limitation, the desamino, N-lower alkyl, N-di-lower alkyl, constrained alkyls (e.g. branched, cyclic, fused, adamantyl) and N-acyl modifications. Modifications of the terminal carboxy group include, without limitation, the amide, lower alkyl amide, constrained alkyls (e.g. branched, cyclic, fused, adamantyl) alkyl, dialkyl amide, and lower alkyl ester modifications. Lower alkyl is C1-C4 alkyl. Furthermore, one or more side groups, or terminal groups, can be protected by protective groups known to the ordinarily skilled peptide chemist. The a-carbon of an amino acid can be mono- or dimethylated.

It will be appreciated that any one of the proteins or peptides described herein in certain embodiments comprises one or more non-naturally occurring amino acids, one or more amino acid analogues, or is or comprises a synthetic peptide or polypeptide or a peptide mimetic. Similarly, it will be appreciated that any one of the proteins or peptides described herein will in certain embodiments be the starting point for one or more modifications, synthetic methods, or protein engineering methods to develop a peptide analogue having a desired biological activity—for example, a qualitatively similar bioactivity as the parent protein or peptide, but an effect of a quantitatively different magnitude, or indeed a different bioactivity from that elicited by the parent protein or peptide.

The term “fusion polypeptide”, as used herein, refers to a polypeptide comprising two or more amino acid sequences, for example two or more polypeptide domains, fused through respective amino and carboxyl residues by a peptide linkage to form a single continuous polypeptide. It should be understood that the two or more amino acid sequences can either be directly fused or indirectly fused through their respective amino and carboxyl terminii through a linker or spacer or an additional polypeptide.

The term “polypeptide”, as used herein, encompasses amino acid chains of any length but typically at least 10 amino acids, including full-length proteins, in which amino acid residues are linked by covalent peptide bonds. Polypeptides described herein are in certain embodiments purified natural products, or in other embodiments are produced partially or wholly using recombinant or synthetic techniques. The term may refer to a polypeptide, an aggregate of a polypeptide such as a dimer or other multimer, a fusion polypeptide, a polypeptide variant, or derivative thereof.

It will be understood that, for the particular polypeptides and proteins contemplated herein, natural variations can exist between individual strains or species. These variations may be demonstrated by (an) amino acid difference(s) in the overall sequence or by deletions, substitutions, insertions, inversions or additions of (an) amino acid(s) in said sequence. Amino acid substitutions which do not essentially alter biological and/or immunological activities, are well known. Amino acid replacements between related amino acids or replacements which have occurred frequently in evolution are, inter alia, Ser/Ala, Ser/Gly, Asp/Gly, Asp/Asn, Ile/Val. Other amino add substitutions include Asp/Glu, Thr/Ser, Ala/Gly, Ala/Thr, Ser/Asn, Ala/Val, Thr/Phe, Ala/Pro, Lys/Arg, Leu/Ile, Leu/Val and Ala/Glu. Based on this information, methods for rapid and sensitive protein comparison and determining the functional similarity between homologous proteins were developed. Such amino acid substitutions of the exemplary embodiments described herein, as well as variations having deletions and/or insertions are within the scope of the invention as long as the resulting proteins retain useful biological activity, for example, immunological reactivity. A protein comprising one or more variations in the amino acid sequence relative to that of a certain protein described herein, but that still provide a protein having useful biological activity compared to that of the protein specifically identified herein, are considered functional equivalents. For example, those variations in the amino acid sequence of a certain protein described herein that still provide a protein capable of reacting with an antibody specific to a protein specifically identified herein are considered as immunological functional equivalents of the proteins identified herein, and as such do not essentially influence the immunogenicity of the protein.

When a protein is used for example for agricultural, diagnostic or therapeutic purposes, for example for mediating a biological effect, for example one or more of the biological functions associated with the native protein in vivo, while it can be expedient to do so it is not necessary to use the whole protein. It is also possible to use a polypeptide fragment of that protein (as such or coupled to a carrier or as a component in a fusion polypeptide, for example) or a polypeptide fragment derived from that protein or a related amino acid sequence that is capable of eliciting a desired biological effect, such as catalysing a particular biochemical reaction, eliciting an immune response against that protein or of being recognised by an antibody specific to that protein, of mediating a cell-signalling effect, or the like. Such a polypeptide fragment may be referred to with reference to the function it possesses, such as the function it shares with the full-length protein from which it was derived. For example, a polypeptide fragment having an immunological effect may be referred to as an immunogenic fragment, where an “immunogenic fragment” is understood to be a fragment of the full-length protein that retains its capability to induce an immune response in a vertebrate host or be recognised by an antibody specific to the parent protein. Similarly, a polypeptide fragment retaining or possessing one or more biological effects elicited by the full-length protein from which it was derived, or possessing a related or different biological effect, is referred to herein as a “bioactive fragment” or a “bioactive polypeptide fragment”. Likewise, a polypeptide having a biological effect, such as a polypeptide capable of stimulating a biological response in a cell or eliciting a therapeutic effect, may be referred to herein as a “bioactive fragment” or a “bioactive polypeptide fragment”, or grammatical equivalents thereof.

A variety of techniques is available to identify such polypeptide fragments, as well as DNA fragments encoding such fragments. For example, in the case of immunogenic fragments, such fragments may comprise one or more determinants or epitopes. Well-established empirical and in silico methods for the detection of epitopes exist and are well known to those skilled in the art. For example, computer algorithms are able to designate specific protein fragments as the immunologically important epitopes on the basis of their sequential and/or structural agreement with epitopes that are known. The determination of these regions is typically based on a combination of the hydrophilicity criteria and secondary structural features. An immunogenic fragment (or epitope) usually has a minimal length of 6, more commonly 8 amino acids, preferably more then 8, such as 9, 10, 12, 15 or even 20 or more amino acids. The nucleic acid sequences encoding such a fragment therefore have a length of at least 18, more commonly 24 and preferably 27, 30, 36, 45 or even 60 nucleic acids.

Similarly, those skilled in the art will be aware of methods to identify bioactive fragments using various assays targeted at identifying or detecting a particular biological response. Representative methods suitable for use in the identification or detection of bioactive fragments contemplated herein are presented below, including in the Examples.

The term “variant” with reference to polypeptides encompasses naturally occurring, recombinantly, and synthetically produced polypeptides, including those comprising one or more non-natural amino acids, one or more amino acid analogues, and peptide mimetics. Variant polypeptide sequences preferably exhibit at least 50%, more preferably at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least %, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a sequences of the present invention. Identity is found over a comparison window of typically at least 20 amino acid positions, preferably at least 50 amino acid positions, at least 100 amino acid positions, or over the entire length of a polypeptide of the invention.

Polypeptide sequence identity can be determined in the following manner. The subject polypeptide sequence is compared to a candidate polypeptide sequence using BLASTP (from the BLAST suite of programs, version 2.2.10 [October 2004]) in bl2seq, which is publicly available from NCBI (ftp://ftp.ncbi.nih.gov/blast/). The default parameters of bl2seq are utilized except that filtering of low complexity regions should be turned off.

Polypeptide sequence identity may also be calculated over the entire length of the overlap between a candidate and subject polynucleotide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

Polypeptide variants contemplated herein also encompass those which exhibit a similarity to one or more of the specifically identified sequences that is likely to preserve the functional equivalence of those sequences and which could not reasonably be expected to have occurred by random chance. Such sequence similarity with respect to polypeptides can be determined using the publicly available bl2seq program from the BLAST suite of programs (version 2.2.10 [October 2004]) from NCBI (ftp://ftp.ncbi.nih.gov/blast/). The similarity of polypeptide sequences can be examined using the following unix command line parameters:

bl2seq-i peptideseq1-j peptideseq2-F F-p blastp

Variant polypeptide sequences preferably exhibit an E value of less than 1×10⁻¹⁰, more preferably less than 1×10⁻²⁰, less than 1×10⁻³⁰, less than 1×10⁻⁴⁰, less than 1×10⁻⁵⁰, less than 1×10⁻⁶⁰, less than 1×10⁻⁷⁰, less than 1×10⁻⁸⁰, less than 1×10⁻⁹⁰, less than 1×10⁻¹⁰⁰, less than 1×10⁻¹¹⁰ than 1×10⁻¹²⁰ or less than 1×10⁻¹²³ when compared with any one of the specifically identified sequences.

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Conservative substitutions of one or several amino acids of a described polypeptide sequence without significantly altering its biological activity are also included in the invention. A skilled artisan will be aware of methods for making phenotypically silent amino acid substitutions (see, e.g., Bowie et al., 1990, Science 247, 1306).

A polypeptide variant contemplated herein also encompasses that which is produced from the nucleic acid encoding a polypeptide, but differs from the wild type polypeptide in that it is processed differently such that it has an altered amino acid sequence. For example, in one embodiment a variant is produced by an alternative splicing pattern of the primary RNA transcript to that which produces a wild type polypeptide.

Host Cells

Those skilled in the art will recognise on reading this description that the desired complement of genes and/or polynucleotides will in certain embodiments be present in one or multiple constructs that are transformed into a host cell. Similarly, in certain embodiments, the one or more genes, polynucleotides, or constructs as contemplated herein are stably incorporated into the genome of a host cell.

It will also be appreciated, having regard to the discussion herein, that a wide variety of host cells are amenable to use as contemplated herein. In certain embodiments, the host cell is from a fungal species. In one example, the host cell is a fungal cell other than a yeast cell. In another example, the host cell is a yeast cell.

In a further embodiment the host cell is from a bacterial species.

In one embodiment the host cell is from the subkingdom Dikarya.

In various embodiments the host cell is from a phylum selected from Chytridiomycota, Neocallimastigomycota, Blastocladiomycota, Glomeromycota, Ascomycota and Basidiomycota or a subphylum incertae sedis selected from Mucoromycotina, Kickxellomycotina, Zoopagomycotina and Entomophthoromycotina.

In one embodiment the host cell is from an order selected from Mucorales, Hypocreales, Eurotiales, Sebacinales and Saccharomycetales.

In certain examples, the host cell is from a genus selected from Metarhizium, Epichloë, Saccharomyces, Kluveromyces, Trichoderma, Aspergillus, Beauveria, Pichia, Penicillium, Serendipita, Umbelopsis, Neurospora, Epicoccum, Sarocladium, Balansia, Fusarium, Alternaria, Ustilago, Sebacina, Glomus and Rhizopus.

In various embodiments the host cell is from a species selected from the group comprising Metarhizium robertsii; Trichoderma reesei; Aspergillus niger; Aspergillus nidulans; Aspergillus oryzae; Beauveria bassiana; Saccharomyces cerevisiae; Pichia pastoris; Kluveromyces marxianus; Epichloë festucae; Epichloë typhina; Penicllium chrysogenum; Penicillium paxilli; Penicillium expansum; Serendipita indica; Umbelopsis isabellina; Neurospora crassa; Epicoccum italicum; Sarocladium zeae; Fusarium verticillioides; Ustilago maydis.

In one embodiment the host cell is from species or strain, for example from a species or strain of fungi, that is amenable to culture, including liquid or solid phase culture, and/or is amenable to use in fermentation. In various examples, the host cell from a species or strain amenable to culture, and/or amenable to fermentation, is from a genus selected from: Aspergillus, Beauveria, Epichloë, Neurospora, Epicoccum, Sarocladium, Kluveromyces, Metarhizium, Penicillium, Pichia, Rhizopus, Saccharomyces, Serendipita, Trichoderma, and Umbelopsis.

In a one example, the host cell from a species or strain amenable to culture, and/or amenable to fermentation is from a species selected from: Aspergillus niger, Aspergillus nidulans, Aspergillus oryzae, Beauveria bassiana, Epichloë festucae, Epichloë typhina, Epicoccum italicum, Metarhizium robertsii, Penicillium expansum, Penicillium chrysogenum, Penicillium paxilli, Saccharomyces cerevisiae, Kluveromyces marxianus, Pichia pastorus, Rhizopus oryzae, Rhizopus stolonifer, Rhizopus microsporus, Serendipita indica, Trichoderma reesei, Neurospora crassa, Sarocladium zeae and Umbelopsis isabellina.

In various embodiments, the host cell is an endophytic cell, such as an endophytic cell capable of forming a stable symbiont with a plant or plant cell.

Methods for Producing a Host Cell

In one aspect the invention relates to a method for producing a host cell capable of producing at least one epoxy-janthitrem compound, the method comprising modifying or transforming a host cell to comprise at least one polynucleotide as herein described, or to express one or more polypeptides as herein described.

In one embodiment the host cell is produced by modifying or transforming a cell to comprise at least one polynucleotide or construct as herein described.

In various embodiments, the host cell is modified or transformed to comprise at least one, for example two, or for example three polynucleotides selected from the group comprising SEQ ID NO: 1, 2, 4, 5, 7, 8, 10 to 16, 18, 20, and 22. In other embodiments, the host cell is modified or transformed to comprise at least one, for example two or more, for example three or more, four or more, or five or more polynucleotides selected from the group comprising SEQ ID NO: 1, 2, 4, 5, 7, 8, 16, 18, 20, and 22, and comprises at least one or more, for example at least two, at least three, at least 4, at least 5, at least 6, for example at least 7, or at least 8 of the polynucleotides selected from the group comprising: a polynucleotide encoding the gene idtG, a polynucleotide encoding the gene idtM, a polynucleotide encoding the gene idtB, a polynucleotide encoding the gene idtC, a polynucleotide encoding the gene idtP, a polynucleotide encoding the gene idtQ, a polynucleotide encoding the gene idtF, and a polynucleotide encoding the gene idtK.

In one embodiment, the host cell is modified or transformed to comprise at least one polynucleotide selected from the group comprising SEQ ID NO: 1 and 2, at least one polynucleotide selected from the group comprising SEQ ID NO: 4 and 5, and at least one polynucleotide selected from the group comprising SEQ ID NO: 7 and 8. In one embodiment, the host cell comprises at least one gene from the group comprising idtG, idtM, idtB, idtC, idtP, idtQ, idtF, and idtK.

In various embodiments, the host cell comprises each of the genes from the group comprising idtG, idtM, idtB, idtC, idtP, and idtQ. In one example, the host cell additionally comprises the gene idtF. In another example, the host cell additionally comprises the gene idtK. In a further example, the host cell additionally comprises the gene idtF and the gene idtK.

In various embodiments, the host cell is modified or transformed to comprise at least one, for example at least two, for example at least three, at least four, or at least five polynucleotides selected from the group comprising SEQ ID NO: 1, 2, 4, 5, 7, 8, 10 to 16, 18, 20, and 22, and the host cell comprises at least one or more, for example at least two, at least three, at least 4, at least 5, or at least 6 of the polynucleotides selected from the group comprising: a polynucleotide encoding the gene idtG, a polynucleotide encoding the gene idtM, a polynucleotide encoding the gene idtB, a polynucleotide encoding the gene idtC, a polynucleotide encoding the gene idtP, and a polynucleotide encoding the gene idtQ.

In various embodiments, the host cell is modified or transformed to comprise at least one, for example at least two, for example at least three or more polynucleotides selected from the group comprising SEQ ID NO: 1, 2, 4, 5, 7, 8, 10 to 15, and is modified or transformed to comprise at least one, for example two or more polynucleotides selected from the group comprising SEQ ID NO: 16, 18, 20, and 22, and the host cell comprises at least one or more, for example at least two, at least three, at least 4, at least 5, or at least 6 of the polynucleotides selected from the group comprising: a polynucleotide encoding the gene idtG, a polynucleotide encoding the gene idtM, a polynucleotide encoding the gene idtB, a polynucleotide encoding the gene idtC, a polynucleotide encoding the gene idtP, and a polynucleotide encoding the gene idtQ.

In one embodiment, the host cell is modified or transformed to comprise at least one polynucleotide selected from the group comprising SEQ ID NO: 1 and 2, at least one polynucleotide selected from the group comprising SEQ ID NO: 4 and 5, at least one polynucleotide selected from the group comprising SEQ ID NO: 7 and 8, at least one polynucleotide from the group comprising SEQ ID NO: 16 and 18, and at least one polynucleotide from the group comprising SEQ ID NO: 20 and 22. In one embodiment, the host cell comprises at least one gene from the group comprising idtG, idtM, idtB, idtC, idtP, idtQ. In one embodiment, the host cell comprises each of the genes from the group comprising idtG, idtM, idtB, idtC, idtP, and idtQ.

In various embodiments, the host cell is modified or transformed to comprise at least one polynucleotide selected from the group comprising: a polynucleotide encoding the gene idtG, a polynucleotide encoding the gene idtM, a polynucleotide encoding the gene idtB, a polynucleotide encoding the gene idtC, a polynucleotide encoding the gene idtP, and a polynucleotide encoding the gene idtQ.

The host cells described herein comprise a genome encoding, capable of expressing, and/or having been modified or transformed with one or more polynucleotides as described herein, such as one or more genes involved in the epoxy-janthitrem biosynthetic pathway, or one or more expression constructs comprising same.

Accordingly, in one embodiment the invention relates to a host cell that expresses at least one heterologous polypeptide that catalyzes the conversion of a substrate in the epoxy-janthitrem biosynthetic pathway, for example, in the epoxy-janthitrem biosynthetic pathway leading to the formation of any one or more of epoxy-janthitrem I, epoxy-janthitrem II, epoxy-janthitrem III, epoxy-janthitrem IV, or epoxy-janthitriol.

In various embodiments, the substrate is selected from the group consisting of isopentyl pyrophosphate, farnesyl pyrophosphate, and indole-3-glycerol phosphate.

In various embodiments, the substrate is selected from the group comprising terpendole C, terpendole J, terpendole I, terpendole B, terpendole G, terpendole F, terpendole E, α-paxitriol, α-PC-M6, paspaline B, intermediate 1, paspaline, and emindole SB, or any combination thereof.

In various embodiments, the at least one heterologous polypeptide catalyzes one of the conversions selected from the group comprising:

a. the conversion of terpendole Ito epoxy-janthitriol;

b. the conversion of terpendole J to epoxy-janthitrem III;

c. the conversion of terpendole C to epoxy-janthitrem II;

d. the conversion of epoxy-janthitriol to epoxy-janthitrem I;

e. the conversion of epoxy-janthitrem III to epoxy-janthitrem IV; and

f. any combination of two or more of a) to e) above.

In various embodiments, the conversion is a prenylation, a cyclisation, an acylation, a condensation, or an oxidation. For example, the conversion is a prenylation, a cyclisation, or an acylation.

In various embodiments the expression construct comprises two or more, or three or more genes involved in the epoxy-janthitrem biosynthetic pathway.

In various embodiments the one or more genes involved in the epoxy-janthitrem biosynthetic pathway are operably linked to one or more regulatory elements that control the transcription, translation or expression of the one or more genes in the host cell into which it is introduced. The one or more regulatory elements may be contiguous with the one or more genes involved in the epoxy-janthitrem biosynthetic pathway, or act in trans or at a distance to control the gene of interest.

Suitable regulatory elements include appropriate transcription initiation, termination, promoter and enhancer sequences, or RNA processing signals such as splicing or polyadenylation signals.

Examples of suitable promoters for use in fungal host cells include promoters which are homologous or heterologous to the host cell. Furthermore, suitable promoters for use in the expression constructs contemplated herein include constitutive promoters, regulatable promoters, inducible promoters or repressible promoters. The promoter is in certain embodiments derived from a gene of the host cell, or is a promoter derived from the genes of other species, such as other fungi, viruses or bacteria. Those skilled in the art will, without undue experimentation, be able to select promoters that are suitable for use in modifying and modulating expression constructs using genetic constructs comprising the genes involved in the epoxy-janthitrem biosynthetic pathway of the sequences described herein.

In certain embodiments where the polynucleotide(s) or expression construct(s) comprise two or more genes involved in the epoxy-janthitrem biosynthetic pathway, or where the host cell comprises or has been modified or transformed with two or more genes involved in the epoxy-janthitrem biosynthetic pathway or to express one or more polypeptides as described herein, each gene is under the control of the same promoter, while in other embodiments, each gene is under the control of different promoters.

In various embodiments the method comprises transforming the host cell with one or more, two or more, or three or more expression constructs as contemplated herein.

Host cells may be transformed using suitable methods known in the art for achieving heterologous gene expression in fungi and/or yeast. Choice of transformation method will depend on the species and form of the host cell, and the number of expression constructs and or LOL genes to be transformed.

In one embodiment the method comprises transforming the host cell with a polynucleotide, vector or construct so that the one or more genes involved in the epoxy-janthitrem biosynthetic pathway is integrated into the genome of the host cell via homologous or non-homologous recombination.

Methods to prepare host cells, such as fungal host cells, for transformation are well known in the art. In various embodiments the host cell comprises protoplasts, spheroplasts, spores or conidia. A representative method of producing a fungal host cell, involving the preparation of protoplasts and their subsequent transformation with both linear DNA encoding the gene(s) of interest and plasmid-borne selectable markers is described herein in the Examples.

In one embodiment the method comprises transforming the host cell using polyethylene glycol (PEG)-mediated transformation. Other suitable transformation methods include electroporation, Agrobacterium tumefaciens-mediated transformation, biolistic transformation, or non-PEG-mediated spheroplast transformation.

A further exemplary method that may be used to achieve homologous recombination of one or more genes involved in the epoxy-janthitrem biosynthetic pathway into a fungal host cell genome using sequential transformations is that described by Chiang and co-workers (Chiang, et al., 2013).

For genes that are very large and difficult to amplify by PCR, two smaller transforming fragments are created that fuse by homologous recombination in vivo to reconstruct the full-length coding sequences under the control of a single promoter. In certain exemplary methods contemplated and exemplified herein, two or more genes involved in the epoxy-janthitrem biosynthetic pathway are integrated into the host cell genome using sequential transformations. In certain embodiments, each gene or transforming fragment carries a selectable marker to enable selection of successful transformants.

Gene Editing/CRISPR

In other embodiments, the host cell genome has been modified, for example by a gene editing method, to render the host cell capable of producing one or more epoxy-janthitrem compounds.

Endonuclease-based systems, also referred to as Sequence-specific nuclease (SSN) systems, have rapidly become the principal gene editing tools used in molecular biology, amenable for use in gene disruption and gene targeting, in addition to targeted incorporation of new genetic material into host genomes. Endonuclease-based systems for gene editing allow the modification of genomes with high precision, efficiency, and flexibility. Examples of endonuclease-based approaches for gene editing include systems comprising, without limitations, zinc finger nucleases (ZFNs), TAL effector nucleases (TALENs), meganucleases (such as MegaTALs), and CRISPR/Cas9 and related systems.

In one embodiment, the generation of a host cell capable of producing one or more epoxy-janthitrem compounds is by targeted genome modification comprising the use of SSNs. In one example, the SSNs are selected from ZFNs, TALENs, or CRISPR/Cas. In one embodiment, the SSN is selected from a TALEN. In another embodiment, the SSN is selected from CRISPR/Cas. Particularly contemplated gene editing systems for the production of a modified host cell capable of producing one or more epoxy-janthitrem compounds are CRISPR/Cas9 and its various derivatives, including CRISPR systems utilising modified Cas9 proteins, functional fragments thereof, and/or homologues or variants thereof. Representative examples of CRISPR/Cas9 gene editing methods are presented herein in the Examples.

Production of Epoxy-Janthitrems

In a further aspect the invention relates to a method for producing at least one epoxy-janthitrem compound, the method comprising culturing one or more host cells as described herein, such as one or more host cells produced by a method as herein described, under conditions conducive to the production of the at least one epoxy-janthitrem compound, by the host cells. In one embodiment the method further comprises separating, purifying, fractionating, or isolating the at least epoxy-janthitrem compound.

It will be appreciated that the use of one or more host cells, or a population of host cells together with one or more other cells, that is tolerant of one or more epoxy-janthitrem compounds and/or precursors thereof is advantageous to, for example, in vitro and/or ex-planta production of one or more epoxy-janthitrem compounds. In this context, ex-planta production contemplates the production of one or more epoxy-janthitrem compounds outside of a whole plant. Accordingly, ex-planta production includes production of one or more epoxy-janthitrem compounds without the use of any plant cells, or production using one or more plant cells, for example in a co-culture with one or more other cell types, such as one or more endophytic fungal cells. Particularly contemplated ex-planta production comprises the use of a population of host cells as contemplated herein, such as one or more modified or transformed Epichloë cells, optionally together with a population of plant cells, for example in a liquid or solid-phase co-culture. Accordingly, ex-planta production encompasses in vitro production methods.

Host cells particularly contemplated for use in ex-planta and/or in vitro production methods include tractable fungi amenable to growth in culture, including growth at large scale, such as in bioreactors, large culture vessels, continuous phase culturing, and the like. Representative tractable fungi include Penicillium spp., including for example Penicillium paxilli, Penicillium janthinellum, and Penicillium shearii.

In various embodiments the host cells are cultured in the presence of at least one epoxy-janthitrem compound precursor.

For example, in various embodiments the method comprises maintaining the host cells in the presence of at least one of:

a) an effective amount of farnesyl pyrophosphate or a biosynthetic precursor thereof,

b) an effective amount of isopentyl pyrophosphate or a biosynthetic precursor thereof,

c) an effective amount of indole-3-glycerol phosphate or a biosynthetic precursor thereof,

d) an effective amount of emindole SB or a biosynthetic precursor thereof,

e) an effective amount of terpendole I or a biosynthetic precursor thereof, or

f) a compound of any one of formulae IV to VIII, or

g) any combination of two or more of (a) to (f) above.

In one embodiment the method comprises maintaining the one or more host cells, for example a culture thereof, at a temperature of from about 15 ° C. to about 35 ° C. In a further embodiment the method comprises maintaining the one or more host cells, for example a culture thereof, at a temperature of from about 20 ° C. to about 40 ° C.

In one embodiment the method comprises maintaining the host cells, or a culture thereof, at for at least about 1 day, at least about 3 days, at least about 4 days, at least about 7 days or at least about 10 days.

In one embodiment the purification or isolation is achieved via filtration and/or column purification.

It will be appreciated from this disclosure that in one aspect the invention relates to a method for conferring on an organism the ability to produce one or more epoxy-janthitrem compounds, the method comprising transforming a host cell comprising the organism with one or more genes involved in the epoxy-janthitrem biosynthetic pathway, such as with an expression construct as herein described.

In one embodiment the organism, prior to transformation, does not produce the epoxy-janthitrem compound. In one example, the organism is capable of producing one or more epoxy-janthitrem precurors.

The cell as herein described may be part of an organism. Thus reference to a cell or host cell can be used interchangeably with reference to an organism or host organism.

In Planta Production

It will be appreciated from this disclosure that in one aspect the invention relates to a method for conferring on an organism the ability to produce one or more epoxy-janthitrem compounds, the method comprising providing the organism with a host cell modified or transformed with or to comprise one or more genes involved in the epoxy-janthitrem biosynthetic pathway, such as one or more polynucleotides encoding a polypeptide as herein described, such as with an expression construct as herein described.

In one particularly contemplated embodiment, the organism is a plant and the host cell is an endophytic fungal cell. For example, the organism is a plant, such as a rye grass, and the endophytic fungal cell is an Epichloë cell.

Representative methods for conferring on a plant the ability to produce one or more epoxy-janthitrem compounds by inoculating the plant with an endophytic fungi are presented herein in the examples. Such methods are amenable to adaptation to other plant/fungal or plant/host cell systems, including systems in which one or more of the plant cells are modified or transformed with or to comprise one or more genes involved in the epoxy-janthitrem biosynthetic pathway, such as one or more polynucleotides encoding a polypeptide as herein described.

Agricultural Compositions

While the in-planta production of epoxy-janthitrem compounds by plant/endophytic fungi symbionts represents one particularly contemplated mode of applying the invention, the use of host cells as described herein and/or epoxy-janthitrem compounds produced by host cells as described herein, in other applications of this invention is also contemplated—including, for example, in the formulation of a composition for agricultural application.

In certain embodiments, compositions for agricultural application, such as in the control of one or more plant pests, will typically include in addition to the one or more host cells and/or epoxy-janthitrem compounds at least one agriculturally-acceptable carrier, such as one or more humectants, spreaders, stickers, stabilisers, penetrants, emulsifiers, dispersants, surfactants, buffers, binders, protectants, and other components typically employed in agricultural compositions, or in insecticidal or pesitcidal compositions.

Compositions contemplated herein may be formulated in a variety of different ways without departing from the scope of the present invention. The composition of the invention may be in liquid or solid form. In general, the formulation chosen will be dependent on the end application. For example, possible formulations include, but should not be limited to matrixes, soluble powders, granules including water dispersible granules, encapsulations including micro-encapsulations, aqueous solutions, aqueous suspensions, non-aqueous solutions, non-aqueous suspensions, emulsions including microemulsions, pastes, emulsifiable concentrations, and baits.

In various embodiments, the agricultural composition is a liquid composition. Liquid compositions typically include water, saline or oils such as vegetable or mineral oils. Examples of vegetable oils useful in the invention are soybean oil and coconut oil. The compositions may be in the form of sprays, suspensions, concentrates, foams, drenches, slurries, injectables, gels, dips, pastes and the like. Conventional formulation techniques suitable for the production of liquid compositions are well known in the art.

In various embodiments the composition is in solid form. For example, solid inorganic agricultural carriers suitable for use include carbonates, sulphates, phosphates or silicates, pumice, lime, bentonite, or mixtures thereof. Solid biological materials suitable for use include powdered palm husks, corncob hulls, and nut shells.

Exemplary solid agricultural compositions include those formulated as dusts, granules incluing water dispersible granules, seed coatings, wettable powders or the like. As is understood in the art, certain solid compositions are applied in solid form, while others are formulated to be admixed with a liquid prior to application, so as to provide a liquid agricultural composition for application.

The compositions contemplated herein are in certain embodiments in the form of controlled release, or sustained release formulations. The compositions contemplated herein in certain embodiments also include other control agents such as pesticides, insecticides, fungicides, nematocides, virucides, growth promoters, nutrients, germination promoters and the like, provided they are compatible with the activity of one or more epoxy-janthitrem compounds produced in accordance with the description herein, for example, one or more epoxy-janthitrem compounds produced by or using a host cell, expression construct, polynucleotide, or polypeptide as herein described, or the host cell or related agent.

The invention is further described with reference to the following examples. It will be appreciated that the invention as claimed is not intended to be limited in any way by these examples.

### EXAMPLES

### Example 1

This example describes the generation of a heterologous host capable of producing for the first time one or more epoxy-janthitrem compounds, and the characterisation of epoxy-janthitrem compound production in said host.

Methods

Experiments were performed with Epichloë festucae var. lolii strain AR6 and AR37. Fungal strains were grown on PDA (Oxoid) at 22° C. and supplemented where necessary with hygromycin (150 μg/ml) and geneticin (250 μg/mI)

Preparation of the Insertion Construct

Fungal genomic DNA was isolated from mycelium using a Zymo Fungal/Bacterial DNA mini prep kit. Plasmid DNA was isolated and purified using an Invitrogen plasmid mini prep kit.

The linear PCR products used for transformation were amplified using PrimeSTAR polymerase (Takara) from AR37 genomic DNA using the PCR primers shown in Table 1 below.

TABLE 1
PCR primers and products

| Primer | Sequence | SEQ ID NO: | Product size |
|---|---|---|---|
| idtA 95889F | CTTATAACTATATTAGGGGAGC | 10 | 2315 bp |
| idtA 98203R | GTGTGTCTCTATGGGGGTTTG | 11 | |
| idtD MG159 | AGGAAGTCACGCAGGAGTGGG | 12 | 2790 bp |
| idtD MG158 | AAGGCAGTTTTGCAAGGAATGTCTT | 13 | |
| idtO 48763F | GGCGCGCACTTAATAGGCTTAC | 14 | 3134 bp |
| idtO 51896R | GGAGAATCTGACGGAAAGGAG | 15 | |

One PCR product for each of the AR37 genes idtA, idtD, and idtO was synthesized, each including the target gene plus ˜1 kb of upstream (promoter) sequence and ˜200 bp of downstream (terminator) sequence. The sequence of the idtA PCR product is presented herein as SEQ ID NO: 1, that of the idtD PCR product is presented herein as SEQ ID NO: 4, and the sequence of the idtO PCR product is presented herein as SEQ ID NO: 7. The idtA, idtD, and idtO coding sequences comprised with these PCR products are presented herein as SEQ ID NO: 2, SEQ ID NO: 5, and SEQ ID NO: 8, respectively. PCR products were gel extracted (Invitrogen PureLink Gel Extraction kit), then cleaned and concentrated (Zymo clean and concentrator kit).

Fungal protoplast. Protoplasts of E. festucae var. lolii AR6 were prepared using modifications of the method of Young et al., 1998. Fungal cultures were grown in 50 ml defined media recipe from Kulkarni and Nielsen (Kulkarni and Nielsen, 1986) inoculated with macerated fungi (grown on cellophane PDA plates for 7 days at 22 ° C.) and grown at 22 ° C. with moderate shaking (150 rpm) for 5 days. The mycelial pellets were washed with 1 L sterile H₂0 followed by a wash in OM buffer (1.2 M MgSO₄, 10 mM Na₂HPO₄, pH 5.8). The washed mycelia were mixed with 30 ml of sterile OM buffer containing 15 mg/ml Trichoderma lysing enzyme (Sigma). This mixture was shaken gently (100 rpm) for 18 hours at 30 ° C. The digested hyphae were filtered through Mira-cloth (Calbiochem) and the filtrate, containing the protoplasts, was over-laid with 2 ml STC buffer (0.6 M sorbitol, 100 mM Tris-HCL pH 8.0). The protoplasts were banded at the interface by centrifugation at 3000 g for 15 min, washed three times with 10 ml STC buffer (1 M sorbitol, 50 mM CaCl₂, 50 mM Tris/HCl pH 8.0) by centrifuging at 7700 g and resuspended into STC buffer to a final concentration of 1.25×10⁸ per ml.

Transformation of E. festucae var. lolli Protoplasts and Molecular Analysis of Transformants.

Protoplasts prepared as described above were transformed with 500 fmol linear DNA PCR product and co-transformed with 800 ng of plasmid containing either the hygromycin or geneticin selectable makers. The following combinations of PCR products were transformed into AR6: idtD, idtO and idtA individually; idtD and idt0 together; all three genes idtD, idtO and idtA. Transformants were selected on RG Media (PD with 0.8 M sucrose pH 6.5) containing hygromycin (150 μg/ml) and/or geneticin (250 μg/ml). To obtain clonal isolates, the resulting transformants were purified by sub-culturing three times as described by Young et al., 2005.

Transformants were screened by PCR for their random integration into AR6 with the primers used to make the insertion PCR products.

Inoculation into Ryegrass and Immunoblotting

Inoculation of endophyte-free seedlings of perennial ryegrass (Lolium perenne cv. Nui) was carried out using the method of Latch and Christensen (Latch and Christensen, 1985) with mycelium from 5- to 14-day-old cultures. Plants were tested for endophyte infection by tissue print immunoblotting (Gwinn et al., 1991).

Chemistry Methods

Sample Preparation

Basal sections of leaf blades were harvested from tillers of each plant. Samples were harvested into liquid nitrogen and then transferred to a freeze-drier (Freezone Plus12, Labconco Corporation, Kansas City, Mich., USA). Once lyophilized the samples were ground and homogenized with a bead mill

(Omni Bead Ruptor 24, Omni International Inc., Kennesaw, Ga., USA) in a 7 mL vial using a ¼ inch zirconium bead (30 seconds at 4.5 m/s).

For analysis, sub-samples (50 mg) were extracted with 1 mL of the prepared extraction solvent (80% v/v methanol with 0.54 ng/mL ergotamine, 0.202 ng/ml festuclavine, and 1.7 ng/mL homoperamine as internal standards) in 2 mL plastic vials for 1 hour by end-over-end rotation (30 Hz) in the dark. After centrifuging (5000 g, 5 min), the supernatant was transferred to 2 mL amber HPLC vials for analysis.

Along with the samples, duplicate reference samples of AR37 (for quantifying the epoxy-janthitrems) were similarly extracted (with 80% acetone substituted for the 80% methanol) and analysed. Due to the instability of the epoxy-janthitrems as pure compounds, they are not available as standards. Therefore in place of pure standards, the concentrations of the five epoxy-janthitrem compounds has been determined in the AR37 reference material, and then used to generate the response factors for quantitation.

Analysis of Epichloë Indole Diterpenes (QQQ MRM Method)

Samples (5 μL injection) were chromatographically separated on a Kinetic C18 150×2.1 mm (2.6 μm) column (Phenomenex, Torrance, Calif., USA) using the following linear gradient profile (eluent A, aqueous 0.1% formic acid and eluent B, acetonitrile with 0.1% formic acid); time 0 min (T₀) at 10% B, T₅ at 60% B, T₁₇ at 100% B, and T₁₉ at 100% B, followed by equilibration to initial conditions over the following 8 min. Detection and quantitation was achieved using a QTRAP 6500+ (AB Sciex LLC, Framingham, Mass., USA) using ESI in positive ion mode and a nebulizer temperature of 400° C. Table 2 shows the parameters specific to each compound.

TABLE 2
Mass spectrometer parameters for individual compounds.

| Compound | R.T. | MRM | DP | CE | CXP | IS | CUR | GS1 | GS2 |
|---|---|---|---|---|---|---|---|---|---|
| Paspaline | 14.54 | 422.3/130.1 | 100 | 50 | 30 | 3500 | 30 | 30 | 40 |
| | | 422.3/404.2 | 40 | 20 | 20 | 3000 | 50 | 70 | 30 |
| Terpendole E | 9.76 | 438.2/130.1 | 20 | 20 | 20 | 5000 | 40 | 40 | 50 |
| | | 438.2/423.2 | 60 | 10 | 10 | 4000 | 30 | 70 | 40 |
| Paspaline B | 9.37 | 436.3/130.1 | 30 | 20 | 50 | 4500 | 50 | 70 | 30 |
| | | 436.3/418.2 | 30 | 20 | 10 | 4000 | 30 | 70 | 70 |
| Terpendole I | 7.97 | 454.3/130.1 | 30 | 50 | 30 | 5000 | 40 | 30 | 70 |
| | | 454.3/436.2 | 90 | 20 | 50 | 5500 | 20 | 50 | 50 |
| Terpendole J | 12.95 | 522.3/130.1 | 50 | 30 | 40 | 4500 | 20 | 40 | 40 |
| | | 522.3/507.3 | 50 | 10 | 30 | 5500 | 30 | 30 | 50 |
| Terpendole C | 12.67 | 520.3/130.1 | 90 | 10 | 40 | 3000 | 40 | 40 | 30 |
| | | 520.3/505.3 | 80 | 10 | 30 | 2500 | 50 | 50 | 70 |
| Terpendole K | 12.33 | 518.3/130.1 | 20 | 20 | 20 | 4500 | 20 | 30 | 50 |
| | | 518.3/503.3 | 0 | 10 | 20 | 4500 | 40 | 70 | 70 |
| Terpendole N | 11.12 | 534.3/130.1 | 90 | 20 | 55 | 5000 | 50 | 50 | 60 |
| | | 534.3/516.2 | 20 | 20 | 30 | 5500 | 20 | 50 | 60 |
| Epoxy-Janthitriol | 11.25 | 604.4/222.1 | 80 | 50 | 10 | 4500 | 30 | 50 | 40 |
| | | 604.4/546.1 | 20 | 10 | 10 | 3500 | 40 | 50 | 50 |
| Epoxy-janthitrem I | 12.70 | 646.4/222.1 | 10 | 50 | 10 | 4000 | 20 | 70 | 70 |
| | | 646.4/588.1 | 70 | 30 | 50 | 5500 | 30 | 40 | 70 |
| Epoxy-janthitrem II | 16.51 | 670.4/222.1 | 10 | 40 | 10 | 3000 | 20 | 50 | 30 |
| | | 670.4/612.1 | 70 | 40 | 10 | 2500 | 30 | 50 | 40 |
| Epoxy-janthitrem III | 16.87 | 672.4/222.1 | 30 | 30 | 10 | 2500 | 20 | 30 | 70 |
| | | 672.4/614.1 | 0 | 30 | 50 | 5000 | 50 | 30 | 70 |
| Epoxy-janthitrem IV | 17.03 | 714.4/222.1 | 60 | 50 | 40 | 3000 | 30 | 70 | 60 |
| | | 714.4/656.1 | 60 | 30 | 55 | 3500 | 30 | 30 | 40 |

Data Processing

The raw data was processed using MultiQuant v3.0.2 (AB Sciex LLC, Framingham, Mass., USA) to integrate and determine peak areas for target compounds. For the non-epoxy-janthitrem compounds, quantitation was achieved by comparison of the peak areas to a paxilline standard (220 ng/mL) and are reported as paxilline standard equivalent (pg/g DM). For the epoxy-janthitrems, the peak areas and known concentrations of the reference samples (AR37) were used to determine a response factor, which was used to subsequently quantify each epoxy-janthitrem compound in the samples.

Results

The following tables (Table 3 & Table 4) show the results of the two sets of samples that were analysed.

TABLE 3
LC-MS/MS analysis results for first sample set showing detection
and quantification of selected indole diterpenes.

| | Endophyte | | | | | | |
|---|---|---|---|---|---|---|---|
| | AR37 | AR6 | | | | | |
| | Construct | | | | | | |
| | | | | | | | idtD |
| | wild- | wild- | | | | idtD | idtO |
| | type | type | idtA | idtD | idtO | idtO | idtA |
| Paspaline ^{#} | 0.08 | 1.21 | 2.00 | 2.53 | 1.27 | 1.48 | 1.31 |
| Terpendole E ^{#} | 0.02 | 0.09 | 0.14 | 0.25 | 0.11 | 0.11 | 0.07 |
| Paspaline B ^{#} | 0.02 | 0.06 | 0.18 | 0.10 | 0.06 | 0.11 | 0.13 |
| Terpendole I ^{#} | 0 | 1.17 | 1.57 | 0.82 | 1.22 | 0.48 | 0.38 |
| Terpendole J ^{#} | 0.01 | 0.07 | 0.10 | 0.27 | 0.07 | 0.07 | 0.04 |
| Terpendole C ^{#} | 0.00 | 0.05 | 0.04 | 0.04 | 0.04 | 0.01 | 0.01 |
| Terpendole K ^{#} | 0.00 | 0.25 | 0.60 | 0.39 | 0.34 | 0.14 | 0.09 |
| Terpendole N ^{#} | 0 | 1.41 | 1.98 | 0.87 | 1.47 | 0.58 | 0.55 |
| Epoxy-Janthitriol ^{‡} | 9.7 | 0 | 0 | 0 | 0 | 22.0 | 9.7 |
| Epoxy-janthitrem I ^{‡} | 46.9 | 0 | 0 | 0 | 0 | 0.3 | 18.4 |
| Epoxy-janthitrem II ^{‡} | 25.8 | 0 | 0 | 0 | 0 | 4.8 | 2.8 |
| Epoxy-janthitrem III ‡ | 28.1 | 0 | 0 | 0 | 0 | 9.7 | 9.2 |
| Epoxy-janthitrem IV ^{‡} | 11.7 | 0 | 0 | 0 | 0 | 0 | 1.5 |

^{#} Values are paxilline standard equivalent (μg/g DM)
^{‡} Values are ug/g DM

TABLE 4
LC-MS/MS analysis results for second sample set showing detection
and quantification of selected indole diterpenes.

| | Endophyte | | | | |
|---|---|---|---|---|---|
| | AR37 | AR6 | | | |
| | Construct | | | | |
| | wild- | wild- | | | idtD, idtO, |
| | type | type | idtD | idtD, idtO | idtA |
| Paspaline ^{#} | 0.17 | 1.66 | 1.47 | 1.35 | 1.38 |
| Terpendole E ^{#} | 0.06 | 0.24 | 0.26 | 0.18 | 0.19 |
| Paspaline B ^{#} | 0.02 | 0.04 | 0.05 | 0.06 | 0.06 |
| Terpendole I ^{#} | 0.03 | 1.26 | 1.27 | 0.86 | 0.81 |
| Terpendole J ^{#} | 0.02 | 0.16 | 0.19 | 0.14 | 0.11 |
| Terpendole C ^{#} | 0.00 | 0.07 | 0.04 | 0.04 | 0.04 |
| Terpendole K ^{#} | 0.02 | 1.18 | 0.45 | 0.46 | 0.41 |
| Terpendole N ^{#} | 0.01 | 1.89 | 0.97 | 0.99 | 1.14 |
| Epoxy-Janthitriol ^{‡} | 4.8 | 0 | 0 | 9.5 | 4.4 |
| Epoxy-janthitrem I ^{‡} | 35.8 | 0 | 0 | 0.2 | 4.6 |
| Epoxy-janthitrem II ^{‡} | 12.9 | 0 | 0 | 5.0 | 3.6 |
| Epoxy-janthitrem III ^{‡} | 20.4 | 0 | 0 | 4.3 | 1.8 |
| Epoxy-janthitrem IV ^{‡} | 14.6 | 0 | 0 | 0 | 0.6 |

^{#} Values are paxilline standard equivalent
^{‡} Values are ug/g DM

As can be seen from Table 3 and Table 4, the expression of idtD and idtO together led to the production of epoxy-janthitriol, epoxy-janthitrem II, epoxy-janthitrem III, and trace levels of epoxy-janthitrem I. Without wishing to be bound by any theory, the inventors believe this may be due to the activity of a plant acylation enzyme acting on epoxy-janthitriol.

Expression of idtD, idtO and idtA together lead to the production of epoxy-janthitriol, epoxy-janthitrem I, epoxy-janthitrem II, epoxy-janthitrem III, and epoxy-janthitrem IV.

These data clearly establish the provision of epoxy-janthitrem production in a cell and organism that was not previously capable of producing such compounds, and thus the feasibility of conferring on an organism the ability to produce one or more epoxy-janthitrem compounds and so one or more attendant benefits associated with these compounds.

### Example 2

This example describes the analysis of the sequences of the idtA, idtD, and idtO genes from several representative strains of Epichloë festucae var. lolii, and of the polypeptides encoded thereby.

Methods

Genomic sequences were obtained from three isolates of Epichloë festucae var. lolii AR37 (AR37, AR37PP, and AR37S), Epichloë festucae var. lolii AR40, Epichloë festucae var. lolii AR127, Epichloë festucae var. lolii AR128, and Epichloë festucae var. lolii AR166.

Sequence analysis, including derivation of the amino acid sequences encoded by each of these genes, was performed using Geneious Prime bioinformatics software.

Results

The genomic sequences across the relevant section of the idt gene cluster was highly conserved among the AR37 (AR37, AR37PP, and AR37S), AR40, AR127, AR128, and AR166 strains.

Notably, 100% sequence identity was observed across all these strains for the idtA gene, including 1 kb upstream comprising the promoter through to the terminator.

A single nucleotide polymorphism (SNP) comprising a C->T substitution was identified in the promoter of the idtD gene in strains AR166 and AR128. Another SNP, comprising a C->A substitution, was identified in exonl of the idtD genes for strains AR127, AR128 and AR166. This results in an amino acid change from Aspartic Acid (D) to a Glutamic Acid (E) at amino acid position 235.

The analysis of the idtO genes from the various strains above identified the following variations:
deletion of a G in the promoter of the idtO gene of strains AR37 and AR40, compared to the remainder of these strains;
a SNP comprising an A->T substitution in the promoter for strains AR127, AR128 and AR166;
a SNP comprising a T->C substitution in the promoter for strains AR127, AR128 and AR166;
deletion of a C in intron 3 for strains AR37 and AR166, compared to the remainder of these strains;
a SNP comprising a T->G substitution in intron 3 for strain AR166.

The amino acid sequences encoded by the idtA, idtD, and idtO genes are highly conserved across these strains, with the aspartic acid to glutamic acid substitution in IdtD for strains AR127, AR128, and AR166 comprising a polypeptide variant.

### Example 3

This example describes the analysis of the sequences of other genes in the epoxy-janthitrem pathway, including idtF and idtK from several representative strains of Epichloë festucae var. lolii, and of the polypeptides encoded thereby.

Methods

Genomic sequences were obtained from Epichloë festucae var. lolii AR1, Epichloë festucae var. lolii AR5, Epichloë festucae var. lolii AR6, Epichloë festucae var. lolii AR48, Epichloë festucae var. lolii AR3060, Epichloë festucae var. lolii E2368, Epichloë festucae var. lolii Fg1, and Epichloë festucae var. lolii F11, and compared to one another and to Epichloë festucae var. lolii AR37.

Sequence analysis, including derivation of the amino acid sequences encoded by each of these genes, was performed using Geneious Prime bioinformatics software.

Results

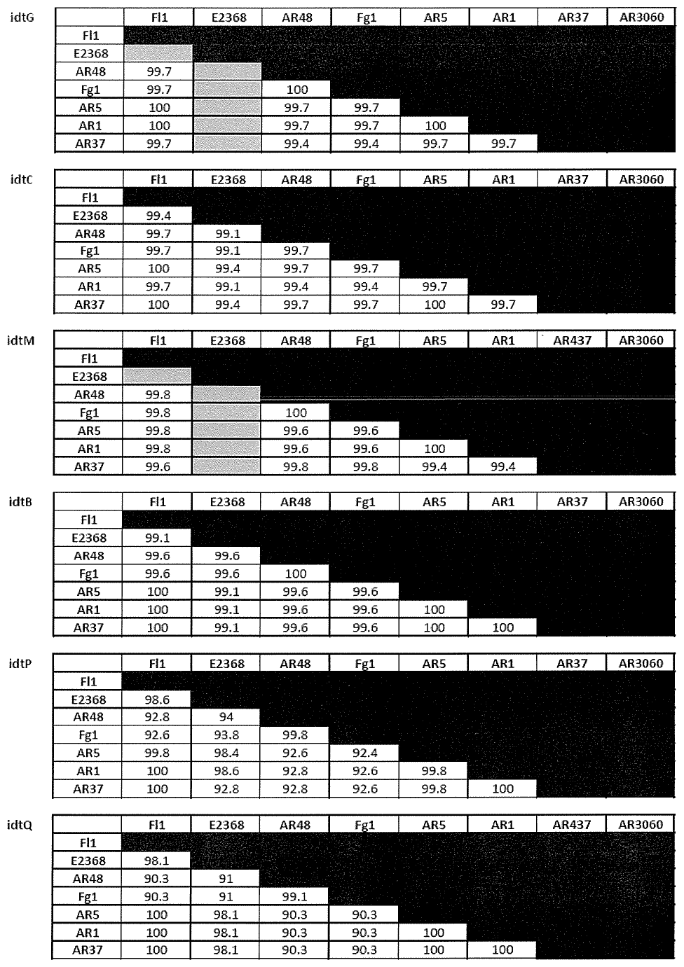

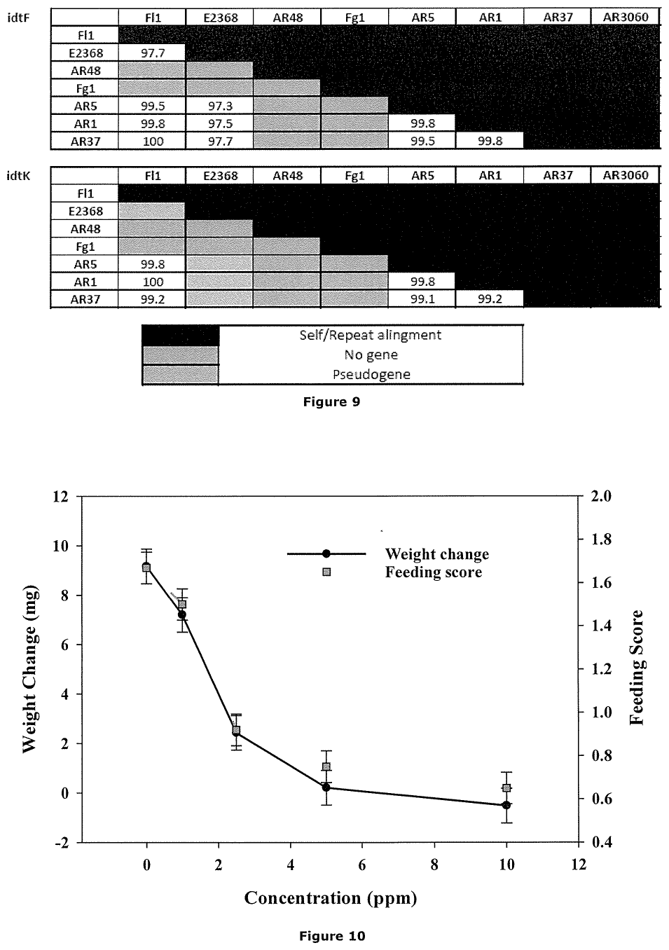

The derived amino acid sequences encoded by the genes idtG, idtM, idtB, idtC, idtP, idtQ, idtF, and idtK in the idt gene cluster for each strain was compared. As can be seen in FIG. 9, a high degree of amino acid sequence identity was observed for the genes idtG, idtM, idtB, idtC, idtP, idtQ, idtF, and idtK when these genes were present in a given strain. Notably, a number of pseudogenes were observed in these strains, together with a complete absence of one or more of these genes in several strains.

These strains are thus representative of host cells suitable for use as contemplated herein, whereby in addition to the modification or transformation to comprise at least one polynucleotide encoding the gene idtA, idtD, and/or idtO, the host cell is modified or transformed to comprise one or more polynucleotides selected from the group comprising: a polynucleotide encoding the gene idtG, a polynucleotide encoding the gene idtM, a polynucleotide encoding the gene idtB, a polynucleotide encoding the gene idtC, a polynucleotide encoding the gene idtP, a polynucleotide encoding the gene idtQ, a polynucleotide encoding the gene idtF, and a polynucleotide encoding the gene idtK, for example to provide the functionality of one or more polypeptides encoded by one of these genes and that is lacking in the strain, for example due to the absence of the gene or the presence of a pseudogene.

### Example 4

This example describes the assessment of insecticidal activity of purified epoxy-janthitrem I in a bioassay.

Methods

Isolation of Epoxyjanthitrem I for Bioassay

Epoxy-janthitrems were extracted from ground AR37-infected perennial ryegrass seed (cv. Extreme sourced from PGG Wrightson Seeds Limited, Christchurch, New Zealand) (70 g) with petroleum ether (40-60° C., 450 mL) by soxhlet extraction for 3 hours. After the extraction period, the seed was replaced with fresh seed (70 g) which was extracted for a further 3 hours. This process was repeated twice more to yield an extract resulting from 280 g of seed. The petroleum ether was then extracted with acetonitrile (2×200 mL) which was dried under reduced pressure to yield an epoxy-janthitrems fraction. Epoxy-janthitrems were found to be highly unstable so to minimize degradation, the fractions were kept on ice and were protected from light during all steps of the purification process. 2-Mercaptoethanol was also added to eluents at some stages of the purification to act as an anti-oxidant. To separate epoxy-janthitrem I from epoxy-janthitrems II-IV, flash column chromatography using silica gel (Merck, Art. 9385) was utilized. The sample was applied to a silica column (4×15 cm) in toluene which was then eluted with a gradient of toluene-acetone (100% toluene, 100 mL; 90% toluene, 200 mL; 85% toluene, 200 mL; 80% toluene, 200 mL; 75% toluene, 200 mL; 70% toluene, 200 mL; 50% toluene, 200 mL; 0% toluene, 200 mL). Fractions (13 mL) were collected and analyzed for epoxy-janthitrems which showed that epoxy-janthitrems II-IV eluted in the 90% toluene eluent whilst epoxy-janthitrem I eluted in the 85, 80 and 75% toluene eluent.

To further purify the fraction containing epoxy-janthitrem I, two additional silica flash columns were run (2.5×12 cm), one with an eluent of 17:3 toluene-acetone with the addition of 0.25% 2-mercaptoethanol and one with an eluent of 23:2 toluene-acetone, again with the addition of 0.25% mercaptoethanol. For each column, fractions were collected and analyzed by HPLC. The fractions found to contain epoxy-janthitrem I were then combined and dried down under reduced pressure. To further purify epoxy-janthitrem I, a C18 sep-pak was used (Waters Corporation, Mass., USA). The sep-pak was firstly flushed with acetonitrile (4 mL) and the sample applied in acetonitrile (0.5 mL). Acetonitrile (2 mL) was eluted and collected as the epoxy-janthitrems fraction followed by acetonitrile (1 mL) and acetone (3 mL) into waste. Aliquots (0.5 mL) of the total sample (2.5 mL) were processed in this way and the 2 mL acetonitrile fractions combined to yield the fraction containing epoxy-janthitrem I. This process was repeated following the same protocol to remove further contaminants. The final purification of epoxy-janthitrem I was achieved using two preparative HPLC procedures performed using a Waters series 600 controller and a Prodigy 5 μL ODS (3) HPLC column (250×10 mm) (Phenomenex, Torrance, Calif., USA). Epoxy-janthitrems were detected at 265 nm using a Waters 486 UV detector. The first procedure was conducted using an eluent of 19:1 acetonitrile-water at 5 mL/min. The peak representing epoxy-janthitrem I was detected by UV spectroscopy and was collected in a flask containing 2-mercaptoethanol. Aliquots of the sample were applied until the entire sample had been processed in this way. The resulting fraction containing epoxy-janthitrem I was dried down before conducting the second preparative HPLC procedure. In this case the same chromatographic equipment was used but this time the eluent utilized was 100% methanol at 4 mL/min and 2-mercaptoethanol was not added to the collection flask. The resulting epoxy-janthitrem I was analysed by NMR spectroscopy which showed it to be of >95% purity. An accurate weight was then obtained and aliquots prepared for testing in bioassays.

Porina Bioassay

Six week-old porina (W. cervinata) larvae reared from eggs (Popay, 2001) were starved overnight and then weighed individually. Larvae were assigned to replicates so that all larvae within a replicate were of a similar weight but randomised amongst treatments. The 20 replicate larvae per treatment were each placed in specimen containers (75 mL) half filled with commercially available fine bark chips.

An agar-based semi-synthetic diet (Popay, 2001) without antimicrobials was prepared and epoxy-janthitrem I dissolved in 100 μL DMSO was added to 10 g of diet. Four concentrations of epoxy-janthitrem I were prepared (1, 2.5, 5, 10 μg/g wet weight) along with a DMSO control. Discs of diet (7 mm diam.) were taken and placed on top of the bark and the lids were replaced. The experiment was kept in the dark at 15° C. After 24 h, each diet was visually scored for feeding on a scale of 0-10 where 0=no feeding and 10=all diet consumed. The amount of webbing present was also scored on a scale of 0-5. The diet disc was replaced daily with fresh diet for 7 days. Each time diet was replaced, samples of the diet that the porina had fed on were taken as well as a sample of the fresh diet to analyse for epoxy-janthitrem I. Porina were reweighed at the completion of the trial.

The feeding score data were normalised by square root transformation before a repeated measures analysis was carried out using REML with treatment and date as factors. The daily webbing score and weight change of larvae over 7 days were analysed using ANOVA, blocked by replicate, to make pairwise comparisons for each concentration with the solvent control at each date.

Results

Analysis of porina diets showed that for the experiment incorporating pure epoxy-janthitrem I into insect diets, the concentrations were as expected when analysed at time zero (average of 98%) showing that epoxy-janthitrem I was stable during the diet-making process. However, analysis of diets after 24 h showed some decomposition of epoxy-janthitrem I over this time period (average of 81% of the starting concentration).

Average weight of larvae at the beginning of the experiment was 14.7 mg with a range of ±0.5 mg across treatments. Survival of larvae during the experiment was 95% in the solvent control and 100% in treatments containing epoxy-janthitrem I. Apart from the first day, the feeding score of porina fed epoxy-janthitrem I at 1 ppm did not differ significantly from the solvent control during the experiment and nor did the average feeding score (FIG. 10). For porina fed epoxy-janthitrem I at 2.5 ppm there was a consistent reduction in feeding score for the first 6 days and for porina fed epoxy-janthitrem I at 5 and 10 ppm feeding was strongly inhibited for the entire period of the bioassay. Weight change continued to decline with increasing concentration (FIG. 10). Webbing scores followed a very similar pattern (data not presented) indicating that this was strongly linked to the deterrent effect of the diet and reduced consumption.

Weight gain of larvae over the course of the experiment was significantly reduced relative to the solvent control at all concentrations except 1 ppm which reflected the feeding scores. However, calculating weight gain as a percentage of that in the solvent control showed a significant effect at 1ppm (% gain in weight for initial solvent control 63.4% and 1 ppm epoxy-janthitrem I 51.1%; P<0.05), suggesting some activity even at this low concentration.

Based on the dry weight of diet, the concentrations tested in this experiment are approximately ten times the wet weight concentrations, indicating that an in planta concentration of 25 ppm epoxy-janthitrem I and above will reliably reduce larval feeding but that lower concentrations may have only a minor effect.

These data clearly establish that purified epoxy-janthitrem I is effective in the biological control of Porina larvae, supporting the use of epoxy-janthitrems in the control of important pasture pests. Further, these data show that at least some of the in planta toxicity observed against Porina larvae is attributable to epoxy-janthitrem I.

### Example 5

This example describes the assessment of mammalian bioactivity of purified epoxy-janthitrem I in a bioassay.

Methods

The epoxy-janthitrems used in this mammalian bioassay were isolated as described above in Example 4.

Mouse Bioassay

Epoxy-janthitrem I, lolitrem B, and the related indole-diterpene, paxilline were administered by intraperitoneal injection as DMSO/water solutions (9:1, 50 μL) into mice (Swiss, female, weight 25+3 g). Control mice were injected with the solvent alone. Dose rates which induce an acceptable maximum tremor response are well established for lolitrem B (2 mg/kg; Gallagher and Hawkes, 1986) and paxilline (6 mg/kg; Miles, et al., 1992).

A starting dose rate of 8 mg/kg was used for epoxy-janthitrem I which gave only a low tremor response, a dose rate of 14 mg/kg was therefore subsequently used for the experiment. Groups of four mice were used for each treatment group. All animal manipulations were approved by the

AgResearch Ruakura (Hamilton) Animal Ethics Committee established under the Animal Protection (code of ethical conduct) Regulations Act, 1987 (New Zealand). Tremor score was rated 1-5 on the basis of a visual rating scale using well established protocols (Gallagher and Hawkes, 1985; Gallagher and Hawkes, 1986). Tremors were measured regularly until the mice had completely recovered.

Results

Initially, epoxy-janthitrem I was dosed at 8 mg/kg but this was found to give only a low tremor response. Subsequently, groups of four mice were dosed with epoxy-janthitrem I at 14 mg/kg, lolitrem B at 2 mg/kg, paxilline at 6 mg/kg, or with solvent alone.

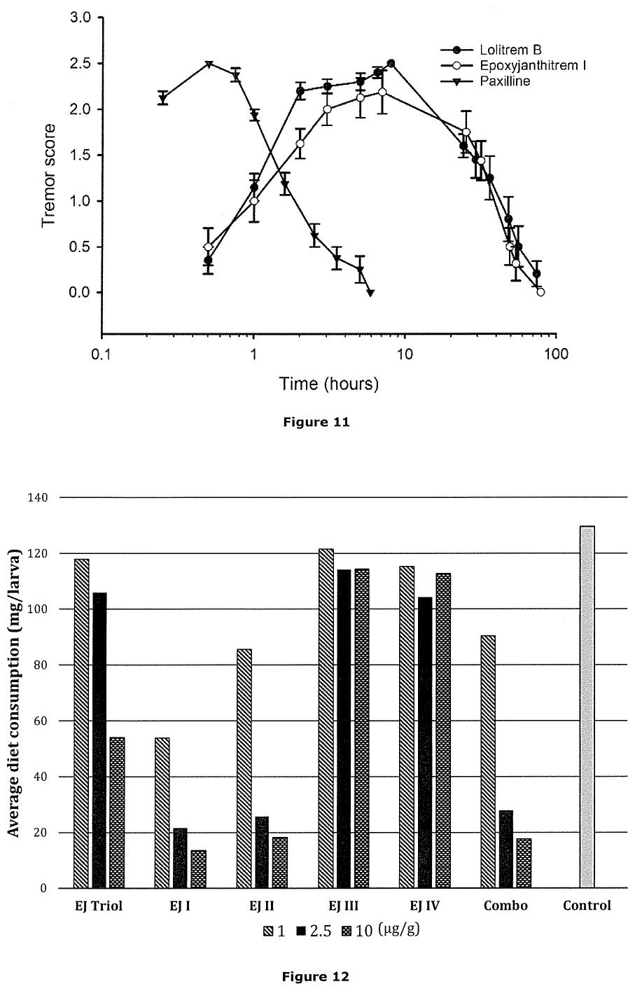

No tremors were observed in control mice, but a tremor response was observed in all of the other treatment groups (FIG. 11). The time course of action for epoxy-janthitrem I was found to be unlike that of paxilline or for those reported for janthitrems and the epoxyjanthitrem isolated from P. janthinellum (Babu, et al., 2018). In these cases, tremors peaked at 30 mins post-dosing and the mouse had completely recovered by 6 hours post-dosing. In contrast, mice dosed with either epoxy-janthitrem I or lolitrem B showed tremors peaking at 7-8 hours post-dosing which persisted for over 2 days.

The sustained tremor effect induced in mice dosed with epoxy-janthitrem I is comparable to that observed in mice dosed with lolitrem B, albeit only when epoxy-janthitrem I is dosed at a significantly higher dose rate.

### Example 6

This example describes the assessment of insecticidal activity of purified epoxy-janthitrems in a model insect bioassay.

Methods

Epoxy-janthitrem I used in this bioassay was isolated as described above in Example 4. The purification process was conducted on a total of 280 g of seed to yield a bulk fraction containing epoxy-janthitrem I and epoxy-janthitriol, and a bulk fraction containing epoxy-janthitrems II, III, and IV, which were each then further purified by preparative HPLC. This was performed using a Waters series 600 controller and a Prodigy 5 μm ODS (3) HPLC column (250×10 mm) (Phenomenex, Torrance, Calif., USA) fitted with a 10×10 mm Phenomenex Security Guard™ (Torrance, Calif., USA). Epoxyjanthitrems were detected at 265 nm using a Waters 486 UV detector. Initial removal of contaminants from the mixture of epoxy-janthitrem I and epoxy-janthitriol was achieved using an eluent of 19:1 acetonitrile-water, 10 mL/min. The two epoxyjanthitrem compounds were then separated using an eluent of 9:1 acetonitrile-water, 10 mL/min.

Epoxy-janthitrems II, III, and IV were separated using an eluent of 100% acetonitrile, 10 mL/min. Using this solvent system, epoxy-janthitrem II and III were not completely resolved, which necessitated further purification of epoxy-janthitrem II using an eluent of 19:1 acetonitrile-water, 10 mL/min. The preparative HPLC purifications detailed above resulted in five fractions each containing an individual epoxyjanthitrem compound. Final purification was performed on each individual fraction by performing two preparative HPLC steps using 100% methanol, 10 mL/min as the eluent. NMR analysis showed each epoxyjanthitrem compound to be of high purity and suitable for complete structural elucidation and to allow a full NMR assignment to be made.

The porina bioassay was carried out as described in Example 4, with the following variations:

15 replicates were used per treatment;

the age of the larvae at the start of the experiment was 10 weeks;

diet was made weekly and replaced after 4 days within each week;

the average weight of diet consumed by the larvae, larval weight gain/loss (data not shown), and larval mortality (data not shown) was determined;

three concentrations of purified epoxy-janthitrems were used (1, 2.5, 10 μg/g wet weight) instead of four; and

an average daily diet consumption (mg/larva) across the 21 days of the trial was determined.

Results

As can readily be seen in FIG. 12, average diet consumption decreased with increasing epoxy-janthitrem concentration for epoxy-janthitrem I, epoxy-janthitrem II, epoxy-janthitriol, and the combination of epoxy-janthitrems. Even at the lowest dose, epoxy-janthitrem I and to a lesser degree epoxy-janthitrem II, had a substantial inhibitory effect on consumption. In contrast, epoxy-janthitrem III and epoxy-janthitrem IV showed little inhibition of feeding by Porina larvae, even at the highest concentration. Weight change of larvae followed the feeding pattern, with a net weigt loss at 2.5 μg/g for epoxy-janthitrem I, epoxyjanthitrem II and a combination of all epoxy-janthitrems.

These data clearly establish that purified epoxy-janthitrem I and epoxy-janthitrem II are both highly effective at deterring feeding by Porina larvae, supporting the use of these epoxy-janthitrems in the control of important pasture pests. Mortality of larvae was high for larvae fed 2.5 and 10 μg/g of epoxy-janthitrem I, epoxyjanthitrem II and a combimation of all epoxy-janthitrems (data not shown).

### Example 7

This example describes the assessment of mammalian bioactivity of purified epoxy-janthitrems in a bioassay.

Methods

The epoxy-janthitrems used in this mammalian bioassay were isolated as described above in Example 4.

Mouse Bioassay

Epoxy-janthitrem I, Epoxy-janthitrem II, Epoxy-janthitrem III, Epoxy-janthitrem IV, and Epoxy-janthitriol, were administered by intraperitoneal injection as DMSO/water solutions (9:1, 50 μL) into mice (Swiss, female, weight 25+3g). Control mice were injected with the solvent alone (data not shown). Groups of four mice were used for each treatment group. All animal manipulations were approved, and tremor score was calculated, as described above in Example 5.

The dosage rates were informed by the experiment described in Example 5 above, with dose rates as follows: epoxy-janthitrem I (EJ I, 13.5 mg/kg), epoxy-janthitrem II (EJ II, 14.2 mg/kg), epoxy-janthitrem III (EJ III, 17.2 mg/kg), epoxy-janthitrem IV (EJ IV, 16.5 mg/kg), epoxy-janthitriol (EJ triol, 16.2 mg/kg). Tremors were measured regularly until the mice had completely recovered.

Results

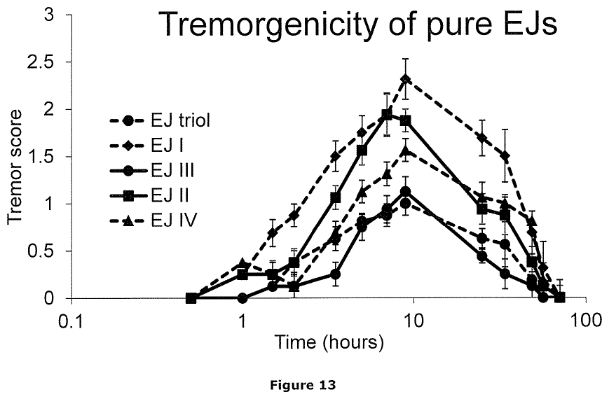

As can readily be seen in FIG. 13, a tremor response was observed in all treatment groups. The time course of action for all epoxy-janthitrems other that epoxy-janthitrem II was comparable, with tremorgenicity peaking at 10 hours post administration. A slightly earlier peak in tremorgenicity, at 9 hours, was observed for epoxy-janthitrem II, but applicants believe, without wishing to be bound by any theory, that this difference in peak tremorgenicity is unlikely to be significant. Notably, for each treatment group, tremor persisted for over 2 days.

Of interest is the substantial difference in tremor score observed with the different epoxy-janthitrem compounds. While the epoxy-janthitrems were dosed at slightly different rates, the applicants believe, again without wishing to be bound by any theory, that any dosage difference is not sufficient to account for the observed differences in effect. For example, despite being dosed at the lowest rate, the peak tremor score observed with epoxy-janthitrem I was significantly greater than that observed for any of the other epoxy-janthitrems. The lowest peak tremor scores were observed for epoxy-janthitrem III and epoxy-janthitriol.

These data clearly establish that the different individual epoxy-janthitrems have differing tremorgenic activity in a mammalian model, thus showing that the different epoxy-janthitrems exhibit differing mammalian toxicity.

### Example 8

This example describes the generation of heterologous hosts capable of producing for the first time one or more epoxy-janthitrem compounds, and the characterisation of epoxy-janthitrem compound production in these hosts.

Methods

Bacterial Strains

Escherichia coli strain Top10 (Invitrogen Corp., Carlsbad, Calif., USA) was grown on Luria-Bertani broths and agar plates supplemented with ampicillin (100 μg/mL).

Fungal Strains and Growth Conditions

Cultures of the Epichloë taxonomic group LpTG-3 strain AR37, Epichloë festucae var. lolii strain AR1, E. coenophiala strain AR584 and E. bromicola strains AR3028 and AR3056, were maintained on 1.5% (w/v) potato dextrose agar (PDA) (Difco, Sparks, Md., USA) supplemented where necessary with hygromycin (150 μg/mL).

Endophyte Inoculation

Endophyte-free seedlings were inoculated using the method of Latch and Christensen (Latch and Christensen, 1985) as follows: Perennial ryegrass (Lolium perenne cv. Samson) was inoculated with Epichloë festucae var. lolii strain AR1 wild type and the same strain transformed with the idtO, idtD and idtA genes from strain AR37. Endophyte-free seedlings of Tall fescue (Festuca arundinaceae cv. Hummer) were inoculated with E. coenophiala strain AR584 wild type and the same strain transformed with the idtO, idtD and idtA genes from strain AR37. Endophyte-free seedlings of Rye (Secale cereale cv. Rahu) were inoculated with E. bromicola strains AR3028 wild type and 3056 wild type and the same strains transformed with the idtO, idtD and idtA genes from strain AR37. Seedlings were grown in proprietary potting mixture in 90 cm pots under glasshouse conditions for 6 weeks and assessed for endophyte infection by immunoblotting (Simpson et al., 2012).

Genomic DNA and Plasmid Isolation

Genomic DNA was isolated from freeze-dried Epichloë mycelium as described in Byrd et al., 1990. Plasmid DNA was isolated and purified using a plasmid mini kit (ZymoPure).

Preparation of the Gene Insertion Constructs

Linear PCR products used for transformation were amplified using PrimeSTAR polymerase (Takara) from AR37 genomic DNA using the PCR primers idtA 95889F, idtA 98203R, idtD MG159, idtD MG158, idtO 48763F, idtO 51896R (as depicted in Example 1, Table 1 above) for the genes idtA, idtD and idtO, respectively.

Fungal Protoplasting and Transformation

Protoplasts of Epichloë festucae var. lolii strain AR1, E. coenophiala strain AR584 and E. bromicola strains AR3056 and AR3028 were prepared as described in Fleetwood et al., 2007. Fungal cultures were grown in 50 mL defined media inoculated with macerated fungi (grown on cellophane PDA plates for 7 days at 22 ° C.) and grown at 22 ° C. with moderate shaking (150 rpm) for 5 days. The mycelial pellets were washed with 1 L sterile H₂O followed by a wash in OM buffer (1.2 M MgSO₄, 10 mM Na₂HPO₄, pH 5.8). The washed mycelia were mixed with 30 mL of sterile OM buffer containing 15 mg/mL trichoderma lysing enzyme (Sigma, St Louis, Mo., USA). This mixture was shaken gently (100 rpm) for 18 h at 30 ° C. The digested hyphae were filtered through Mira-cloth (Calbiochem, San Diego, Calif., USA) and the filtrate, containing the protoplasts, was overlaid with 2 mL STC buffer (0.6 M sorbitol, 100 mM Tris-HCL pH 8.0). The protoplasts were banded at the interface by centrifugation at 3000×g for 15 min, washed three times with 10 mL STC buffer (1 M sorbitol, 50 mM CaCl₂, 50 mM Tris/HCl pH8.0) by centrifuging at 7700x g and resuspended in STC buffer to a final concentration of 1.25×10⁸ per mL. Protoplasts were transformed as described in Fleetwood et al., 2007 with 300 fmol linear DNA PCR product—co-transformed with 750 ng of plasmid pN1688 containing the hygromycin selectable maker. Transformants were selected on RG Media (PD with 0.8 M sucrose pH 6.5) containing hygromycin (150 μg/mL). To obtain clonal isolates, the resulting transformants were purified by sub-culturing three times as described by Young et al., 2005.

Molecular Analysis of Transformants

The idtO, idtD and idtA gene insertion transformants were screened by PCR for their random integration into AR1, AR584, AR3028 and AR3056 with the primers used to make the insertion PCR products.

Results:

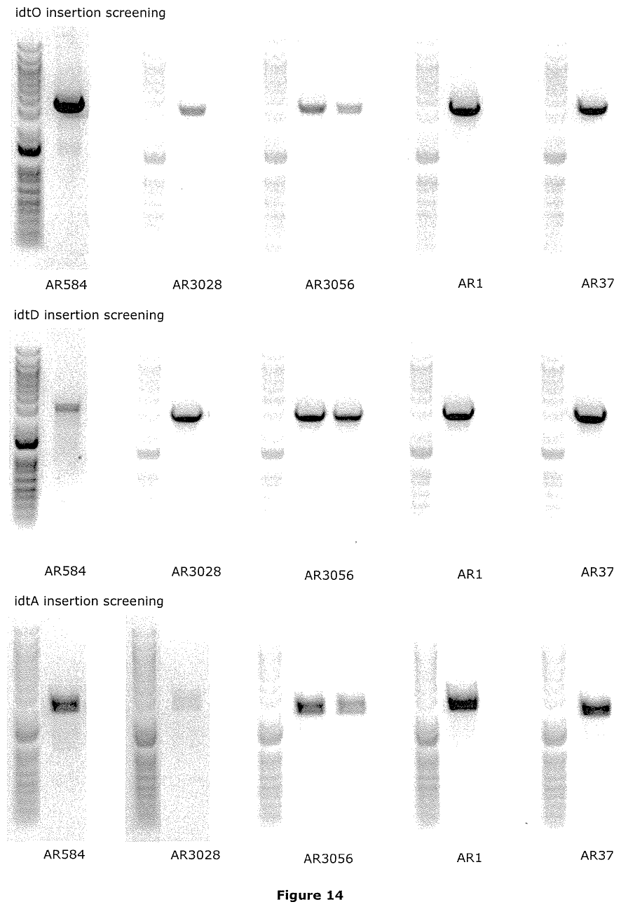

FIG. 14 clearly demonstrates that stably inserted idtO, idtD, and idtA genes were present in the various target Epichloë strains. The production of PCR products with the appropriate specific primers was clearly visible in each strain into which the idtO gene (FIG. 14, top), the idtD gene (FIG. 14, middle), or the idtA gene (FIG. 14, bottom), had been inserted.

Table 5 below presents the results of LC/MS assays (as described in Example 1) to determine the presence of epoxy-janthitrem in samples of harvested symbionts, along with controls. Wild type AR37 (AR37 WT), an Epichloë strain capable of natively producing epoxy-janthitrem compounds as described herein, was used as positive control (AR37 WT). Epichloë-free cultivar (E free) was used as a negative control.

As can clearly be seen in Table 5, the ability to produce one or more epoxy-janthitrem compounds was successfully conferred on certain of these target Epichloë strains. Strains AR6 and AR584 were robust producers of all epoxy-janthitrem compounds. Furthermore, the data in Table 5 establishes that Epichloë strains capable of producing selected epoxy-janthitrem compounds, and not others, have been prepared. Two different AR3056 transformants were each capable of producing epoxy-janthitriol, and AR3056 #2 additionally produced epoxy-janthitrem I, but neither transgenic AR3056 strain produced epoxy-janthitrem II, III, or IV, nor was peramine produced.

TABLE 5
Production of indole diterpene compounds in transgenic strains

| | | Indole diterpene compound (mg/kg dry matter) | | | | | |
|---|---|---|---|---|---|---|---|
| Strain | Cultivar | Etriol | EJI | EJII | EJIII | EJIV | Peramine |
| AR37 WT | GA66 | 5.3 | 78.6 | 102.7 | 25.2 | 22.4 | 0 |
| AR6 WT | GA66 | 0 | 0 | 0 | 0 | 0 | 40.8 |
| AR6 idtDOA | GA66 | 7.3 | 14.6 | 32.3 | 1.9 | 0.8 | 37.7 |
| E free | GA66 | 0 | 0 | 0 | 0 | 0 | 0 |
| AR584 WT | Hummer | 0 | 0 | 0 | 0 | 0 | 33.7 |
| AR584 | Hummer | 0.7 | 1.7 | 25.4 | 1.5 | 0.2 | 29.5 |
| idtDOA | | | | | | | |
| E free | Hummer | 0 | 0 | 0 | 0 | 0 | 0 |
| AR3056 WT | Rahu | 0 | 0 | 0 | 0 | 0 | 0 |
| AR3056 | Rahu | 5.4 | 0 | 0 | 0 | 0 | 0 |
| idtDOA #1 | | | | | | | |
| AR3056 #2 | Rahu | 1.3 | 0.3 | 0 | 0 | 0 | 0 |
| E free | Rahu | 0 | 0 | 0 | 0 | 0 | 0 |

These data clearly establish that the methods exemplified here enable the production in a transgenic host of one or more epoxy-janthitrem compounds, including the production of one or more desired epoxy-janthitrem compounds without the concomitant production of one or more other epoxy-janthitrem compounds or with reduced production of one or more other epoxy-janthitrem compounds, and/or the production of one or more desired epoxy-janthitrem compounds without the concomitant production of one or more less desirable compounds, or with reduced production of one or more less desirable compounds.

### Example 9

This example describes the generation of modified hosts in which epoxy-janthitrem production has been modified, and the characterisation of epoxy-janthitrem compound production in said hosts.

Materials and Methods

Bacterial strains, fungal strains, growth conditions therefor, isolation and cloning methods, and protoplasting and transformation were as described above in Example 8, with the following additional details.

Perennial ryegrass (Lolium perenne cv. Samson) was inoculated with Epichloë taxonomic group LpTG-3 strain AR37 wild type and independent epoxy-janthitrem idtD or idtA CRISPR-Cas9 gene edited mutants. Seedlings were again grown in proprietary potting mixture in 90 cm pots under glasshouse conditions for 6 weeks and assessed for endophyte infection by immunoblotting as set out in Example 8 above.

CRISPR-Cas9 Protospacer Design and Cloning

The AR37 epoxy-janthitrem idtA and idtD genes were screened for CRISPR-Cas9 target sites with the protospacer adjacent motif (PAM (NGG)) sequence using Geneious (Biomatters Ltd). Two protospacer sequences (with no predicted off-site targets) targetting each gene were selected. Table 6 below presents the sequences of these protospacer sequences, in which the PAM (NGG) sequence is shown in parentheses.

TABLE 6
CRISPR-Cas9 target sites (PAM (NGG)-AR37 idtA
& idtD

| | | SEQ ID |
|---|---|---|
| Primer | Sequence | NO: |
| idtD g63 | GGA AGA GCT TAG ATA CAA AG (TGG) | 24 |
| idtD g148 | CTT CCT GGA TCG AGA TAC GG (CGG) | 25 |
| idtA g64 | GCA TGG CCT CGG GAA GGT GA (GGG) | 26 |
| idtA g101 | GGA GCC AGC GAT ACA AAG GG (CGG) | 27 |

Construction of Plasmid ANEp8-Cas9-idtDg63 and ANEp8-Cas9-idtDg148

The ANEp8-Cas9-LIC1 plasmid was used as host vector to harbour the gRNA cassette by using the ligation independent cloning (LIC) method. The idtD gRNA cassette for each target site (idtD g63, idtD g148) used for plasmid construction was amplified with a pair of end primers to link with LIC sequence sites at both sides, to generate complementary single-strand overhangs between ANEp8-Cas9 vector and the idtD gRNA cassette insert. The sequence of these LIC primers is shown in Table 7 below.

TABLE 7
LIC oligonucleotides-idtD

| Primer | Sequence | SEQ ID NO: |
|---|---|---|
| idtD g63 Rev P1 | CTT TGT ATC TAA GCT CTT CCG ACG AGC TTA CTC GTT TCG | 28 |
| idtD g63 Fw P1 | GGA AGA GCT TAG ATA CAA AGG TTT TAG AGC TAG AAA TAG CAA G | 29 |
| idtD g148 Fw P1 | CTT CCT GGA TCG AGA TAC GGG TTT TAG AGC TAG AAA TAG CAA G | 30 |
| idtD g148 Rev P1 | CCG TAT CTC GAT CCA GGA AGG ACG AGC TTA CTC GTT TCG | 31 |

The Swal linearized ANEp8-Cas9 and the idtD gRNA cassette DNA (ending with LIC tails) were treated by T4 DNA polymerase in the presence of dGTP and dCTP, respectively. The 20 μL reaction mixture contained 0.2 pmol of DNA, 0.8 pL of 100 mM dithiothreitol, 2 μL of 25 mM dGTP or dCTP, and 3 U of T4 DNA polymerase in NEB buffer 2.1. The reaction was carried out at 22° C. for 30 minutes followed by enzyme inactivation by heating at 75° C. for 20 minutes. The insert and vector were mixed in a 3:1 molar ratio. To achieve annealing, the mixture was first heated at 60° C. for 5 minutes and then gradually decreased to 4° C. (reduce 0.1° C. per second). The annealed products were transformed into E.coli Top10 (Invitrogen) competent cells to generate plasmid ANEp8-Cas9-idtDg63 and ANEp8-Cas9-idtDg148.

Construction of Plasmid ANEp8-Cas9-idtAg64 and ANEp8-Cas9-idtAg101

Primers were designed for each protospacer of the idtA gene (see Table 8 below), with the appropriate SapI overhang for cloning into the pCas9HygAMA-ccdB vector (see vector construction below).

TABLE 8
Protospacer oligonucleotides-idtA

| | | SEQ ID |
|---|---|---|
| Primer | Sequence | NO: |
| idtA g101 Top | GTC GGA GCC AGC GAT ACA | 32 |
| | AAG GG | |
| idtA g101 Btm | AAC CCC TTT GTA TCG CTG | 33 |
| | GCT CC | |
| idtA g64 Top | GTC GCA TGG CCT CGG GAA | 34 |
| | GGT GA | |
| idtA g64 Btm | AAC TCA CCT TCC CGA GGC | 35 |
| | CAT GC | |

Each protospacer was generated by annealing 15 ng of the forward and reverse oligonucleotides in annealing buffer (10 mM Tris-HCl pH 8, 50 mM NaCl, 1 mM EDTA, pH 8). The following thermocycler program was used for annealing: 5 min at 95° C., 20 sec at 95° C., a decrease of 0.5° C./cycle for 140 cycles, 1 min at 25° C. The annealed oligonucleotides were ligated with the digested Cas9HygAMA-ccdB (SapI) plasmid using T4 ligase (Invitrogen) at 20° C. for 15 minutes. The annealed products were transformed into E.coli Top10 competent cells to generate ANEp8-Cas9-idtAg64 and ANEp8-Cas9-idtAg101.

Transformants were screened by colony PCR using Sapphire Amp Fast PCR master mix (Takara) with the idtA gRNA Top anneal oligonucleotide and the reverse gRNA-specific primer gRNA Screen R. Sequencing to confirm the correct gRNAs was achieved by primer PCR using the gRNA screen F and gRNA screen R primers. The sequence of these primers is shown below in Table 9.

TABLE 9
Screening primers

| | | SEQ ID |
|---|---|---|
| Primer | Sequence | NO: |
| gRNA Screen | GGG GAT CAT AAT AGT ACT | 36 |
| R | AGC CA. | |
| gRNA screen | TTT TCT CTT CCA TTT ACG | 37 |
| F | C | |

Fungal Protoplasting and Transformation

Protoplasts of Epichloë taxonomic group LpTG-3 strain AR37 were prepared and transformed as described above in Example 8, albeit with 300 fmol of the appropriate Cas9HygAMAgRNA vector instead of the linear DNA/plasmid pN1688 mix. Transformants were again selected on RG Media (PD with 0.8 M sucrose pH 6.5) containing hygromycin (150 μg/mL). To obtain clonal isolates, the resulting transformants were purified by sub-culturing three times as described by Young et al., 2005.

Molecular Analysis of Transformants

AR37 transformants were screened for CRISPR-cas9 idtD and idtA gene editing events by sequencing a PCR product comprising the targeted CRISPR gene edit site. The PCR product was amplified using PrimeSTAR GXL polymerase (Takara) using screening primers designed to anneal to sequences up to 1 kb flanking the target PAM site. The sequences of these screening primers are shown in Table 10 below.

TABLE 10
CRISPR edit screening primers

| | | SEQ ID |
|---|---|---|
| Primer | Sequence | NO: |
| idtD64315F | ACC CCA AGA TTG CAT CCC AG | 38 |
| idtD64747R | GGC GGT GAT CTG TTC TGG AA | 39 |
| idtA96602F | GGG ACA GAC GTT AGC ACT CC | 40 |
| idtA97259R | CCC TAC CGT GCT GAA GAG TG | 41 |

Adaptions to the ANEp8_Cas9_LIC1 Plasmid (Concordia):

The ANEp8_Cas9_LIC1 plasmid (obtained from Concordia University; Aslandis & Jong, 1990; Storms, et al., 2005; Song, et al., 2018) was adapted to contain a hygromycin cassette in place of the pyrG gene for selection. The 15.6 Kb ANEp8_Cas9_LIC1 plasmid was initially digested with NotI to liberate a 5.3 kb fragment containing the AMA1 cassette (purified by gel extraction) and a 10.3 Kb fragment (purified by gel extraction) containing the Cas9 and pyrG genes. Subsequent digestion of the 10.3 kb fragment with Kpnl liberated a 9 Kb Cas9 cassette (purified by gel extraction) containing the Cas9 gene and removal of pyrG. To amplify the hygromycin resistance cassette (2.9 Kb truncated version), primer PCR was performed on pDONR₂₂₁-Hyg template with restriction enzyme adapted primers, the sequence of which is shown in Table 11 below.

TABLE 11
Hyg primers

| Primer | Sequence | SEQ ID NO: |
|---|---|---|
| KpnI TtrpC Hyg DONR R | GCC GGT ACC GCG CTT ACA CAG TAC ACG AG | 42 |
| NotI PgpdA Hyg pDONR F | AAG GAA AAA AGC GGC CGC CTA AAA TCC GCC GCC TCC AC | 43 |

The resulting product was digested with KpnI and NotI prior to ligation. The Cas9 (NotI/KpnI digested) cassette and the Hygromycin resistance (NotI/KpnI digested) cassette were ligated with T4 ligase (Invitrogen) at 16° C. overnight, creating the Cas9Hyg plasmid. The Cas9Hyg plasmid was re-digested with Notl and alkaline phosphatase treated (purified by gel extraction) before its T4 ligation with the AMA1 (Notl digested) cassette, creating the Cas9HygAMA plasmid.

Modification to the gRNA protospacer was achieved through Ligation Independent Cloning (LIC; Song et al., 2018). A mock gRNA protospacer, containing two Sapl restriction enzyme sites, was cloned into the Cas9HygAMA plasmid creating the Cas9HygAMASapI plasmid and thus removing the need for the use of LIC with future protospacers. The sequences of the primers required for the Sapl insertion using the LIC method are shown in Table 12 below.

TABLE 12
SapI insertion primers

| Primer | Sequence | SEQ ID NO: |
|---|---|---|
| SapI site CRISPR | GTC GGA AGA GCA AAA TGC TCT TCA GTT TTA GAG CTA GAA ATA | 44 |
| Fw P1 | GCA AG | |
| SapI site CRISPR | AAC TGA AGA GCA TTT TGC TCT TCC GAC GAG CTT ACT CGT TTC G | 45 |
| Rev P1 | | |
| Fw LIC2 | CAA CCT CCA ATC CAA TTT GAC TCC GCC GAA CGT ACT GG | 46 |
| Rev LIC2 | ACT ACT CTA CCA CTA TTT GAA AAG CAA AAA AGG AAG GTA CAA | 47 |
| | AAA AGC | |

A ccdB lethal cassette was cloned between the two Sapl sites to aid in the efficiency of the future protospacer cloning. To amplify the ccdB lethal cassette sequence (2 Kb), primer PCR was performed on the split marker vector pDONR-SM1 (Rahnama, et al., 2017) template with SapI restriction enzyme adapted primers, the sequences of which are shown below in Table 13.

TABLE 13
SapI restriction adapted primers

| Primer | Sequence | SEQ ID NO: |
|---|---|---|
| SapI ccdB F | TCG GCT CGT CGG AAG AGC GAC CGA CAG CCT TCC AAA TG | 48 |
| SapI ccdB R | TCG CTA AAA CTG AAG AGC GTT GGC AGC ATC ACC CGA CG | 49 |

The resulting product was digested with Sapl prior to its ligation with Cas9HygAMASapI (SapI digested), creating the Cas9HygAMAccdB plasmid.

Results:

CRISPR Gene Rdits Sequence Modifications

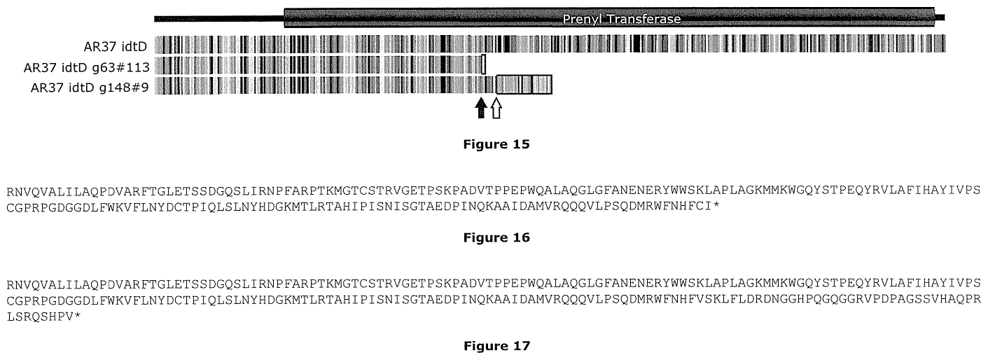

Depictions of the predicted truncated IdtD polypeptides resulting from the AR37 idtD g63 and g148 edits are presented in FIG. 15. The sites of the g63 and g148 edits are shown with a solid arrow, and an outlined arrow, respectively. Substituted amino acids resulting from the edits are boxed. The relative sizes of the truncated protein products resulting from each premature STOP codon are clearly visible.

The AR37 idtD g63 edit (the insertion of a T between C591 and T592 of SEQ ID NO: 5) leads to the substitution of two different amino acids followed by a premature STOP codon, as depicted in FIG. 16 and presented herein as SEQ ID NO: 50.

The AR37 idtD g148 edit (the insertion of an A between A622 and C623 of SEQ ID NO: 5) leads to the substitution of 33 different amino acids followed by a premature STOP codon, as depicted in FIG. 17 and presented herein as SEQ ID NO: 51.

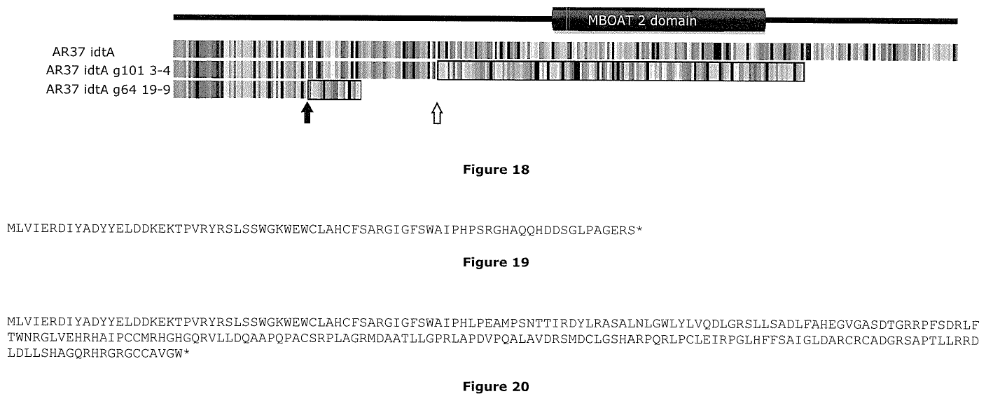

Depictions of the predicted truncated IdtA polypeptides resulting from the AR37 idtA g64 and g101 edits are presented in FIG. 18. The sites of the g64 and g101 edits are shown with a solid arrow, and an outlined arrow, respectively. Substituted amino acids resulting from the edits are boxed. The relative sizes of the truncated protein products resulting from each premature STOP codon are clearly visible.

The AR37 idtA g64 edit (the insertion of a T between A164 and C165 of SEQ ID NO: 2) leads to the substitution of 20 different amino acids followed by a premature STOP codon, as depicted in FIG. 19 and presented herein as SEQ ID NO: 52.

The AR37 idtA g101 edit (the deletion of AA between A318 and G321 of SEQ ID NO: 2) leads to the substitution of 147 different amino acids followed by a premature STOP codon, as depicted in FIG. 20 and presented herein as SEQ ID NO: 53.

Results:

Table 14 below presents the results of LC/MS assays (as described in Example 1) to determine the presence of epoxy-janthitrem compounds in samples of symbionts infected with the CRISPR-edited transgenic AR37 hosts (AR37 idtD CRISPR edit, AR37 idtA CRISPR edit), and in symbionts infected with wild type AR37.

As can clearly be seen from the data in Table 14 each CRISPR-mediated edit to the endogenous idtD gene in AR37 ablated epoxy-janthitrem production. Furthermore, an increase in terpendole precursors was observed.

CRISPR-mediated edits to the endogenous idtA gene in AR37 substantially reduced the production of certain epoxy-janthitrem compounds, notably epoxy-janthitrem I and epoxy-janthitrem IV. Production of epoxy-janthitrem III was not markedly affected, but that of epoxy-janthitrem II was decreased compared to the wild-type AR37. Production of epoxy-janthitriol was increased compared to that observed with wild-type AR37. Inactivation of idtA had substantially less effect on the levels of terpendole precursors when comprared to wild-type AR37 than was observed in the AR37 idtD CRISPR edit strain.

TABLE 14
Indole diterpene production in CRISPR edited AR37

| | Concentration of indole diterpene compound (mg/kg) | | | | | | | | | | | |
|---|---|---|---|---|---|---|---|---|---|---|---|---|
| | | | | | | | | Expoxy- | Expoxy- | Expoxy- | Expoxy- | Expoxy- |
| | Paspa- | Terpen- | Terpen- | Terpen- | Terpen- | Terpen- | Terpen- | Janthi- | Janthi- | Janthi- | Janthi- | Janthi- |
| | line¹ | dole E¹ | dole I¹ | dole J¹ | dole C¹ | dole K¹ | dole N¹ | trinol | trem I | trem II | trem III | trem IV |
| AR37 WT | 0.1 | 0.0 | 0.0 | 0.0 | 0.0 | 0.0 | 0.0 | 2.9 | 40.2 | 10.2 | 13.2 | 19.1 |
| AR37 idtDg63 | 0.3 | 0.1 | 0.3 | 0.0 | 0.2 | 3.6 | 5.2 | 0.0 | 0.0 | 0.0 | 0.0 | 0.0 |
| AR37 idtDg148 | 0.2 | 0.1 | 0.3 | 0.0 | 0.3 | 3.0 | 3.7 | 0.0 | 0.0 | 0.0 | 0.0 | 0.0 |
| AR37 idtA g64 | 0.1 | 0.0 | 0.0 | 0.0 | 0.0 | 0.0 | 0.0 | 10.5 | 0.9 | 5.6 | 15.9 | 0.0 |
| AR37 idtA g101 | 0.2 | 0.1 | 0.0 | 0.0 | 0.0 | 0.0 | 0.0 | 15.8 | 0.7 | 6.8 | 24.7 | 0.0 |

¹Concentration is relative to a Paxilline external standard.

These data clearly establish that the methods exemplified here enable the production of one or more epoxy-janthitrem compounds to be modulated in a transgenic host. As shown above, certain transgenic AR37-based strains are competent to produce one or more desired epoxy-janthitrem compounds, for example epoxy-janthitrem III, or epoxy-janthitriol, without the concomitant production of one or more other epoxy-janthitrem compounds such as epoxy-janthitrem I, or epoxy-janthitrem IV.

### Example 10

This example describes the generation of modified hosts in which one or more of the genes involved in the epoxy-janthitrem biosynthetic pathway have been modified.

Materials and Methods

Bacterial strains, fungal strains, growth conditions therefor, isolation and cloning methods, and protoplasting and transformation were as described above in Example 8 and Example 9, with the following additional details.

Perennial ryegrass (Lolium perenne cv. Samson) was inoculated with Epichloë taxonomic group LpTG-3 strain AR37 wild type and independent epoxy-janthitrem idtO or idtF CRISPR-Cas9 gene edited mutants, and with an epoxy-janthitrem idtA/idtF CRISPR-Cas9 double gene edited mutant. Seedlings were again grown in proprietary potting mixture in 90 cm pots under glasshouse conditions for 6 weeks and assessed for endophyte infection by immunoblotting as set out in Example 8 above.

CRISPR-Cas9 Protospacer Design and Cloning

The AR37 epoxy-janthitrem idtO and idtF genes were screened for CRISPR-Cas9 target sites with the protospacer adjacent motif (PAM (NGG)) sequence using Geneious (Biomatters Ltd). Two protospacer sequences (with no predicted off-site targets) targetting each gene were selected. Table 15 below presents the sequences of these protospacer sequences, in which the PAM (NGG) sequence is shown in parentheses.

TABLE 15
CRISPR-Cas9 target sites (PAM (NGG)-R37 idtO
& idtF

| | | SEQ ID |
|---|---|---|
| Primer | Sequence | NO: |
| idtO g119 | AGAGACTGTGGCCACCTGAG(CGG) | 54 |
| idtO g144 | TCAACCAGTACGATGAAGCA(CGG) | 55 |
| idtF g86 | TGTAAGATAGGATGGCCCAG(TGG) | 56 |
| idtF g119 | ATTCAAGGCTTCGTACCTGA(GGG) | 57 |

Primers were designed for each protospacer of the idtO gene and the idtF gene (see Table 16 below), with the appropriate Sapl overhang for cloning into the pCas9HygAMA-ccdB vector as described in Example 9.

TABLE 16
Protospacer oligonucleotides-idtO and idtF

| | | SEQ ID |
|---|---|---|
| Primer | Sequence | NO: |
| idtO g119 Top | GTCAGAGACTGTGGCCACCTGAG | 58 |
| idtO g119 Btm | AACCTCAGGTGGCCACAGTCTCT | 59 |
| idtO g144 Top | GTCTCAACCAGTACGATGAAGCA | 60 |
| idtO g144 Btm | AACTGCTTCATCGTACTGGTTGA | 61 |
| idtF g86 Top | GTCTGTAAGATAGGATGGCCCAG | 62 |
| idtF g86 Btm | AACCTGGGCCATCCTATCTTACA | 63 |
| idtF g119 Top | GTCATTCAAGGCTTCGTACCTGA | 64 |
| idtF g119 Btm | AACTCAGGTACGAAGCCTTGAAT | 65 |

Each protospacer was generated by annealing, then cloned and transformed into E. coli, and E. coli transformants were verified and selected all as described in Example 9.

Molecular Analysis of Transformants

AR37 transformants were prepared as described above and screened for CRISPR-cas9 idtO and idtF gene editing events by sequencing a PCR product comprising the targeted CRISPR gene edit site. The PCR product was amplified using PrimeSTAR GXL polymerase (Takara) using screening primers designed to anneal to sequences up to 1 kb flanking the target PAM site. The sequences of these screening primers are shown in Table 17 below.

TABLE 17
CRISPR edit screening primers

| Primer | Sequence | SEQ ID NO: |
|---|---|---|
| idtO50056F | CGGCCCAATACAAATCAGCG | 66 |
| idtO50858R | GTGCTGTCCCGGATTGAAGA | 67 |
| idtF 203092F | CCCTTTCGCTCGCTCTCTTT | 68 |
| idtF 201958R | CAGATTGCACCCCCAAGAGT | 69 |

AR37 gene edited hosts were prepared using the CRISPR/Cas9 constructs described in this example and either the AR37 wild type strain, an AR37 host carrying an idtA g64 gene edit, or an idtA g101 gene edit. The following strains were thus produced: AR37 idtO g119; AR37 idtO g144; AR37 idtF g119 #4; AR37 idtA g64/idtF g119 #4; AR37 idtA g64/idtF g119 #9; and AR37 idtA g101/idtF g86 #8.

Results:

CRISPR Gene Edits Sequence Modifications

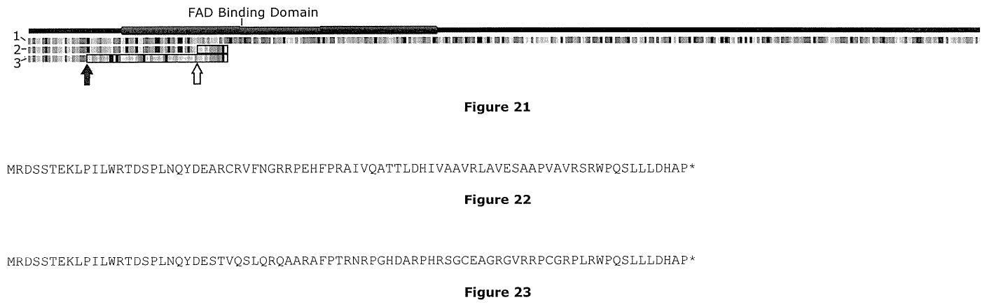

Depictions of the predicted truncated IdtO polypeptides resulting from the AR37 idtO g119 and g144 edits are presented in FIG. 21. The sites of the g119 and g144 edits are shown with a solid arrow, and an outlined arrow, respectively. Substituted amino acids resulting from the edits are boxed. The relative sizes of the truncated protein products resulting from each premature STOP codon are clearly visible.

The AR37 idtO g119 edit (the insertion of an A between A207 and G208 of SEQ ID NO: 8) leads to the substitution of amino acids followed by a premature STOP codon, as depicted in FIG. 22 and presented herein as SEQ ID NO: 70.

The AR37 idtO g144 edit (the insertion of an A between A72 and G73 of SEQ ID NO: 8) leads to the substitution of multiple different amino acids followed by a premature STOP codon, as depicted in FIG. 23 and presented herein as SEQ ID NO: 71.

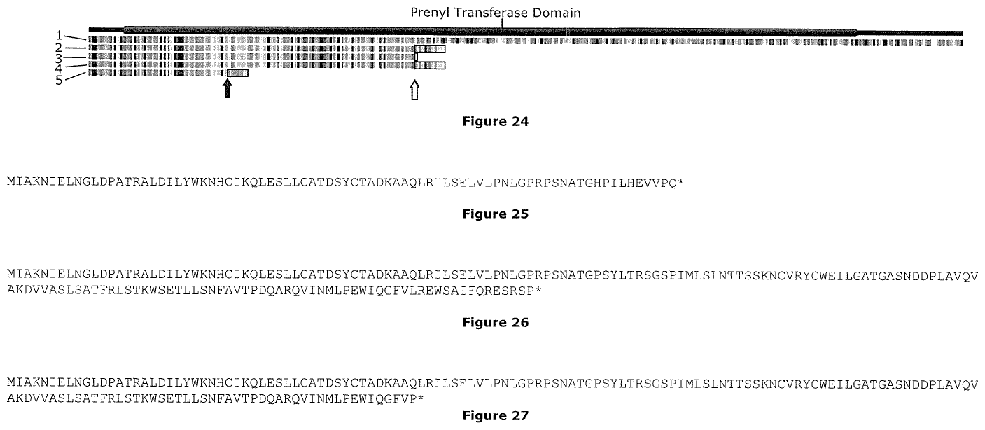

Depictions of the predicted truncated IdtF polypeptides resulting from the AR37 idtF g119 single edit, two different idtA g64/idtF g119 double gene edits (strains AR37 idtA g64/idtF g119 #4 and AR37 idtA g64/idtF g119 #9), and an idtA g64/idtF g86 double gene edit (strain AR37 idtA g101/idtF g86 #8), are presented in FIG. 24. The sites of the g86 and g119 edits are shown with a solid arrow, and an outlined arrow, respectively. Substituted amino acids resulting from the edits are boxed. The relative sizes of the truncated protein products resulting from each premature STOP codon are clearly visible.

The AR37 idtF g86 edit (the deletion of G208 of SEQ ID NO: 18) in the AR37 idtA g101/idtF g86 #8 double mutant leads to the substitution of multiple different amino acids followed by a premature STOP codon, as depicted in FIG. 25 and presented herein as SEQ ID NO: 72.

The AR37 idtF g119 gene edit resulted in the deletion of C493 of SEQ ID NO: 18 in the AR37 idtF g119 #4 single mutant strain and in the AR37 idtA g64/idtF g119 #9 double mutant strain, leading to the substitution of multiple different amino acids followed by a premature STOP codon, as depicted in FIG. 26 and presented herein as SEQ ID NO: 73. In a second double mutant, the AR37 idtA g64/idtF g119 #4 double mutant strain, this edit resulted in the insertion of a C between A492 and C493 of SEQ ID NO:18, leading to the substitution of amino acids followed by a premature STOP codon, as depicted in FIG. 27 and presented herein as SEQ ID NO: 74.

Collectively these examples clearly evidence the production of genetically modified host cells and organisms capable of producing indole diterpene compounds, including such production in a cell and organism that was not previously capable of producing such compounds, and including the production of particular epoxy-janthitrem compounds in amounts and/or combinations that differ to those produced by naturally-occuring cells and organisms that are able to produce epoxy-janthitrem compounds.

Publications
Aslanidis C, Jong P J de. Ligation-independent cloning of PCR products (LIC-PCR). Nucleic Acids Res. 1990; 18: 6069-6074. pmid:2235490
Babu, J. V., et al. Identification and Structure Elucidation of Janthitrems A and D from Penicillium janthinellum and Determination of the Tremorgenic and Anti-Insect Activity of Janthitrems A and B. Journal of Agricultural and Food Chemistry 2018, 66, 13116-13125.
Byrd, A. D., et al. The β-tubulin gene of Epichloë typhina from perennial ryegrass (Lolium perenne). Curr. Genet. 1990, 18, 347-354.
Fleetwood D. J., et al. A complex Ergovaline Gene Cluster in Epichloë Endophytes of grasses. Applied and Environmental Microbiology April 2007 p2571-2579, 2007.
Gallagher, R. T.; Hawkes, A. D. Estimation of neurotoxin levels in perennial ryegrass by mouse bioassay. New Zealand Journal of Agricultural Research 1985, 28, 427-431.
Gallagher, R. T.; Hawkes, A. D., The potent tremorgenic neurotoxins lolitrem B and aflatrem: a comparison of the tremor response in mice. Experientia 1986, 42, 823-825.
Gwinn, K. D., et al. Tissue print-immunoblot, an accurate method for the detection of Acremonium coenophialum in tall fescue. Phytopathology 81:747-748, 1991.
Kulkarni R.K. and Nielsen B. D. Nutritional requirements for growth of a fungus endophyte of tall fescue grass. Mycologia, 78(5), 1986, pp. 781-786, 1986.
Latch, G. C. M., and M. J. Christensen. Artificial infection of grasses with endophytes. Ann. Appl. Biol. 107:17-24, 1985.
Miles, C. O., et al. Synthesis and tremorgenicity of paxitriols and lolitriol: possible biosynthetic precursors of Lolitrem B. Journal of Agricultural and Food Chemistry 1992, 40, 234-238.
Popay, A. J., A laboratory method for rearing porina. New Zealand Plant Protection 2001, 54, 251.
Rahnama M., et al. Efficient targeted mutagenesis inEpichloë festucaeusing a splitmarker system. Journal of Microbiological Methods Volume 134, March 2017, Pages 62-65.
Simpson, W. R., et al. A morphological change in the fungal symbiont Neotyphodium lolii induces dwarfing in its host plant Lolium perenne. Fungal Biol. 2012, 116, 234-240.
Song L, et al. Efficient genome editing using tRNA promoter-driven CRISPR/Cas9 gRNA in Aspergillus niger. PloS ONE 13(8):e0202868, 2018.
Storms R, et al. Plasmid vectors for protein production, gene expression and molecular manipulations in Aspergillus niger. Plasmid. 2005; 53: 191-204. pmid:15848224
Young C., et al. Paxilline-negative mutants of Penicillium paxilli generated by heterologous and homologous plasmid intergration. Curr Genet 1998 33:368-377, 1998.
Young C., et al., Molecular cloning and genetic analysis of a symbiosis-expressed gene cluster for lolitrem biosynthesis from a mutualistic endophyte of perennial ryegrass. Mol Gen Genomics (2005) 274: 13-29, 2005.

### INDUSTRIAL APPLICATION

The polynucleotides, polypeptides, expression constructs, host cells, and methods of the invention have utility in many agricultural and horticultural applications, such as providing the agricultural and horticulture sectors with a useful means of controlling plant pests, and/or conferring a benefit on one or more plants.

The entire disclosures of all applications, patents and publications cited above and below, if any, are herein incorporated by reference.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

The invention may also be said broadly to consist in the parts, elements and features referred to or indicated in the specification of the application, individually or collectively, in any or all combinations of two or more of said parts, elements or features.

Aspects of the invention have been described by way of example only, and it should be appreciated that variations, modifications and additions may be made without departing from the scope of the invention, for example when present the invention as defined in the indicative claims. Furthermore, where known equivalents exist to specific features, such equivalents are incorporated as if specifically referred in this specification.

1. A host cell comprising a polypeptide selected from the group consisting of:
a. a polypeptide comprising, consisting essentially of, or consisting of an amino acid sequence corresponding to the amino acid sequence of IdtA from Epichloë var. lolii strain AR37, or corresponding to a polypeptide encoded by the idtA gene from Epichloë festucae var. lolii strain AR37;
b. a polypeptide comprising, consisting essentially of, or consisting of an amino acid sequence corresponding to the amino acid sequence of IdtD from Epichloë festucae var. lolii strain AR37, Epichloë festucae var. lolii strain AR127, Epichloë festucae var. lolii strain AR128, or Epichloë festucae var. lolii strain AR166, or corresponding to a polypeptide encoded by the idtD gene from Epichloë festucae var. lolii strain AR37, Epichloë festucae var. lolii strain AR127, Epichloë festucae var. lolii strain AR128, or Epichloë festucae var. lolii strain AR166;
c. a polypeptide comprising, consisting essentially of, or consisting of an amino acid sequence corresponding to the amino acid sequence of IdtO from Epichloë festucae var. lolii strain AR37, or corresponding to a polypeptide encoded by the idtO gene from Epichloë festucae var. lolii strain AR37;
d. a polypeptide comprising, consisting essentially of, or consisting of an amino acid sequence corresponding to the amino acid sequence of IdtF from Epichloë festucae var. lolii strain AR37 or Epichloë festucae var. lolii strain AR6, or corresponding to a polypeptide encoded by the idtF gene from Epichloë festucae var. lolii strain AR37 or Epichloë festucae var. lolii strain AR6;
e. a polypeptide comprising, consisting essentially of, or consisting of an amino acid sequence corresponding to the amino acid sequence of IdtK from Epichloë festucae var. lolii strain AR37 or Epichloë festucae var. lolii strain AR6, or corresponding to a polypeptide encoded by the idtK gene from Epichloë festucae var. lolii strain AR37 or Epichloë festucae var. lolii strain AR6;
f. a polypeptide comprising, consisting essentially of, or consisting of an amino acid sequence set forth in any one of SEQ ID NO: 3, 6, 9, 17, 19, 21, 23, 50-53, or 70-74; or
g. a polypeptide comprising, consisting essentially of, or consisting of an amino acid sequence corresponding to at least 10 contiguous amino acids from an amino acid sequence set forth in any one of SEQ ID NO: 3, 6, 9, 17, 19, 21, 23, 50-53, or 70-74;
h. a polypeptide comprising, consisting essentially of, or consisting of an amino acid sequence corresponding to amino acid residues 150 to 239 of SEQ ID NO: 3; or
i. a polypeptide comprising, consisting essentially of, or consisting of an amino acid sequence corresponding to at least 10 contiguous amino acids from an amino acid sequence set forth in amino acid residues 150 to 239 of SEQ ID NO: 3;
j. a polypeptide comprising, consisting essentially of, or consisting of an amino acid sequence corresponding to amino acid residues 76 to 436 of SEQ ID NO: 6; or
k. a polypeptide comprising, consisting essentially of, or consisting of an amino acid sequence corresponding to at least 10 contiguous amino acids from an amino acid sequence set forth in amino acid residues 76 to 436 of SEQ ID NO: 6;
l. a polypeptide comprising, consisting essentially of, or consisting of an amino acid sequence corresponding to amino acid residues 39 to 174 of SEQ ID NO: 9; or
m. a polypeptide comprising, consisting essentially of, or consisting of an amino acid sequence corresponding to at least 10 contiguous amino acids from an amino acid sequence set forth in amino acid residues 39 to 174 of SEQ ID NO: 9;
n. a polypeptide comprising, consisting essentially of, or consisting of an amino acid sequence corresponding to amino acid residues 19 to 386 of SEQ ID NO: 17 or 19; or
o. a polypeptide comprising, consisting essentially of, or consisting of an amino acid sequence corresponding to at least 10 contiguous amino acids from an amino acid sequence set forth in amino acid residues 19 to 386 of SEQ ID NO: 17 or 19;
p. a polypeptide comprising, consisting essentially of, or consisting of an amino acid sequence corresponding to amino acid residues 353 to 487 of SEQ ID NO: 21 or 23; or
q. a polypeptide comprising, consisting essentially of, or consisting of an amino acid sequence corresponding to at least 10 contiguous amino acids from an amino acid sequence set forth in amino acid residues 353 to 487 of SEQ ID NO: 21 or 23;
r. a polypeptide comprising, consisting essentially of, or consisting of an amino acid sequence encoded by a polynucleotide sequence set forth in any one of SEQ ID NO: 1, 2, 4, 5, 7, 8, 10 to 16, 18, 20, or 22;
s. a polypeptide encoded by a polynucleotide sequence having at least about 90% nucleic acid sequence identity to a sequence set forth in any one of SEQ ID NO: 1, 2, 4, 5, 7, 8, 10 to 16, 18, 20, or 22;
t. a polypeptide involved in the production of one or more janthitrem compound, including a janthitrem compound of formulae VI, or an epoxy-janthitrem compound of any one of formulae Ito III or V to VIII;
u. a polypeptide comprising an enzymatic activity having as its substrate terpendole I or as its substrate or its product a compound of any one of formulae Ito VIII, such as a compound of any one of formulae IIA to IIE;
v. a catalytically active fragment of any one of a) to u) above; and
w. a polypeptide comprising, consisting essentially of, or consisting of a sequence of amino acid residues that has at least 70%, at least 75%, at least 80%, at least 85%, or at least 90% or greater amino acid sequence identity to any one of a) to t) above; or
x. any combination of two or more of a) to w) above;
wherein the polypeptide is heterologous to the cell or is heterologously expressed by the cell.

2. An isolated, purified, recombinant, or synthesised polypeptide as defined in claim 1.

3. An isolated, purified, recombinant, or synthesised polynucleotide encoding a polypeptide as defined in claim 1.

4. An isolated, purified, recombinant, or synthesised polynucleotide comprising at least about 90% nucleic acid sequence identity to the nucleic acid sequence set forth in any one of SEQ ID NO: 1, 2, 4, 5, 7, 8, 10 to 16, 18, 20, or 22.

5. A polynucleotide comprising a nucleic acid sequence encoding a polypeptide and a transcription control sequence, wherein the polypeptide comprises an amino acid sequence at least 90% identical to any one of SEQ ID NO: 3, 6, 9, 17, 19, 21, 23, 50-53, or 70-74, and/or wherein the polypeptide catalyses the conversion of a substrate in the epoxy-janthitrem biosynthetic pathway, and wherein the nucleic acid sequence encoding the polypeptide is heterologous to the transcription control sequence.

6. A vector capable of expressing a polypeptide as defined in claim 1, or comprising a polynucleotide encoding a polypeptide as defined in claim 1.

7. A host cell comprising a polynucleotide according to any one of claims 3 to 5 or a vector according to claim 6, wherein the polynucleotide is heterologous to the host cell.

8. The host cell of claim 1 or claim 7, wherein the heterologous polypeptide is encoded by a nucleic acid sequence heterologous to the host cell.

9. The host cell of any one of claim 1, 7 or 8, wherein the polypeptide catalyses the production of an epoxy-janthitrem compound of formula I or formula II.

10. The host cell according to any preceding claim, wherein the host cell comprises an endophytic symbiont.

11. The host cell according to any preceding claim, wherein the host cell is an Epichloë cell.

12. The host cell according to claim 11, wherein in the absence of the polypeptide, the Epichloë cell is unable to synthesise one or more epoxy-janthitrem compounds and/or is unable to synthesise a compound of formula I or formula II.

13. The host cell of any one of claims 1 or 7 to 12, wherein the host cell comprises one or more functional genes selected from the group comprising idtG, idtM, idtB, idtC, idtP, idtQ, idtF, and idtK.

14. The host cell of claim 13, wherein the host cell comprises each of the genes from the group comprising idtG, idtM, idtB, idtC, idtP, and idtQ.

15. The host cell of any one of claims 1 or 7 to 14, wherein the host cell comprises the gene idtF.

16. The host cell of any one of claims 1 or 7 to 15, wherein the host cell comprises the gene idtK.

17. A method for preparing a polypeptide that catalyses the conversion of a substrate in the epoxy-janthitrem biosynthetic pathway, the method comprising the step of culturing a host cell of any preceding claim under conditions that provide for expression of the polypeptide.

18. The method of claim 17, further comprising purifying the polypeptide.

19. A method of making an epoxy-janthitrem compound, comprising:
a. contacting an indole diterpene precursor with a polypeptide as defined in claim 1 to produce an epoxy-janthitrem compound; and
b. optionally, isolating the epoxy-janthitrem compound produced in step (a);
wherein if the epoxy-janthitrem compound is produced in a host cell, the polypeptide is heterologous to the host cell or is heterologously expressed by the host cell.

20. The method of claim 19, wherein when produced in a host cell, the polypeptide as defined in claim 1 is heterologous to the cell and the indole diterpene precursor is present in and/or expressed by the same cell as the polypeptide, and the step of contacting the indole diterpene precursor occurs in the host cell.

21. The method of claim 19 or 20, further comprising isolating an epoxy-janthitrem compound produced by the host cell.

22. The method of any one of claims 19 to 21, wherein the indole diterpene precursor is selected from the group comprising isopentyl pyrophosphate, farnesyl pyrophosphate, and indole-3-glycerol phosphate.

23. The method of any one of claims 19 to 22, wherein the indole diterpene precursor is selected from the group comprising terpendole C, terpendole J, terpendole I, terpendole B, terpendole G, terpendole F, terpendole E, α-paxitriol, α-PC-M6, paspaline B, 12′-hydroxy-paspaline, paspaline, and emindole SB, or any combination thereof.

24. A method of providing or modifying production of one or more indole diterpene compounds in a host cell or organism, wherein the host cell or organism comprises at least one genetic modification associated with altered regulation or production of one or more gene products encoded by a gene selected from the group consisting of idtA, idtD, idtF, and idtO.

25. The method as claimed in claim 24 wherein the method comprises introducing into said host cell or organism at least one genetic modification in an idtA gene that reduces or prevents the production or activity of an idtA gene product, wherein the production or amount of one or more epoxy janthitrem compounds is decreased when compared a host cell or organism in which such a genetic modification in an idtA gene is not present.

26. The method as claimed in claim 24 or claim 25 wherein the method comprises introducing into said host cell or organism at least one genetic modification in an idtD gene that reduces or prevents the production or activity of an idtD gene product, wherein the production or amount of one or more epoxy janthitrem compounds is decreased when compared a host cell or organism in which such a genetic modification in an idtD gene is not present.

27. A method of providing or increasing production of one or more epoxy-janthitrem compounds in a host cell or organism comprising a functional cytochrome P450 monooxygenase activity, such as a functional cytochrome P450 monooxygenase encoded by the idtQ gene, the method comprising: expressing a polypeptide as defined in claim 1 in the host cell or organism under conditions effective to produce one or more epoxy-janthitrem compounds, wherein said polypeptide is heterologous to the host cell or organism, and wherein the polypeptide as defined in claim 1 replaces an inactive or deleted activity, introduces a new activity, or enhances an existing activity in the host cell or organism, and wherein production of one or more epoxy-janthitrem compounds in the host cell or organism is provided or increased.

28. A method of providing or increasing production of one or more epoxy-janthitrem compounds in a host cell or organism capable of terpendole I production comprising: expressing a polypeptide as defined in claim 1 in the host cell or organism under conditions effective to produce one or more epoxy-janthitrem compounds, wherein said polypeptide is heterologous to the host cell or organism, and wherein the polypeptide as defined in claim 1 replaces an inactive or deleted activity, introduces a new activity, or enhances an existing activity in the host cell or organism, and wherein production of one or more epoxy-janthitrem compounds in the host cell or organism is provided or increased.

29. The method of any one of claims 20 to claim 28, wherein the host cell or organism comprises one or more functional genes selected from the group comprising idtG, idtM, idtB, idtC, idtP, idtQ, idtF, and idtK.

30. The method of any one of claims 20 to claim 29, wherein the host cell or organism comprises each of the genes from the group comprising idtG, idtM, idtB, idtC, idtP, and idtQ.

31. The method of any one of claims 20 to claim 30, wherein the host cell or organism comprises the gene idtF.

32. The method of any one of claims 20 to 31, wherein the host cell or organism comprises the gene idtK.

33. The method of any one of claims 20 to 32, wherein the host cell or organism comprises a polypeptide comprising an amino acid sequence having at least 90% identity with an amino acid sequence set forth in any one of SEQ ID NO: 3, 6, 9, 17, 19, 21, 23, 50-53, or 70-74, wherein the polypeptide is heterologous to the host cell,

34. The method of any one of claims 20 to 33, wherein the host cell or organism comprises a heterologous polypeptide which catalyzes one of the following reactions:
a. the conversion of terpendole I to epoxy-janthitriol;
b. the conversion of terpendole J to epoxy-janthitrem III;
c. the conversion of terpendole C to epoxy-janthitrem II;
d. the conversion of epoxy-janthitriol to epoxy-janthitrem I;
e. the conversion of epoxy-janthitrem III to epoxy-janthitrem IV; or
f. any combination of two or more of a) to e) above.

35. The method of any one of claims 19 to 34, wherein the epoxy-janthitrem compound is a compound of formula I or formula II.

36. The method of any one of claims 19 to 35, wherein the epoxy-janthitrem compound is selected from the group consisting of epoxy-janthitrem I, epoxy-janthitrem II, epoxy-janthitrem III, and epoxy-janthitrem IV.

37. The method of any one of claims 19 to 36, wherein a mixture of epoxy-janthitrem compounds is produced.

38. The method of any one of claims 19 to 37, wherein one or more of epoxy-janthitriol, epoxy-janthitrem II, epoxy-janthitrem III, and epoxy-janthitrem IV are produced.

39. The method of claim 38 wherein epoxy-janthitrem II, epoxy-janthitrem III, and epoxy-janthitrem IV are produced.

40. The method of any one of claims 19 to 39, wherein
a. epoxy-janthitriol is not substantially produced; or
b. epoxy-janthitrem I is not substantially produced; or
c. epoxy-janthitrem II is not substantially produced; or
d. epoxy-janthitrem III is not substantially produced; or
e. epoxy-janthitrem IV is not substantially produced; or
f. any combination of any two or more of a) to e) above.

41. The method of claim 40, wherein epoxy-janthitrem I is not substantially produced.

42. A genetically modified host cell capable of producing one or more indole diterpene compounds, wherein the host cell comprises at least one genetic modification associated with altered regulation or production of one or more gene products encoded by a gene selected from the group consisting of idtA, idtD, idtO, idtG, idtM, idtB, idtC, idtP, idtQ, idtF, and idtK.

43. The host cell or organism as claimed in claim 40 comprising at least one genetic modification associated with altered regulation or production of one or more gene products encoded by a gene selected from the group consisting of idtA, idtD, and idtO.

44. A genetically modified host cell comprising a polypeptide comprising an amino acid sequence having at least 90% identity with an amino acid sequence set forth in any one of SEQ ID NO: 3, 6, 9, 17, 19, 21, 23, 50-53, or 70-74, wherein the polypeptide is heterologous to the host cell, and wherein the polypeptide catalyzes one of the following reactions:
g. the conversion of terpendole I to epoxy-janthitriol;
h. the conversion of terpendole J to epoxy-janthitrem III;
i. the conversion of terpendole C to epoxy-janthitrem II;
j. the conversion of epoxy-janthitriol to epoxy-janthitrem I;
k. the conversion of epoxy-janthitrem III to epoxy-janthitrem IV; or
l. any combination of two or more of a) to e) above.

45. The genetically modified host cell of claim 42, wherein the host cell is capable of synthesizing a compound of any one of formulae IV to VIII or uptaking a compound of any one of formulae IV to VIII from its surroundings.

46. The genetically modified host cell of claim 42 or claim 45, wherein the host cell further comprises one or more enzymes of a pathway for synthesizing a compound of any one of formulae IV to VIII from a carbon source.

47. The genetically modified host cell of claim 46, wherein the pathway for synthesizing a compound of formula II from a carbon source is native to the host cell.

48. The genetically modified host cell of claim 46, wherein the pathway for synthesizing a compound of formula II from a carbon source is heterologous to the host cell.

49. The genetically modified host cell of claim 42, wherein the polypeptide comprises, consists essentially of, or consists of an amino acid sequence having at least 75% identity with an amino acid sequence set forth in any one of SEQ ID NO: 3, 6, 9, 17, 19, 21, 23, 50-53, or 70-74.

50. The genetically modified host cell of claim 49, wherein the polypeptide comprises, consists essentially of, or consists of an amino acid sequence having at least 95% identity with the amino acid sequence set forth in any one of SEQ ID NO: 3, 6, 9, 17, 19, 21, 23, 50-53, or 70-74.

51. The genetically modified host cell of any one of claims 42 to 50, wherein the host cell is an Epichloë cell.

52. A method of producing a compound of formula I in a genetically modified host cell, comprising:
a. providing the genetically modified host cell of any one of claims 42 to 50; and
b. culturing the genetically modified host cell in a medium under a suitable condition; or
c. maintaining the genetically modified host cell of any one of claims 42 to 50 under a suitable condition;
wherein the culturing or the maintaining results in the genetically modified host cell producing a compound of formula I or formula II.

53. The method of claim 52, further comprising separating a compound of formula I from the host cell and/or the medium, wherein the separating step is subsequent, concurrent or partially concurrent with the culturing or maintaining step.

54. The method of claim 52, wherein the maintaining is in the presence of one or more cells other than the genetically modified host cell.

55. The method of claim 54, wherein the genetically modified host cell is maintained together with one or more plant or animal cells.

56. An Epichloë cell, wherein the Epichloë cell:
a. has been modified or transformed with one or more polynucleotides encoding a polypeptide as defined in claim 1; or
b. is capable of heterologously expressing one or more polypeptides as defined in claim 1; or
c. comprises a polynucleotide encoding a polypeptide as defined in claim 1; or
d. comprises a polynucleotide comprising at least about 75%, for example at least about 90% nucleic acid sequence identity to the nucleic acid sequence set forth in any one of SEQ ID NO: 1, 2, 4, 5, 7, 8, 10 to 16, 18, 20, or 22; or
e. is genetically modified to confer upon the Epichloë cell the capacity to synthesize one or more epoxy-janthitrem compounds, wherein the Epichloë cell did not have such capacity in the absence of the genetic modification; or
f. comprises at least one genetic modification associated with altered regulation or production of one or more gene products encoded by a gene selected from the group consisting of idtA, idtD, idtO, idtG, idtM, idtB, idtC, idtP, idtQ, idtF, and idtK; or
g. comprises at least one genetic modification associated with altered regulation or production of one or more gene products encoded by a gene selected from the group consisting of idtA, idtD, and idtO; or
h. comprises a genetic modification introduced by gene editing using any one or more of the oligonucleotides, polynucleotides, constructs, or vectors as described herein in the Examples or as presented herein in any one of SEQ ID NOs: 24 to 49; or
i. any combination of two of more of a) to h) above.

57. The Epichloë cell of claim 56, wherein the one or more epoxy-janthitrem compounds is a compound of formula I or formula II.

58. A population of cells comprising one or more plant cells and one or more Epichloë cells as claimed in claim 56 or claim 57.

59. The population of cells as claimed in claim 58, wherein the one or more plant cells comprise, consist essentially of, or consist of one or more cells from the group consisting of:
a. a Pooideae grass;
b. a perennial ryegrass;
c. an annual ryegrass;
d. a hybrid ryegrass;
e. the genus Lolium;
f. the species Lolium perenne, Lolium multiflorum, and Lolium x hybridum;
g. the genus Festuca;
h. the species Festuca amethystina, Festuca arundinacea, Festuca cinerea, Festuca elegans, Festuca glauca, Festuca idahoensis, Festuca ovine, Festuca pallens, Festuca pratensis, Festuca rubra, Festuca rubra subsp. commutate, Festuca saximontana, and Festuca trachyphylla.

60. The population of cells as claimed in claim 58 or claim 59, said population comprising a plant or part thereof.

61. A method for conferring on an organism the ability to produce one or more epoxy-janthitrem compounds, the method comprising providing the organism with a host cell modified or transformed with or to comprise one or more polynucleotides encoding a polypeptide as defined in claim 1, or one or more genes involved in the epoxy-janthitrem biosynthetic pathway.
